(19) United States
(12) Patent Application Publication
(10) Pub. No.: US 2026/0282744 A1
(43) Pub. Date: Sep. 17, 2026

(54) ORGANIC ELECTROLUMINESCENT ELEMENT AND ELECTRONIC DEVICE

(71) Applicant: IDEMITSU KOSAN CO.,LTD., Tokyo (JP)

(72) Inventors: Ryota TAKAHASHI, Chiyoda-ku, Tokyo (JP); Tomonori NAKAMURA, Chiyoda-ku, Tokyo (JP); Sayaka MIZUTANI, Chiyoda-ku, Tokyo (JP); Masato MITANI, Chiyoda-ku, Tokyo (JP)

(73) Assignee: IDEMITSU KOSAN CO.,LTD., Tokyo (JP)

(21) Appl. No.: 19/163,206

(22) PCT Filed: Mar. 13, 2024
(86) PCT No.: PCT/JP2024/009774

§ 371 Date: Sep. 8, 2025

(30) Foreign Application Priority Data
Mar. 14, 2023 (JP) 2023-039701
Mar. 14, 2023 (JP) 2023-039757

(51) Int. Cl. H10K 85/60; H10K 50/16

(52) U.S. Cl. CPC H10K 85/6574; H10K 85/615; H10K 50/166

(57) ABSTRACT

Provided is an organic electroluminescent element having a cathode, an anode, and one or more organic layers disposed between the cathode and the anode, wherein: at least one layer among the one or more organic layers includes a first component and a second component; the first component is the compound represented by formula (1); and the second component is selected from the group consisting of alkali metals, alkali metal compounds, alkaline earth metals, alkaline earth metal compounds, rare earth metals, rare earth metal compounds, organometallic complexes including an alkali metal, organometallic complexes including an alkaline earth metal, and organometallic complexes including a rare earth metal.

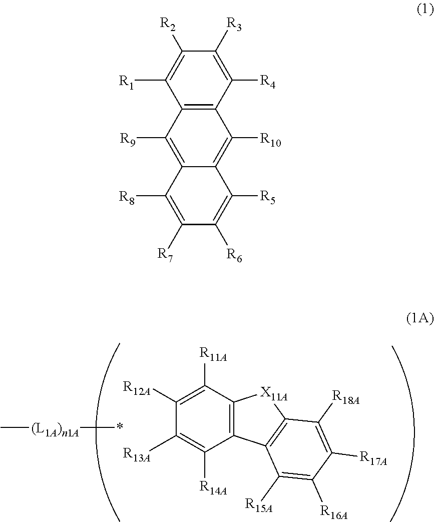

### TECHNICAL FIELD

The present invention relates to an organic electroluminescence device and an electronic apparatus.

### BACKGROUND ART

When a voltage is applied to an organic electroluminescence device (hereinafter, also referred to as “organic EL device”), holes are injected from an anode and electrons are injected from a cathode into an emitting layer. The injected holes and electrons are recombined in the emitting layer to form excitons.

Conventional organic EL devices have not yet achieved sufficient device performance. In order to enhance the device performance, improvement of the organic EL devices has been gradually advanced, but further improvement in performance is required.

Patent Document 1 discloses that a compound having a specific structure is used for an electron-transporting layer of an organic EL device.

### RELATED ART DOCUMENTS

### Patent Documents

[Patent Document 1] CN 114122299 A

### SUMMARY OF INVENTION

An object of the present invention is to provide an organic EL device with higher performance.

As a result of intensive studies to achieve the above object, the present inventors have found that an organic EL device that has a low driving voltage and high efficiency from a low current density to a high current density can be obtained by using two specific components in combination in at least one layer of organic layers of the organic EL device, and have completed the present invention.

In addition, as a result of intensive studies to achieve the above object, the present inventors have found that an organic EL device having a low driving voltage and high efficiency at a low current density can be obtained by using two specific components in combination in at least one layer of organic layers of the organic EL device, and have completed the present invention.

According to the present invention, the following organic EL device and the like are provided.

1. An organic electroluminescence device comprising
a cathode,
an anode, and
one or two or more organic layers disposed between the cathode and the anode,
wherein at least one layer of the one or two or more organic layers comprises a first component and a second component,
the first component is a compound represented by the following formula (1), and
the second component is selected from the group consisting of an alkali metal, an alkali metal compound, an alkaline earth metal, an alkaline earth metal compound, a rare earth metal, a rare earth metal compound, an organic metal complex containing an alkali metal, an organic metal complex containing an alkaline earth metal, and an organic metal complex containing a rare earth metal:

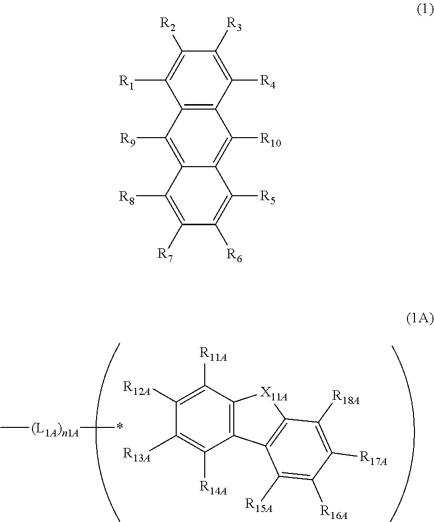

wherein in the formula (1),
at least one of R₁ to R₁₀ is a group represented by the formula (1A);
R₁ to R₁₀ that are not the group represented by the formula (1A) are independently a hydrogen atom, or a substituent A; provided that at least three of R₁ to R₁₀ are independently the group represented by the formula (1A), the substituent A, or a hydrogen atom which is a deuterium atom;
in the formula (1A),
L_{1A} is
a single bond,
a substituted or unsubstituted arylene group having 6 to 50 ring carbon atoms, or
a substituted or unsubstituted divalent heterocyclic group having 5 to 50 ring atoms;
n1A is an integer of 0 to 3;
when n1A is 0, (L_{1A})_{n1A} is a single bond;
when n1A is 2 or 3, the plurality of L_{1A}'s is connected in series with each other, and a structure in parentheses is bonded to L_{1A} farthest from the anthracene skeleton; the plurality of L_{1A}'s may be the same as or different from each other;
X_{11A} is C(R_{21A})(R_{22A}), N(R_{23A}), O, or S;
one or more sets of the adjacent two or more of R_{11A} to R_{18A} form a substituted or unsubstituted single ring by bonding with each other, form a substituted or unsubstituted fused ring by bonding with each other, or do not form any of the rings;
when the substituted or unsubstituted single ring is formed, one of atoms forming the single ring is bonded to L_{1A}, or one of R_{11A} to R_{18A} that do not contribute to the formation of the single ring, and R_{21A} to R_{23A} represents a bond to L_{1A};
when the substituted or unsubstituted fused ring is formed, one of atoms forming the fused ring is bonded to L_{1A}, or one of R_{11A} to R_{18A} that do not contribute to the formation of the fused ring, and R_{21A} to R_{23A} represents a bond to L_{1A};
when the single ring and the fused ring are not formed, one of R_{11A} to R_{18A} and R_{21A} to R_{23A} represents a bond to L_{1A}; R_{11A} to R_{18A} and R_{21A} to R_{23A} that do not represent the bond to L_{1A} and that do not form any of the rings are independently a hydrogen atom or a substituent A;
when two or more groups represented by the formula (1A) are present, the two or more groups represented by the formula (1A) may be the same as or different from each other;
the substituent A is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,

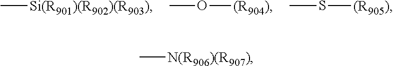

a halogen atom, a nitro group,
a substituted or unsubstituted aryl group having 6 to 50 ring carbon atoms, or
a substituted or unsubstituted monovalent heterocyclic group having 5 to 50 ring atoms; R₉₀₁ to R₉₀₇ are independently a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more substituents A are present, the two or more substituents A may be the same as or different from each other; and
when two or more of each of R₉₀₁ to R₉₀₇ are present, the two or more of each of R₉₀₁ to R₉₀₇ may be the same as or different from each other,
provided that the compound represented by the formula (1) does not have a structure represented by the following formula (M1), a structure represented by the following formula (M2), a structure represented by the following formula (M3), and a structure represented by the following formula (M4) in the molecule thereof:

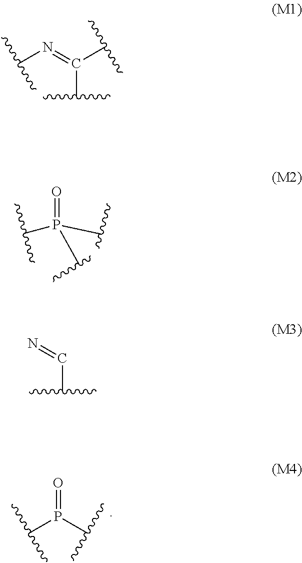

2. An organic electroluminescence device comprising
a cathode,
an anode, and
one or two or more organic layers disposed between the cathode and the anode,
wherein at least one layer of the one or two or more organic layers includes a first component and a second component,
the first component is a compound that satisfies the following formulas (R1) and (R2) and does not have structures represented by the following formulas (M1) to (M4) in the molecule thereof, and
the second component is selected from the group consisting of an alkali metal, an alkali metal compound, an alkaline earth metal, an alkaline earth metal compound, a rare earth metal, a rare earth metal compound, an organic metal complex containing an alkali metal, an organic metal complex containing an alkaline earth metal, and an organic metal complex containing a rare earth metal:

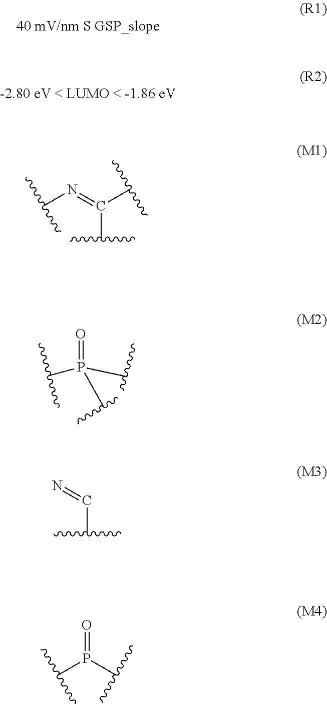

wherein in the formula (R1), GSP_slope represents a giant surface polarization slope; and in the formula (R2), LUMO represents a lowest unoccupied molecular orbital energy level.

3. An electronic apparatus comprising the organic electroluminescence device according to the above 1 or 2.

4. A compound represented by the following formula (2):

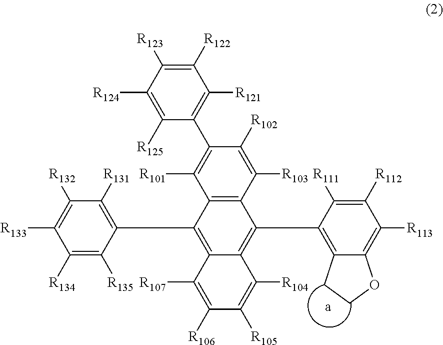

wherein in the formula (2),
a ring a is
a substituted or unsubstituted aromatic hydrocarbon ring having 10 to 50 ring carbon atoms, or
a substituted or unsubstituted heterocyclic ring having 6 to 50 ring atoms; R₁₀₁ to R₁₀₇, R₁₁₁ to R₁₁₃, R₁₂₁ to R₁₂₅, and R₁₃₁ to R₁₃₅ are independently a hydrogen atom, or a substituent R,
provided that at least one of R₁₁₁ to R₁₁₃ is the substituent R, or the ring a has at least one substituent;
the substituent R is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,

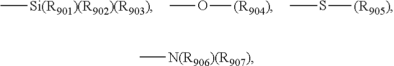

a halogen atom, a cyano group, a nitro group,
a substituted or unsubstituted aryl group having 6 to 50 ring carbon atoms, or
a substituted or unsubstituted monovalent heterocyclic group having 5 to 50 ring atoms;
R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more of each of R₉₀₁ to R₉₀₇ are present, the two or more of each of R₉₀₁ to R₉₀₇ may be the same as or different from each other; and
when two or more substituents R are present, the two or more substituents R may be the same as or different from each other.

According to the present invention, the organic EL device with higher performance can be provided.

### BRIEF DESCRIPTION OF DRAWINGS

FIG. 1 is a view illustrating a schematic configuration of an organic EL device according to an aspect of the present invention.

### DESCRIPTION OF EMBODIMENTS

### Definition

In this specification, a hydrogen atom includes its isotopes different in the number of neutrons, namely, a protium, a deuterium and a tritium.

In this specification, at a bondable position in a chemical formula where a symbol such as “R”, or “D” representing a deuterium atom is not indicated, a hydrogen atom, that is, a protium atom, a deuterium atom or a tritium atom is bonded.

In this specification, the number of ring carbon atoms represents the number of carbon atoms forming a subject ring itself among the carbon atoms of a compound having a structure in which atoms are bonded in a ring form (for example, a monocyclic compound, a fused ring compound, a cross-linked compound, a carbocyclic compound, or a heterocyclic compound). When the subject ring is substituted by a substituent, the carbon contained in the substituent is not included in the number of ring carbon atoms. The same shall apply to “the number of ring carbon atoms” described below, unless otherwise specified. For example, a benzene ring has 6 ring carbon atoms, a naphthalene ring includes 10 ring carbon atoms, a pyridine ring includes 5 ring carbon atoms, and a furan ring includes 4 ring carbon atoms. Further, for example, a 9,9-diphenylfluorenyl group includes 13 ring carbon atoms, and a 9,9′-spirobifluorenyl group includes 25 ring carbon atoms.

When a benzene ring is substituted by, for example, an alkyl group as a substituent, the number of carbon atoms of the alkyl group is not included in the number of ring carbon atoms of the benzene ring. Therefore, the number of ring carbon atoms of the benzene ring substituted by the alkyl group is 6. When a naphthalene ring is substituted by, for example, an alkyl group as a substituent, the number of carbon atoms of the alkyl group is not included in the number of ring carbon atoms of the naphthalene ring. Therefore, the number of ring carbon atoms of the naphthalene ring substituted by the alkyl group is 10.

In this specification, the number of ring atoms represents the number of atoms forming a subject ring itself among the atoms of a compound having a structure in which atoms are bonded in a ring form (for example, the structure includes a monocyclic ring, a fused ring and a ring assembly) (for example, a monocyclic compound, a fused ring compound, a cross-linked compound, a carbocyclic compound and a heterocyclic compound). The number of ring atoms does not include atoms which do not form the ring (for example, a hydrogen atom which terminates a bond of the atoms forming the ring), or atoms contained in a substituent when the ring is substituted by the substituent. The same shall apply to “the number of ring atoms” described below, unless otherwise specified. For example, the number of atoms of a pyridine ring is 6, the number of atoms of a quinazoline ring is 10, and the number of a furan ring is 5. For example, hydrogen atoms bonded to a pyridine ring and atoms constituting a substituent substituted on the pyridine ring are not included in the number of ring atoms of the pyridine ring. Therefore, the number of ring atoms of a pyridine ring with which a hydrogen atom or a substituent is bonded is 6. For example, hydrogen atoms and atoms constituting a substituent which are bonded with a quinazoline ring is not included in the number of ring atoms of the quinazoline ring. Therefore, the number of ring atoms of a quinazoline ring with which a hydrogen atom or a substituent is bonded is 10.

In this specification, “XX to YY carbon atoms” in the expression “a substituted or unsubstituted ZZ group including XX to YY carbon atoms” represents the number of carbon atoms in the case where the ZZ group is unsubstituted by a substituent, and does not include the number of carbon atoms of a substituent in the case where the ZZ group is substituted by the substituent. Here, “YY” is larger than “XX”, and “XX means an integer of 1 or more and “YY” means an integer of 2 or more.

In this specification, “XX to YY atoms” in the expression “a substituted or unsubstituted ZZ group including XX to YY atoms” represents the number of atoms in the case where the ZZ group is unsubstituted by a substituent, and does not include the number of atoms of a substituent in the case where the ZZ group is substituted by the substituent. Here, “YY” is larger than XX”, and “XX” means an integer of 1 or more and “YY” means an integer of 2 or more.

In this specification, the unsubstituted ZZ group represents the case where the “substituted or unsubstituted ZZ group” is a “ZZ group unsubstituted by a substituent”, and the substituted ZZ group represents the case where the “substituted or unsubstituted ZZ group“is a” ZZ group substituted by a substituent”.

In this specification, a term “unsubstituted” in the case of “a substituted or unsubstituted ZZ group” means that hydrogen atoms in the ZZ group are not substituted by a substituent. Hydrogen atoms in a term “unsubstituted ZZ group” are a protium atom, a deuterium atom, or a tritium atom.

In this specification, a term “substituted” in the case of “a substituted or unsubstituted ZZ group” means that one or more hydrogen atoms in the ZZ group are substituted by a substituent. Similarly, a term “substituted” in the case of “a BB group substituted by an AA group” means that one or more hydrogen atoms in the BB group are substituted by the AA group.

“Substituent as Described in this Specification”

Hereinafter, the substituent described in this specification will be explained.

The number of ring carbon atoms of the “unsubstituted aryl group” described in this specification is 6 to 50, preferably 6 to 30, and more preferably 6 to 18, unless otherwise specified.

The number of ring atoms of the “unsubstituted heterocyclic group” described in this specification is 5 to 50, preferably 5 to 30, and more preferably 5 to 18, unless otherwise specified.

The number of carbon atoms of the “unsubstituted alkyl group” described in this specification is 1 to 50, preferably 1 to 20, and more preferably 1 to 6, unless otherwise specified.

The number of carbon atoms of the “unsubstituted alkenyl group” described in this specification is 2 to 50, preferably 2 to 20, and more preferably 2 to 6, unless otherwise specified.

The number of carbon atoms of the “unsubstituted alkynyl group” described in this specification is 2 to 50, preferably 2 to 20, and more preferably 2 to 6, unless otherwise specified.

The number of ring carbon atoms of the “unsubstituted cycloalkyl group” described in this specification is 3 to 50, preferably 3 to 20, and more preferably 3 to 6, unless otherwise specified.

The number of ring carbon atoms of the “unsubstituted arylene group” described in this specification is 6 to 50, preferably 6 to 30, and more preferably 6 to 18, unless otherwise specified.

The number of ring atoms of the “unsubstituted divalent heterocyclic group” described in this specification is 5 to 50, preferably 5 to 30, and more preferably 5 to 18, unless otherwise specified.

The number of carbon atoms of the “unsubstituted alkylene group” described in this specification is 1 to 50, preferably 1 to 20, and more preferably 1 to 6, unless otherwise specified.

### “Substituted or Unsubstituted Aryl Group”

Specific examples of the “substituted or unsubstituted aryl group” described in this specification (specific example group G1) include the following unsubstituted aryl groups (specific example group G1A), substituted aryl groups (specific example group G1B), and the like. (Here, the unsubstituted aryl group refers to the case where the “substituted or unsubstituted aryl group” is an “aryl group unsubstituted by a substituent”, and the substituted aryl group refers to the case where the “substituted or unsubstituted aryl group” is an “aryl group substituted by a substituent”). In this specification, in the case where simply referred as an “aryl group”, it includes both a “unsubstituted aryl group” and a “substituted aryl group.”

The “substituted aryl group” means a group in which one or more hydrogen atoms of the “unsubstituted aryl group” are substituted by a substituent. Specific examples of the “substituted aryl group” include, for example, groups in which one or more hydrogen atoms of the “unsubstituted aryl group” of the following specific example group G1A are substituted by a substituent, the substituted aryl groups of the following specific example group G1B, and the like. It should be noted that the examples of the “unsubstituted aryl group” and the examples of the “substituted aryl group” enumerated in this specification are mere examples, and the “substituted aryl group” described in this specification also includes a group in which a hydrogen atom bonded with a carbon atom of the aryl group itself in the “substituted aryl group” of the following specific group G1B is further substituted by a substituent, and a group in which a hydrogen atom of a substituent in the “substituted aryl group” of the following specific group G1B is further substituted by a substituent.

### Unsubstituted Aryl Group (Specific Example Group G1A):

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenalenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
a monovalent aryl group derived by removing one hydrogen atom from the ring structures
represented by each of the following general formulas (TEMP-1) to (TEMP-15).

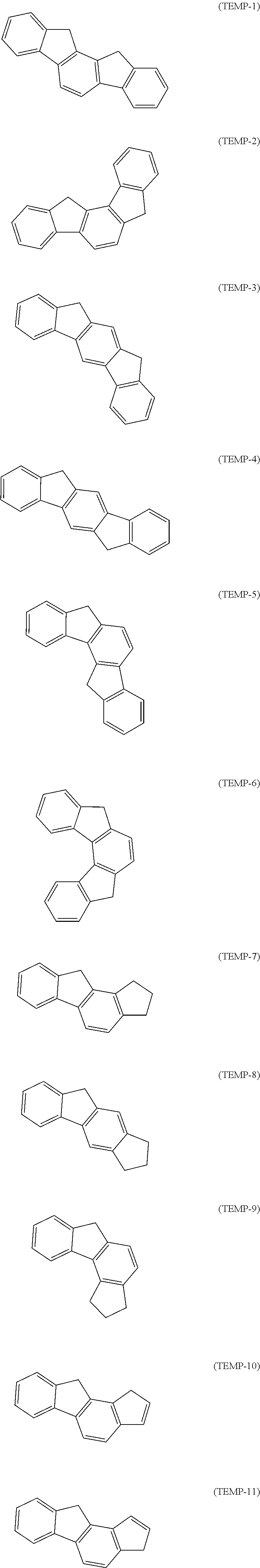

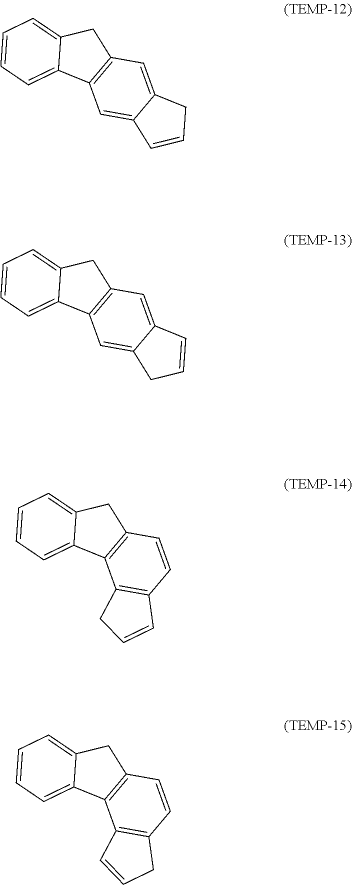

### Substituted Aryl Group (Specific Example Group G1B):

an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
an o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
a group in which one or more hydrogen atoms of a monovalent group derived from the ring structures represented by each of the general formulas (TEMP-1) to (TEMP-15) are substituted by a substituent.

### “Substituted or Unsubstituted Heterocyclic Group”

The “heterocyclic group” described in this specification is a ring group having at least one hetero atom in the ring atom. Specific examples of the hetero atom include a nitrogen atom, an oxygen atom, a sulfur atom, a silicon atom, a phosphorus atom, and a boron atom.

The “heterocyclic group” in this specification is a monocyclic group or a fused ring group.

The “heterocyclic group” in this specification is an aromatic heterocyclic group or a non-aromatic heterocyclic group.

Specific examples of the “substituted or unsubstituted heterocyclic group” (specific example group G2) described in this specification include the following unsubstituted heterocyclic group (specific example group G2A), the following substituted heterocyclic group (specific example group G2B), and the like. (Here, the unsubstituted heterocyclic group refers to the case where the “substituted or unsubstituted heterocyclic group“is a” heterocyclic group unsubstituted by a substituent”, and the substituted heterocyclic group refers to the case where the “substituted or unsubstituted heterocyclic group“is a” heterocyclic group substituted by a substituent”). In this specification, in the case where simply referred as a “heterocyclic group”, it includes both the “unsubstituted heterocyclic group” and the “substituted heterocyclic group.”

The “substituted heterocyclic group” means a group in which one or more hydrogen atom of the “unsubstituted heterocyclic group” are substituted by a substituent. Specific examples of the “substituted heterocyclic group” include a group in which a hydrogen atom of “unsubstituted heterocyclic group” of the following specific example group G2A is substituted by a substituent, the substituted heterocyclic groups of the following specific example group G2B, and the like. It should be noted that the examples of the “unsubstituted heterocyclic group” and the examples of the “substituted heterocyclic group” enumerated in this specification are mere examples, and the “substituted heterocyclic group” described in this specification includes groups in which hydrogen atom bonded with a ring atom of the heterocyclic group itself in the “substituted heterocyclic group” of the specific example group G2B is further substituted by a substituent, and a group in which hydrogen atom of a substituent in the “substituted heterocyclic group” of the specific example group G2B is further substituted by a substituent.

Specific example group G2A includes, for example, the following unsubstituted heterocyclic group containing a nitrogen atom (specific example group G2A1), the following unsubstituted heterocyclic group containing an oxygen atom (specific example group G2A2), the following unsubstituted heterocyclic group containing a sulfur atom (specific example group G2A3), and the monovalent heterocyclic group derived by removing one hydrogen atom from the ring structures represented by each of the following general formulas (TEMP-16) to (TEMP-33) (specific example group G2A4).

Specific example group G2B includes, for example, the following substituted heterocyclic group containing a nitrogen atom (specific example group G2B1), the following substituted heterocyclic group containing an oxygen atom (specific example group G2B2), the following substituted heterocyclic group containing a sulfur atom (specific example group G2B3), and the following group in which one or more hydrogen atoms of the monovalent heterocyclic group derived from the ring structures represented by each of the following general formulas (TEMP-16) to (TEMP-33) are substituted by a substituent (specific example group G2B4).

### Unsubstituted Heterocyclic Group Containing a Nitrogen Atom (Specific Example Group G2A1):

a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

### Unsubstituted Heterocyclic Group Containing an Oxygen Atom (Specific Example Group G2A2):

a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

### Unsubstituted Heterocyclic Group Containing a Sulfur Atom (Specific Example Group G2A3):

a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).

Monovalent heterocyclic group derived by removing one hydrogen atom from the ring structures represented by each of the following general formulas (TEMP-16) to (TEMP-33) (specific example group G2A4):

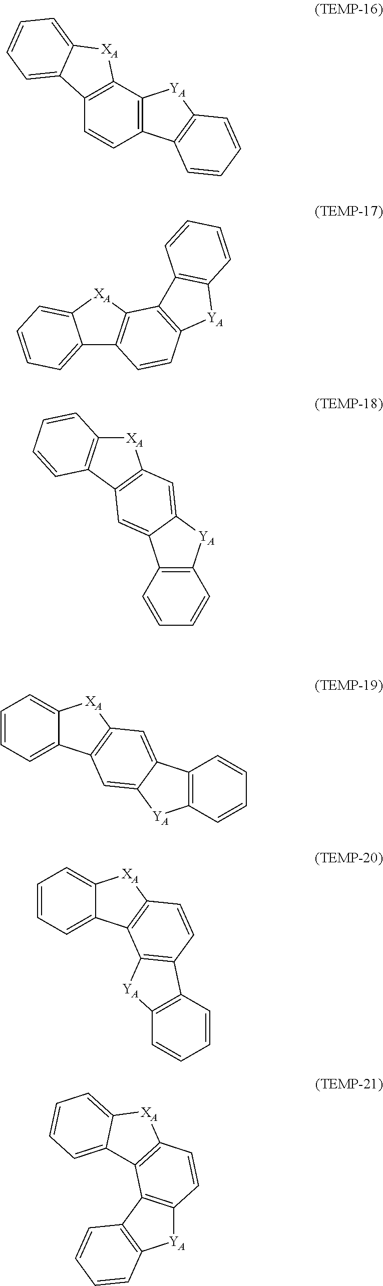

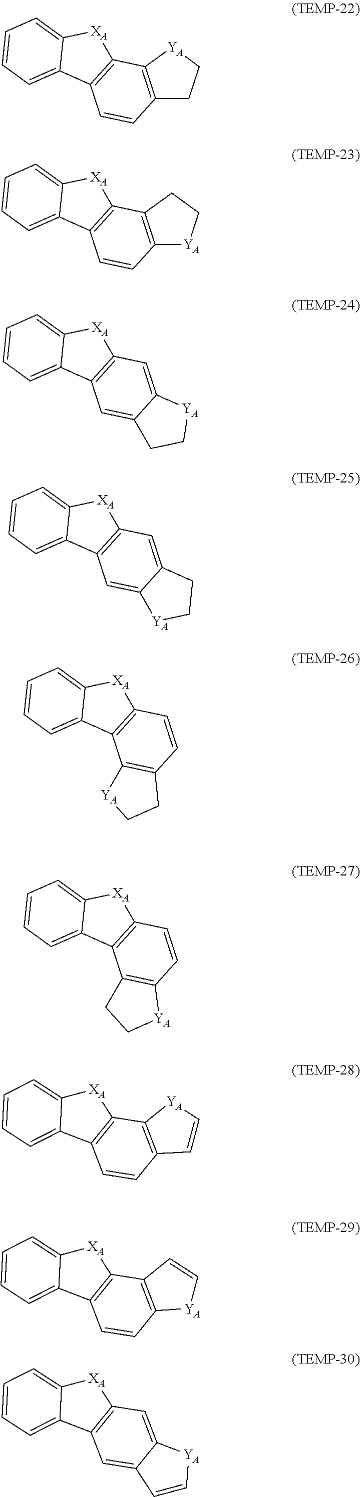

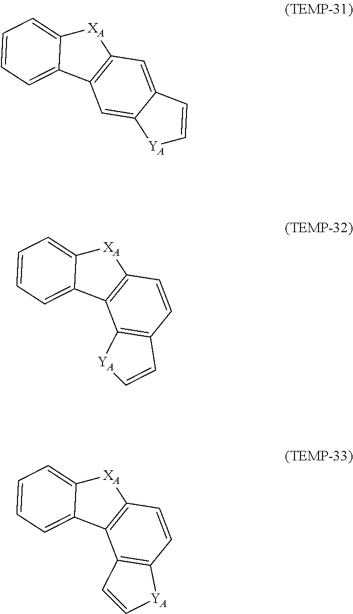

In the general formulas (TEMP-16) to (TEMP-33), X_{A} and Y_{A} are independently an oxygen atom, a sulfur atom, NH, or CH₂. Provided that at least one of X_{A} and Y_{A} is an oxygen atom, a sulfur atom, or NH.

In the general formulas (TEMP-16) to (TEMP-33), when at least one of X_{A} and Y_{A} is NH or CH₂, the monovalent heterocyclic group derived from the ring structures represented by each of the general formulas (TEMP-16) to (TEMP-33) includes a monovalent group derived by removing one hydrogen atom from these NH or CH₂.

### Substituted Heterocyclic Group Containing a Nitrogen Atom (Specific Example Group G2B1):

a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenylyltriazinyl group,
a diphenyltriazinyl group,
a phenylquinazolinyl group, and
a biphenylylquinazolinyl group.

### Substituted Heterocyclic Group Containing an Oxygen Atom (Specific Example Group G2B2):

a phenyldibenzofuranyl group,
a methyldibenzofuranyl group,
a t-butyldibenzofuranyl group, and
a monovalent residue of spiro[9H-xanthene-9,9′-[9H]fluorene].

### Substituted Heterocyclic Group Containing a Sulfur Atom (Specific Example Group G2B3):

a phenyldibenzothiophenyl group,
a methyldibenzothiophenyl group,
a t-butyldibenzothiophenyl group, and
a monovalent residue of spiro[9H-thioxanthene-9,9′-[9H]fluorene].
   Group in which One or More Hydrogen Atoms of the Monovalent Heterocyclic Group Derived from the Ring Structures Represented by Each of the Following General Formulas (TEMP-16) to (TEMP-33) are Substituted by a Substituent (Specific Example Group G2B4):

The “one or more hydrogen atoms of the monovalent heterocyclic group” means one or more hydrogen atoms selected from hydrogen atoms bonded with ring carbon atoms of the monovalent heterocyclic group, a hydrogen atom bonded with a nitrogen atom when at least one of X_{A} and Y_{A} is NH, and hydrogen atoms of a methylene group when one of X_{A} and Y_{A} is CH₂.

### “Substituted or Unsubstituted Alkyl Group”

Specific examples of the “substituted or unsubstituted alkyl group” (specific example group G3) described in this specification include the following unsubstituted alkyl groups (specific example group G3A) and the following substituted alkyl groups (specific example group G3B). (Here, the unsubstituted alkyl group refers to the case where the “substituted or unsubstituted alkyl group” is an “alkyl group unsubstituted by a substituent”, and the substituted alkyl group refers to the case where the “substituted or unsubstituted alkyl group” is an “alkyl group substituted by a substituent”). In this specification, in the case where simply referred as an “alkyl group” includes both the “unsubstituted alkyl group” and the “substituted alkyl group.”

The “substituted alkyl group” means a group in which one or more hydrogen atoms in the “unsubstituted alkyl group” are substituted by a substituent. Specific examples of the “substituted alkyl group” include groups in which one or more hydrogen atoms in the following “unsubstituted alkyl group” (specific example group G3A) are substituted by a substituent, the following substituted alkyl group (specific example group G3B), and the like. In this specification, the alkyl group in the “unsubstituted alkyl group” means a linear alkyl group. Thus, the “unsubstituted alkyl group” includes a straight-chain “unsubstituted alkyl group” and a branched-chain “unsubstituted alkyl group”. It should be noted that the examples of the “unsubstituted alkyl group” and the examples of the “substituted alkyl group” enumerated in this specification are mere examples, and the “substituted alkyl group” described in this specification includes a group in which hydrogen atom of the alkyl group itself in the “substituted alkyl group” of the specific example group G3B is further substituted by a substituent, and a group in which hydrogen atom of a substituent in the “substituted alkyl group” of the specific example group G3B is further substituted by a substituent.

### Unsubstituted Alkyl Group (Specific Example Group G3A):

a methyl group,
an ethyl group,
a n-propyl group,
an isopropyl group,
a n-butyl group,
an isobutyl group,
a s-butyl group, and
a t-butyl group.

### Substituted Alkyl Group (Specific Example Group G3B):

a heptafluoropropyl group (including isomers),
a pentafluoroethyl group,
a 2,2,2-trifluoroethyl group, and
a trifluoromethyl group.

### “Substituted or Unsubstituted Alkenyl Group”

Specific examples of the “substituted or unsubstituted alkenyl group” described in this specification (specific example group G4) include the following unsubstituted alkenyl group (specific example group G4A), the following substituted alkenyl group (specific example group G4B), and the like. (Here, the unsubstituted alkenyl group refers to the case where the “substituted or unsubstituted alkenyl group“is a” alkenyl group unsubstituted by a substituent”, and the “substituted alkenyl group” refers to the case where the “substituted or unsubstituted alkenyl group” is a “alkenyl group substituted by a substituent.”). In this specification, in the case where simply referred as an “alkenyl group” includes both the “unsubstituted alkenyl group” and the “substituted alkenyl group.”

The “substituted alkenyl group” means a group in which one or more hydrogen atoms in the “unsubstituted alkenyl group” are substituted by a substituent. Specific examples of the “substituted alkenyl group” include a group in which the following “unsubstituted alkenyl group” (specific example group G4A) has a substituent, the following substituted alkenyl group (specific example group G4B), and the like. It should be noted that the examples of the “unsubstituted alkenyl group” and the examples of the “substituted alkenyl group” enumerated in this specification are mere examples, and the “substituted alkenyl group” described in this specification includes a group in which a hydrogen atom of the alkenyl group itself in the “substituted alkenyl group” of the specific example group G4B is further substituted by a substituent, and a group in which a hydrogen atom of a substituent in the “substituted alkenyl group” of the specific example group G4B is further substituted by a substituent.

### Unsubstituted Alkenyl Group (Specific Example Group G4A):

a vinyl group,
an allyl group,
a 1-butenyl group,
a 2-butenyl group, and
a 3-butenyl group.

### Substituted Alkenyl Group (Specific Example Group G4B):

a 1,3-butanedienyl group,
a 1-methylvinyl group,
a 1-methylallyl group,
a 1,1-dimethylallyl group,
a 2-methylally group, and
a 1,2-dimethylallyl group.

### “Substituted or Unsubstituted Alkynyl Group”

Specific examples of the “substituted or unsubstituted alkynyl group” described in this specification (specific example group G5) include the following unsubstituted alkynyl group (specific example group G5A) and the like. (Here, the unsubstituted alkynyl group refers to the case where the “substituted or unsubstituted alkynyl group” is an “alkynyl group unsubstituted by a substituent”). In this specification, in the case where simply referred as an “alkynyl group” includes both the “unsubstituted alkynyl group” and the “substituted alkynyl group.”

The “substituted alkynyl group” means a group in which one or more hydrogen atoms in the “unsubstituted alkynyl group” are substituted by a substituent. Specific examples of the “substituted alkynyl group” include a group in which one or more hydrogen atoms in the following “unsubstituted alkynyl group” (specific example group G5A) are substituted by a substituent, and the like.

### Unsubstituted Alkynyl Group (Specific Example Group G5A):

an ethynyl group.

### “Substituted or Unsubstituted Cycloalkyl Group”

Specific examples of the “substituted or unsubstituted cycloalkyl group” described in this specification (specific example group G6) include the following unsubstituted cycloalkyl group (specific example group G6A), the following substituted cycloalkyl group (specific example group G6B), and the like. (Here, the unsubstituted cycloalkyl group refers to the case where the “substituted or unsubstituted cycloalkyl group“is a” cycloalkyl group unsubstituted by a substituent”, and the substituted cycloalkyl group refers to the case where the “substituted or unsubstituted cycloalkyl group” is a “cycloalkyl group substituted by a substituent”). In this specification, in the case where simply referred as a “cycloalkyl group” includes both the “unsubstituted cycloalkyl group” and the “substituted cycloalkyl group.”

The “substituted cycloalkyl group” means a group in which one or more hydrogen atoms in the “unsubstituted cycloalkyl group” are substituted by a substituent. Specific examples of the “substituted cycloalkyl group” include a group in which one or more hydrogen atoms in the following “unsubstituted cycloalkyl group” (specific example group G6A) are substituted by a substituent, and examples of the following substituted cycloalkyl group (specific example group G6B), and the like. It should be noted that the examples of the “unsubstituted cycloalkyl group” and the examples of the “substituted cycloalkyl group” enumerated in this specification are mere examples, and the “substituted cycloalkyl group” in this specification includes a group in which one or more hydrogen atoms bonded with the carbon atom of the cycloalkyl group itself in the “substituted cycloalkyl group” of the specific example group G6B are substituted by a substituent, and a group in which a hydrogen atom of a substituent in the “substituted cycloalkyl group” of specific example group G6B is further substituted by a substituent.

### Unsubstituted Cycloalkyl Group (Specific Example Group G6A):

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

### Substituted Cycloalkyl Group (Specific Example Group G6B):

a 4-methylcyclohexyl group.
   “Group represented by —Si(R₉₀₁)(R₉₀₂)(R₉₀₃)”

Specific examples of the group represented by —Si(R₉₀₁)(R₉₀₂)(R₉₀₃) described in this specification (specific example group G7) include:

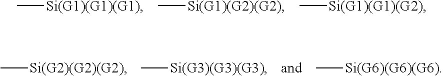

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —Si(G1)(G1)(G1) are the same or different.

Plural G2's in —Si(G1)(G2)(G2) are the same or different.

Plural G1's in —Si(G1)(G1)(G2) are the same or different.

Plural G2's in —Si(G2)(G2)(G2) are be the same or different.

Plural G3's in —Si(G3)(G3)(G3) are the same or different.

Plural G6's in —Si(G6)(G6)(G6) are be the same or different.

### “Group Represented by —O—(R₉₀₄)”

Specific examples of the group represented by —O—(R₉₀₄) in this specification (specific example group G8) include:

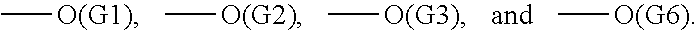

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

### “Group Represented by —S—(R₉₀₅)”

Specific examples of the group represented by —S—(R₉₀₅) in this specification (specific example group G9) include:

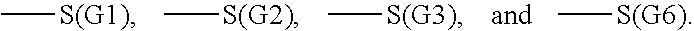

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

“Group represented by —N(R₉₀₆)(R₉₀₇)” Specific examples of the group represented by —N(R₉₀₆)(R₉₀₇) in this specification (specific example group G10) include:

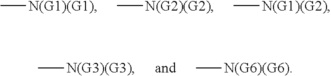

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —N(G1)(G1) are the same or different.

Plural G2's in —N(G2)(G2) are the same or different.

Plural G3's in —N(G3)(G3) are the same or different.

Plural G6's in —N(G6)(G6) are the same or different.

### “Halogen Atom”

Specific examples of the “halogen atom” described in this specification (specific example group G11) include a fluorine atom, a chlorine atom, a bromine atom, an iodine atom, and the like.

### “Substituted or Unsubstituted Fluoroalkyl Group”

The “substituted or unsubstituted fluoroalkyl group” described in this specification is a group in which at least one hydrogen atom bonded with a carbon atom constituting the alkyl group in the “substituted or unsubstituted alkyl group” is substituted by a fluorine atom, and includes a group in which all hydrogen atoms bonded with a carbon atom constituting the alkyl group in the “substituted or unsubstituted alkyl group” are substituted by a fluorine atom (a perfluoro group). The number of carbon atoms of the “unsubstituted fluoroalkyl group” is 1 to 50, preferably 1 to 30, more preferably 1 to 18, unless otherwise specified in this specification. The “substituted fluoroalkyl group” means a group in which one or more hydrogen atoms of the “fluoroalkyl group” are substituted by a substituent. The “substituted fluoroalkyl group” described in this specification also includes a group in which one or more hydrogen atoms bonded with a carbon atom of the alkyl chains in the “substituted fluoroalkyl group” are further substituted by a substituent, and a group in which one or more hydrogen atom of a substituent in the “substituted fluoroalkyl group” are further substituted by a substituent. Specific examples of the “unsubstituted fluoroalkyl group” include a group in which one or more hydrogen atoms in the “alkyl group” (specific group G3) are substituted by a fluorine atom, and the like.

### “Substituted or Unsubstituted Haloalkyl Group”

The “substituted or unsubstituted haloalkyl group” described in this specification is a group in which at least one hydrogen atom bonded with a carbon atom constituting the alkyl group in the “substituted or unsubstituted alkyl group” is substituted by a halogen atom, and also includes a group in which all hydrogen atoms bonded with a carbon atom constituting the alkyl group in the “substituted or unsubstituted alkyl group” are substituted by a halogen atom. The number of carbon atoms of the “unsubstituted haloalkyl group” is 1 to 50, preferably 1 to 30, more preferably 1 to 18, unless otherwise specified in this specification. The “substituted haloalkyl group” means a group in which one or more hydrogen atoms of the “haloalkyl group” are substituted by a substituent. The “substituted haloalkyl group” described in this specification also includes a group in which one or more hydrogen atoms bonded with a carbon atom of the alkyl chain in the “substituted haloalkyl group” are further substituted by a substituent, and a group in which one or more hydrogen atoms of a substituent in the “substituted haloalkyl group” are further substituted by a substituent. Specific examples of the “unsubstituted haloalkyl group” include a group in which one or more hydrogen atoms in the “alkyl group” (specific example group G3) are substituted by a halogen atom, and the like. A haloalkyl group is sometimes referred to as an alkyl halide group.

### “Substituted or Unsubstituted Alkoxy Group”

Specific examples of the “substituted or unsubstituted alkoxy group” described in this specification include a group represented by —O(G3), wherein G3 is the “substituted or unsubstituted alkyl group” described in the specific example group G3. The number of carbon atoms of the “unsubstituted alkoxy group” is 1 to 50, preferably 1 to 30, more preferably 1 to 18, unless otherwise specified in this specification.

### “Substituted or Unsubstituted Alkylthio Group”

Specific examples of the “substituted or unsubstituted alkylthio group” described in this specification include a group represented by —S(G3), wherein G3 is the “substituted or unsubstituted alkyl group” described in the specific example group G3. The number of carbon atoms of the “unsubstituted alkylthio group” is 1 to 50, preferably 1 to 30, more preferably 1 to 18, unless otherwise specified in this specification.

### “Substituted or Unsubstituted Aryloxy Group”

Specific examples of the “substituted or unsubstituted aryloxy group” described in this specification include a group represented by —O(G1), wherein G1 is the “substituted or unsubstituted aryl group” described in the specific example group G1. The number of ring carbon atoms of the “unsubstituted aryloxy group” is 6 to 50, preferably 6 to 30, more preferably 6 to 18, unless otherwise specified in this specification.

### “Substituted or Unsubstituted Arylthio Group”

Specific examples of the “substituted or unsubstituted arylthio group” described in this specification include a group represented by —S(G1), wherein G1 is a “substituted or unsubstituted aryl group” described in the specific example group G1. The number of ring carbon atoms of the “unsubstituted arylthio group” is 6 to 50, preferably 6 to 30, more preferably 6 to 18, unless otherwise specified in this specification.

### “Substituted or Unsubstituted Trialkylsilyl Group”

Specific examples of the “trialkylsilyl group” described in this specification include a group represented by —Si(G3)(G3)(G3), where G3 is the “substituted or unsubstituted alkyl group” described in the specific example group G3. Plural G3's in —Si(G3)(G3)(G3) are the same or different. The number of carbon atoms in each alkyl group of the “trialkylsilyl group” is 1 to 50, preferably 1 to 20, more preferably 1 to 6, unless otherwise specified in this specification.

### “Substituted or Unsubstituted Aralkyl Group”

Specific examples of the “substituted or unsubstituted aralkyl group” described in this specification is a group represented by -(G3)-(G1), wherein G3 is the “substituted or unsubstituted alkyl group” described in the specific example group G3, and G1 is the “substituted or unsubstituted aryl group” described in the specific example group G1. Therefore, the “aralkyl group” is a group in which a hydrogen atom of the “alkyl group” is substituted by an “aryl group” as a substituent, and is one form of the “substituted alkyl group.” The “unsubstituted aralkyl group” is the “unsubstituted alkyl group” substituted by the “unsubstituted aryl group”, and the number of carbon atoms of the “unsubstituted aralkyl group” is 7 to 50, preferably 7 to 30, more preferably 7 to 18, unless otherwise specified in this specification.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, and the like.

Unless otherwise specified in this specification, examples of the substituted or unsubstituted aryl group described in this specification preferably include a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, 9,9-dimethylfluorenyl group, 9,9-diphenylfluorenyl group, and the like.

Unless otherwise specified in this specification, examples of the substituted or unsubstituted heterocyclic groups described in this specification preferably include a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like.

In this specification, the carbazolyl group is specifically each of the following groups, unless otherwise specified in this specification.

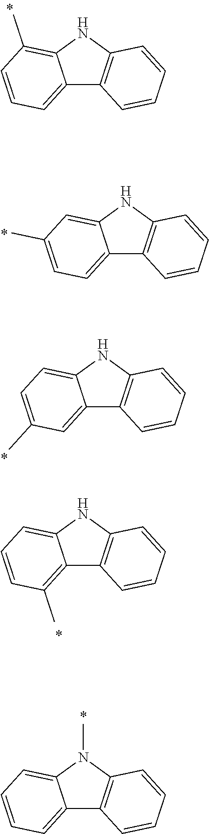

In this specification, the (9-phenyl)carbazolyl group is specifically any of the following groups, unless otherwise specified in this specification.

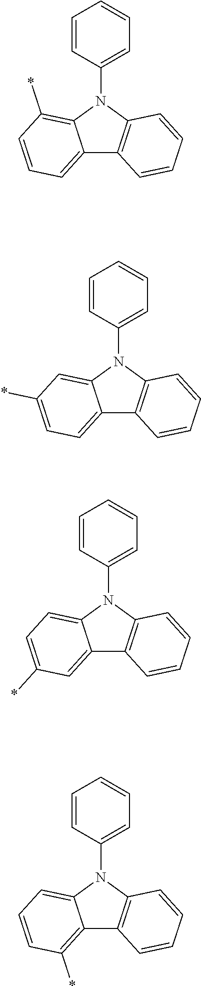 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the general formulas (TEMP-Cz1) to (TEMP-Cz9), * represents a bonding site.

In this specification, the dibenzofuranyl group and the dibenzothiophenyl group are specifically any of the following groups, unless otherwise specified in this specification.

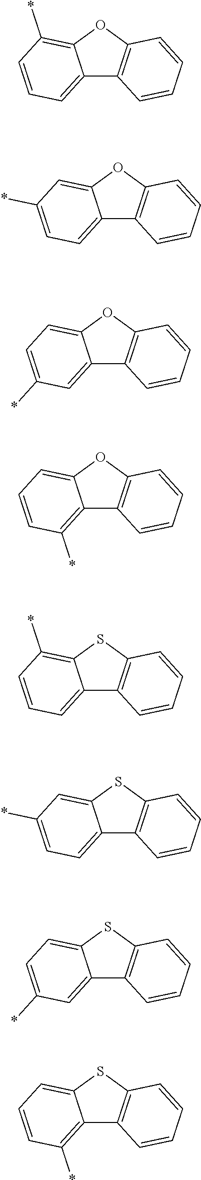

In the general formulas (TEMP-34) to (TEMP-41), * represents a bonding site.

The substituted or unsubstituted alkyl group described in this specification is preferably a methyl group, an ethyl group, a propyl group, an isopropyl group, a n-butyl group, an isobutyl group, a t-butyl group, or the like, unless otherwise specified in this specification.

### “Substituted or Unsubstituted Arylene Group”

The “substituted or unsubstituted arylene group” described in this specification is a divalent group derived by removing one hydrogen atom on the aryl ring of the “substituted or unsubstituted aryl group”, unless otherwise specified. Specific examples of the “substituted or unsubstituted arylene group” (specific example group G12) include a divalent group derived by removing one hydrogen atom on the aryl ring of the “substituted or unsubstituted aryl group” described in the specific example group G1, and the like.

### “Substituted or Unsubstituted Divalent Heterocyclic Group”

The “substituted or unsubstituted divalent heterocyclic group” described in this specification is a divalent group derived by removing one hydrogen atom on the heterocycle of the “substituted or unsubstituted heterocyclic group”, unless otherwise specified. Specific examples of the “substituted or unsubstituted divalent heterocyclic group” (specific example group G13) include a divalent group derived by removing one hydrogen atom on the heterocycle of the “substituted or unsubstituted heterocyclic group” described in the specific example group G2, and the like.

### “Substituted or Unsubstituted Alkylene Group”

The “substituted or unsubstituted alkylene group” described in this specification is a divalent group derived by removing one hydrogen atom on the alkyl chain of the “substituted or unsubstituted alkyl group”, unless otherwise specified. Specific examples of the “substituted or unsubstituted alkylene group” (specific example group G14) include a divalent group derived by removing one hydrogen atom on the alkyl chain of the “substituted or unsubstituted alkyl group” described in the specific example group G3, and the like.

The substituted or unsubstituted arylene group described in this specification is preferably any group of the following general formulas (TEMP-42) to (TEMP-68), unless otherwise specified in this specification.

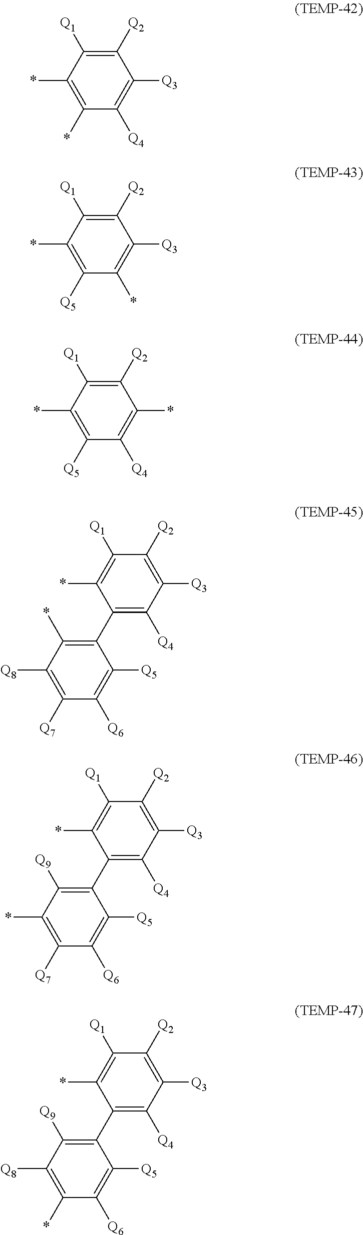

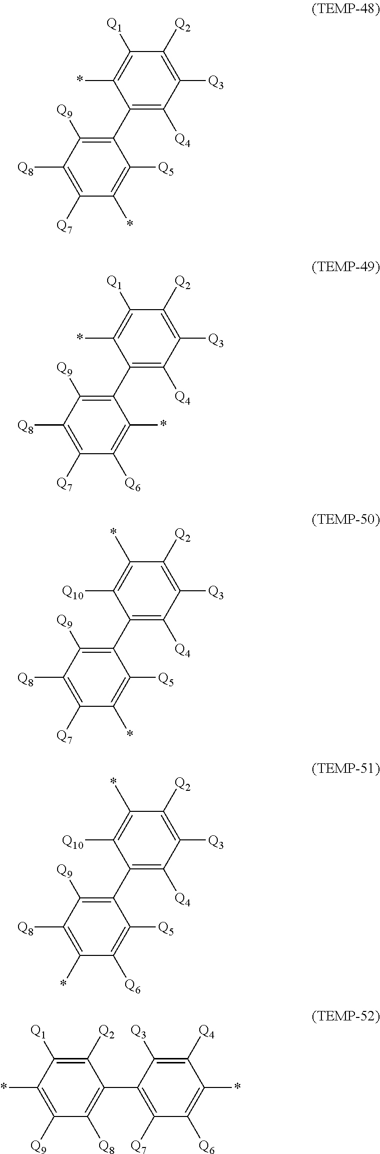

In the general formulas (TEMP-42) to (TEMP-52), Q₁ to Q₁₀ are independently a hydrogen atom or a substituent.

In the general formulas (TEMP-42) to (TEMP-52), * represents a bonding site.

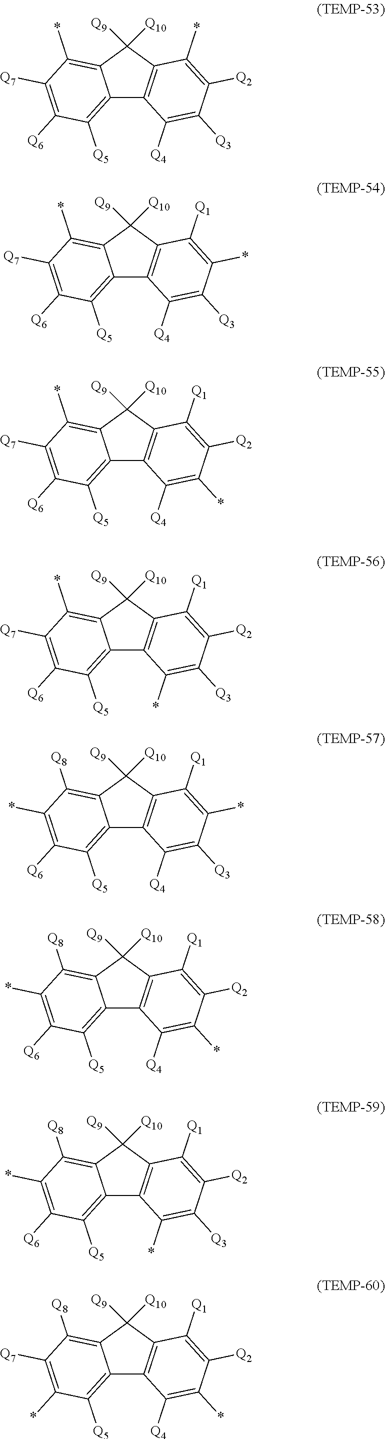

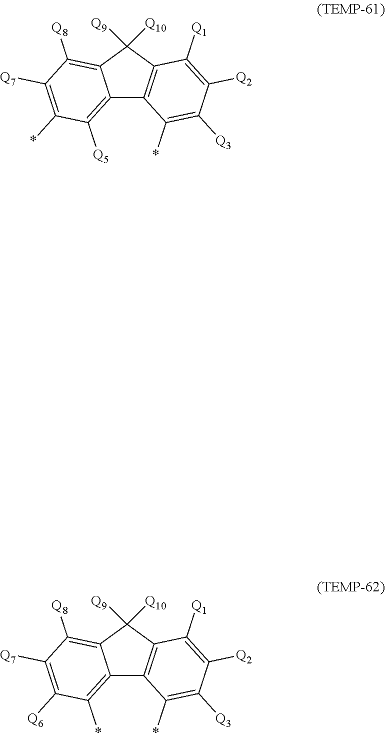

In the general formulas (TEMP-53) to (TEMP-62), Q₁ to Q₁₀ are independently a hydrogen atom or a substituent.

Q₉ and Q₁₀ may be bonded with each other via a single bond to form a ring.

In the general formulas (TEMP-53) to (TEMP-62), * represents a bonding site.

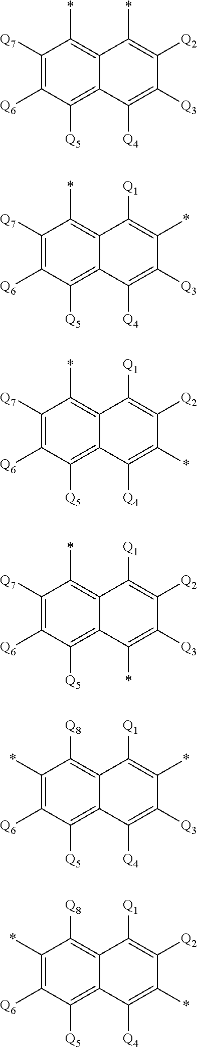

In the general formulas (TEMP-63) to (TEMP-68), Q₁ to Q₈ are independently a hydrogen atom or a substituent.

In the general formulas (TEMP-63) to (TEMP-68), * represents a bonding site.

The substituted or unsubstituted divalent heterocyclic group described in this specification is preferably any group of the following general formulas (TEMP-69) to (TEMP-102), unless otherwise specified in this specification.

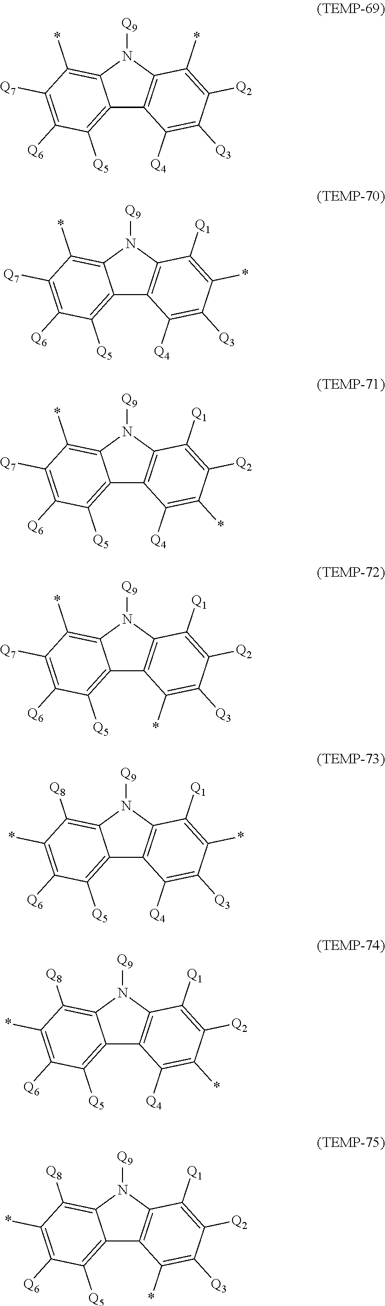

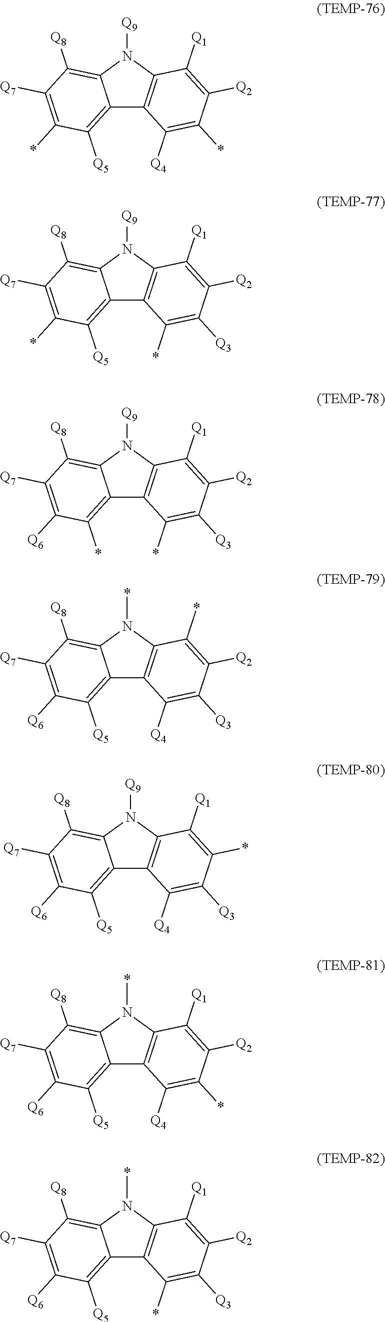

In the general formulas (TEMP-69) to (TEMP-82), Q₁ to Q₉ are independently a hydrogen atom or a substituent.

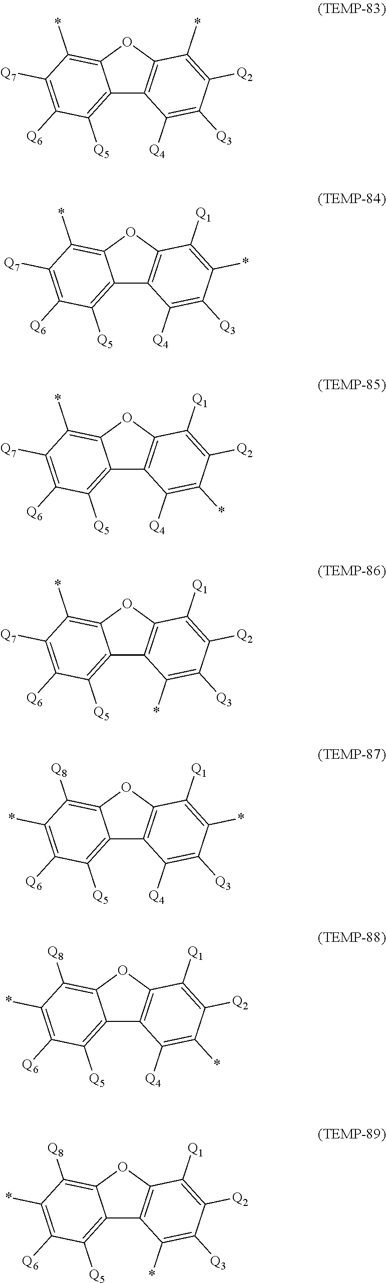

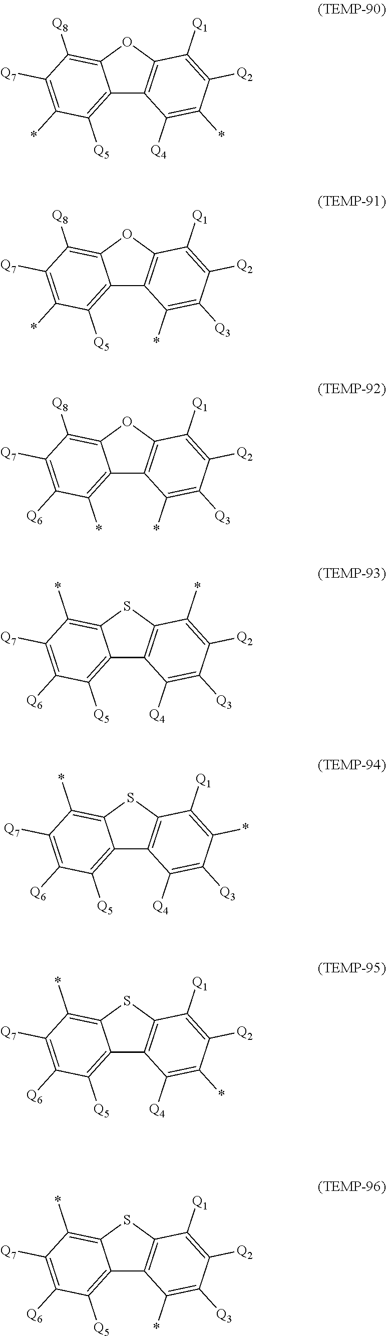

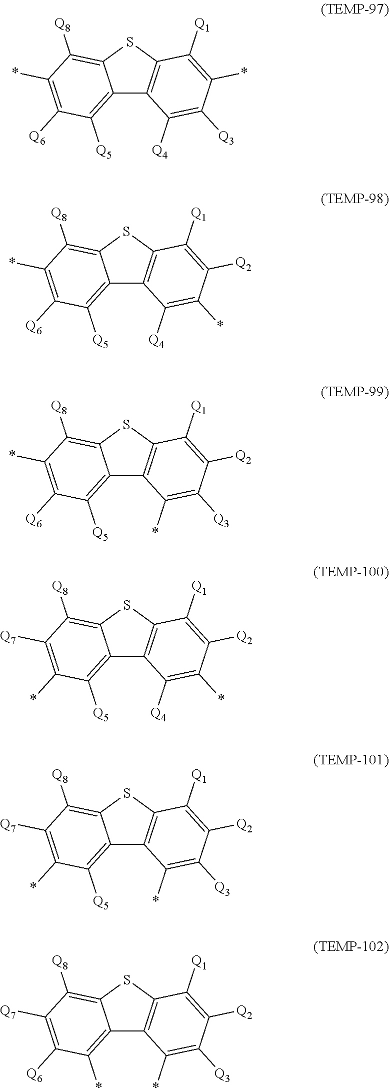

In the general formulas (TEMP-83) to (TEMP-102), Q₁ to Q₈ are independently a hydrogen atom or a substituent.

The above is the explanation of the “Substituent described in this specification.”

“the Case where Bonded with Each Other to Form a Ring”

In this specification, the case where “one or more sets of adjacent two or more form a substituted or unsubstituted monocycle by bonding with each other, form a substituted or unsubstituted fused ring by bonding with each other, or do not bond with each other” means the case where “one or more sets of adjacent two or more form a substituted or unsubstituted monocycle by bonding with each other”; the case where “one or more sets of adjacent two or more form a substituted or unsubstituted fused ring by bonding with each other”; and the case where “one or more sets of adjacent two or more do not bond with each other.”

The case where “one or more sets of adjacent two or more form a substituted or unsubstituted monocycle by bonding with each other” and the case where “one or more sets of adjacent two or more form a substituted or unsubstituted fused ring by bonding with each other” in this specification (these cases may be collectively referred to as “the case where forming a ring by bonding with each other”) will be described below. The case of an anthracene compound represented by the following general formula (TEMP-103) in which the mother skeleton is an anthracene ring will be described as an example.

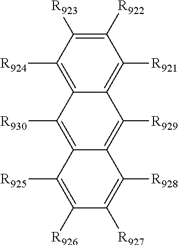

For example, in the case where “one or more sets of adjacent two or more among R₉₂₁ to R₉₃₀ form a ring by bonding with each other”, the one set of adjacent two includes a pair of R₉₂₁ and R₉₂₂, a pair of R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₆, a pair of R₉₂₆ and R₉₂₇, a pair of R₉₂₇ and R₉₂₈, a pair of R₉₂₈ and R₉₂₉, and a pair of R₉₂₉ and R₉₂₁.

The “one or more sets” means that two or more sets of the adjacent two or more sets may form a ring at the same time. For example, R₉₂₁ and R₉₂₂ form a ring Q_{A} by bonding with each other, and at the same, time R₉₂₅ and R₉₂₆ form a ring Q_{B} by bonding with each other, the anthracene compound represented by the general formula (TEMP-103) is represented by the following general formula (TEMP-104).

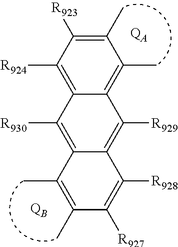

The case where the “set of adjacent two or more” form a ring includes not only the case where the set (pair) of adjacent “two” is bonded with as in the above-mentioned examples, but also the case where the set of adjacent “three or more” are bonded with each other. For example, it means the case where R₉₂₁ and R₉₂₂ form a ring Q_{A} by bonding with each other, and R₉₂₂ and R₉₂₃ form a ring Q_{C} by bonding with each other, and adjacent three (R₉₂₁, R₉₂₂ and R₉₂₃) form rings by bonding with each other and together fused to the anthracene mother skeleton. In this case, the anthracene compound represented by the general formula (TEMP-103) is represented by the following general formula (TEMP-105). In the following general formula (TEMP-105), the ring Q_{A} and the ring Q_{C} share R₉₂₂.

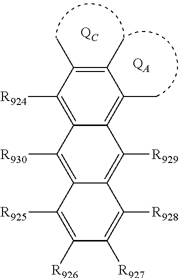

The “monocycle” or “fused ring” formed may be a saturated ring or an unsaturated ring, as a structure of the formed ring alone. Even when the “one pair of adjacent two” forms a “monocycle” or a “fused ring”, the “monocycle” or the “fused ring” may form a saturated ring or an unsaturated ring. For example, the ring Q_{A} and the ring Q_{B} formed in the general formula (TEMP-104) are independently a “monocycle” or a “fused ring.” The ring Q_{A} and the ring Q_{C} formed in the general formula (TEMP-105) are “fused ring.” The ring Q_{A} and ring Q_{C} of the general formula (TEMP-105) are fused ring by fusing the ring Q_{A} and the ring Q_{C} together. When the ring Q_{A} of the general formula (TMEP-104) is a benzene ring, the ring Q_{A} is a monocycle. When the ring Q_{A} of the general formula (TMEP-104) is a naphthalene ring, the ring Q_{A} is a fused ring.

The “unsaturated ring” includes, in addition to an aromatic hydrocarbon ring and an aromatic heterocycle, an aliphatic hydrocarbon ring with an unsaturated bond, i.e., double and/or triple bonds in the ring structure (e.g., cyclohexene, cyclohexadiene, etc.), and a non-aromatic heterocycle with an unsaturated bond (e.g., dihydropyran, imidazoline, pyrazoline, quinolizine, indoline, isoindoline, etc.). The “saturated ring” includes an aliphatic hydrocarbon ring without an unsaturated bond and a non-aromatic heterocycle without ab unsaturated bond.

Specific examples of the aromatic hydrocarbon ring include a structure in which the group listed as a specific example in the specific example group G1 is terminated by a hydrogen atom.

Specific examples of the aromatic heterocycle include a structure in which the aromatic heterocyclic group listed as a specific example in the example group G2 is terminated by a hydrogen atom.

Specific examples of the aliphatic hydrocarbon ring include a structure in which the group listed as a specific example in the specific example group G6 is terminated by a hydrogen atom.

The term “to form a ring” means forming a ring only with plural atoms of the mother skeleton, or with plural atoms of the mother skeleton and one or more arbitrary atoms in addition. For example, the ring Q_{A} shown in the general formula (TEMP-104), which is formed by bonding R₉₂₁ and R₉₂₂ with each other, is a ring formed from the carbon atom of the anthracene skeleton with which R₉₂₁ is bonded, the carbon atom of the anthracene skeleton with which R₉₂₂ is bonded, and one or more arbitrary atoms. For example, in the case where the ring Q_{A} is formed with R₉₂₁ and R₉₂₂, when a monocyclic unsaturated ring is formed with the carbon atom of the anthracene skeleton with which R₉₂₁ is bonded, the carbon atom of the anthracene skeleton with which R₉₂₂ is bonded, and four carbon atoms, the ring formed with R₉₂₁ and R₉₂₂ is a benzene ring.

Here, the “arbitrary atom” is preferably at least one atom selected from the group consisting of a carbon atom, a nitrogen atom, an oxygen atom, and a sulfur atom, unless otherwise specified in this specification. In the arbitrary atom (for example, a carbon atom or a nitrogen atom), a bond which does not form a ring may be terminated with a hydrogen atom or the like, or may be substituted with “arbitrary substituent” described below. When an arbitrary atom other than a carbon atom is contained, the ring formed is a heterocycle.

The number of “one or more arbitrary atom(s)” constituting a monocycle or a fused ring is preferably 2 or more and 15 or less, more preferably 3 or more and 12 or less, and still more preferably 3 or more and 5 or less, unless otherwise specified in this specification.

The “monocycle” is preferable among the “monocycle” and the “fused ring”, unless otherwise specified in this specification.

The “unsaturated ring” is preferable among the “saturated ring” and the “unsaturated ring”, unless otherwise specified in this specification.

Unless otherwise specified in this specification, the “monocycle” is preferably a benzene ring.

Unless otherwise specified in this specification, the “unsaturated ring” is preferably a benzene ring.

Unless otherwise specified in this specification, when “one or more sets of adjacent two or more” are “bonded with each other to form a substituted or unsubstituted monocycle” or “bonded with each other to form a substituted or unsubstituted fused ring”, this specification, one or more sets of adjacent two or more are preferably bonded with each other to form a substituted or unsubstituted “unsaturated ring” from plural atoms of the mother skeleton and one or more and 15 or less atoms which is at least one kind selected from a carbon atom, a nitrogen atom, an oxygen atom, and a sulfur atom.

The substituent in the case where the above-mentioned “monocycle” or “fused ring” has a substituent is, for example, an “arbitrary substituent” described below. Specific examples of the substituent which the above-mentioned “monocycle” or “fused ring” has include the substituent described above in the “Substituent described in this specification” section.

The substituent in the case where the above-mentioned “saturated ring” or “unsaturated ring” has a substituent is, for example, an “arbitrary substituent” described below. Specific examples of the substituent which the above-mentioned “monocycle” or “fused ring” has include the substituent described above in the “Substituent described in this specification” section.

The foregoing describes the case where “one or more sets of adjacent two or more form a substituted or unsubstituted monocycle by bonding with each other” and the case where “one or more sets of adjacent two or more form a substituted or unsubstituted fused ring by bonding with each other “(the case where “forming a ring by bonding with each other”).

### Substituent in the Case of “Substituted or Unsubstituted”

In one embodiment in this specification, the substituent (in this specification, sometimes referred to as an “arbitrary substituent”) in the case of “substituted or unsubstituted” is, for example, a group selected from the group consisting of:
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,

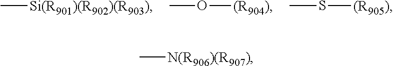

a halogen atom, a cyano group, a nitro group,
an unsubstituted aryl group including 6 to 50 ring carbon atoms, and
an unsubstituted heterocyclic group including 5 to 50 ring atoms,
wherein, R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted heterocyclic group including 5 to 50 ring atoms.
When two or more R₉₀₁'s are present, the two or more R₉₀₁'s may be the same or different.
When two or more R₉₀₂'s are present, the two or more R₉₀₂'s may be the same or different.
When two or more R₉₀₃'s are present, the two or more R₉₀₃'s may be the same or different.
When two or more R₉₀₄'s are present, the two or more R₉₀₄'s may be the same or different.
When two or more R₉₀₅'s are present, the two or more R₉₀₅'s may be the same or different.
When two or more R₉₀₆'s are present, the two or more R₉₀₆'s may be the same or different.
When two or more R₉₀₇'s are present, the two or more R₉₀₇'s may be the same or different.

In one embodiment, the substituent in the case of “substituted or unsubstituted” is a group selected from the group consisting of:
an alkyl group including 1 to 50 carbon atoms,
an aryl group including 6 to 50 ring carbon atoms, and
a heterocyclic group including 5 to 50 ring atoms.

In one embodiment, the substituent in the case of “substituted or unsubstituted” is a group selected from the group consisting of:
an alkyl group including 1 to 18 carbon atoms,
an aryl group including 6 to 18 ring carbon atoms, and
a heterocyclic group including 5 to 18 ring atoms.

Specific examples of each of the arbitrary substituents include specific examples of substituent described in the section “Substituent described in this specification” above.

Unless otherwise specified in this specification, adjacent arbitrary substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, more preferably form a benzene ring.

Unless otherwise specified in this specification, the arbitrary substituent may further have a substituent. The substituent which the arbitrary substituent further has is the same as that of the above-mentioned arbitrary substituent.

In this specification, the numerical range represented by “AA to BB” means the range including the numerical value AA described on the front side of “AA to BB” as the lower limit and the numerical value BB described on the rear side of “AA to BB” as the upper limit.

### [Organic EL Device]

An organic EL device according to an aspect of the present invention (hereinafter, also referred to as the “organic EL device of the present invention”) is a concept including a first organic EL device, a second organic EL device, and a third organic EL device to be described later.

When the first organic EL device of the present invention has a configuration to be described later, higher device performance can be achieved. Specifically, it is possible to achieve an organic EL device having a low driving voltage and high efficiency from a low current density to a high current density.

When the second organic EL device of the present invention has a configuration to be described later, higher device performance can be achieved. Specifically, it is possible to achieve an organic EL device having a low driving voltage and high efficiency even at a low current density.

In one embodiment, an organic EL device according to an aspect of the present invention is the first organic EL device.

In one embodiment, an organic EL device according to an aspect of the present invention is the second organic EL device.

In one embodiment, an organic EL device according to an aspect of the present invention is the third organic EL device.

### [First Organic EL Device]

The first organic EL device according to an aspect of the present invention (hereinafter, also referred to as the “first organic EL device of the present invention”) includes a cathode, an anode, and one or two or more organic layers disposed between the cathode and the anode, wherein at least one layer of the one or two or more organic layers includes a first component and a second component.

The first component is a compound represented by the formula (1) described later.

The second component is selected from the group consisting of an alkali metal, an alkali metal compound, an alkaline earth metal, an alkaline earth metal compound, a rare earth metal, a rare earth metal compound, an organic metal complex containing an alkali metal, an organic metal complex containing an alkaline earth metal, and an organic metal complex containing a rare earth metal.

Since the first organic EL device of the present invention has the above-described configuration, higher device performance can be achieved. Specifically, it is possible to achieve an organic EL device having a low driving voltage and high efficiency from a low current density to a high current density.

As understood from the definition of each component, the first component and the second component are different from each other.

Hereinafter, each configuration of the first organic EL device according to an aspect of the present invention will be described.

### (First Component)

The first component in the first organic EL device according to an aspect of the present invention is the compound represented by the following formula (1):

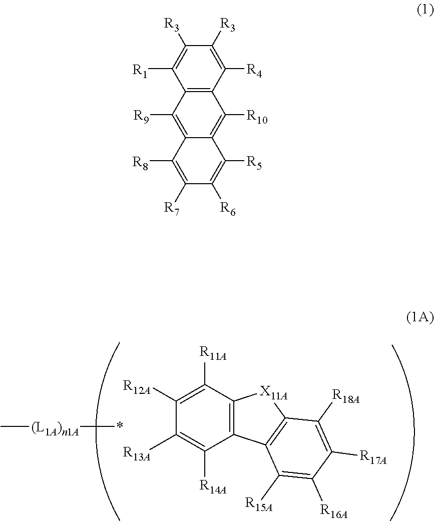

wherein in the formula (1),
at least one of R₁ to R₁₀ is a group represented by the formula (1A);
R₁ to R₁₀ that are not the group represented by the formula (1A) are independently a hydrogen atom, or a substituent A; provided that at least three of R₁ to R₁₀ are independently the group represented by the formula (1A), the substituent A, or a hydrogen atom which is a deuterium atom;
in the formula (1A),
L_{1A} is
a single bond,
a substituted or unsubstituted arylene group having 6 to 50 ring carbon atoms, or
a substituted or unsubstituted divalent heterocyclic group having 5 to 50 ring atoms; n1A is an integer of 0 to 3;
when n1A is 0, (L_{1A})_{n1A} is a single bond;
when n1A is 2 or 3, the plurality of L_{1A}'s is connected in series with each other, and a structure in parentheses is bonded to L_{1A} farthest from the anthracene skeleton; the plurality of L_{1A}'s may be the same as or different from each other;
X_{11A} is C(R_{21A})(R_{22A}), N(R_{23A}), O, or S;
one or more sets of the adjacent two or more of R_{11A} to R_{18A} form a substituted or unsubstituted single ring by bonding with each other, form a substituted or unsubstituted fused ring by bonding with each other, or do not form any of the rings;
when the substituted or unsubstituted single ring is formed, one of atoms forming the single ring is bonded to L_{1A}, or one of R_{11A} to R_{18A} that do not contribute to the formation of the single ring, and R_{21A} to R_{23A} represents a bond to L_{1A};
when the substituted or unsubstituted fused ring is formed, one of atoms forming the fused ring is bonded to L_{1A}, or one of R_{11A} to R_{18A} that do not contribute to the formation of the fused ring, and R_{21A} to R_{23A} represents a bond to L_{1A};
when the single ring and the fused ring are not formed, one of R_{11A} to R_{18A} and R_{21A} to R_{23A} represents a bond to L_{1A};
R_{11A} to R_{18A} and R_{21A} to R_{23A} that do not represent the bond to L_{1A} and that do not form any of the rings are independently a hydrogen atom or a substituent A;
when two or more groups represented by the formula (1A) are present, the two or more groups represented by the formula (1A) may be the same as or different from each other; the substituent A is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,

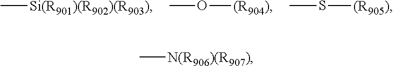

a halogen atom, a nitro group,
a substituted or unsubstituted aryl group having 6 to 50 ring carbon atoms, or
a substituted or unsubstituted monovalent heterocyclic group having 5 to 50 ring atoms;
R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more substituents A are present, the two or more substituents A may be the same as or different from each other; and
when two or more of each of R₉₀₁ to R₉₀₇ are present, the two or more of each of R₉₀₁ to R₉₀₇ may be the same as or different from each other,
provided that the compound represented by the formula (1) does not have a structure represented by the following formula (M1), a structure represented by the following formula (M2), a structure represented by the following formula (M3), and a structure represented by the following formula (M4) in the molecule thereof:

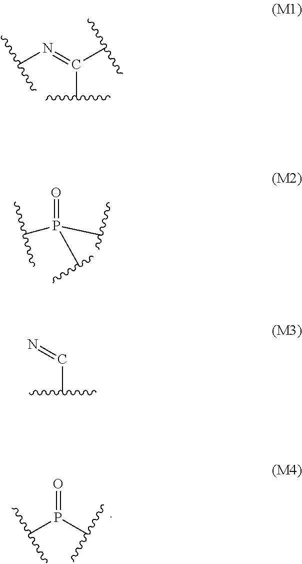

Many compounds that have the structures (electron-accepting structures) represented by the formulas (M1) to (M4) in molecules thereof and have been conventionally used as an electron-transporting material and the like of an organic EL device decrease the external quantum efficiency (EQE) of the organic EL device at a low current density.

The present inventors have found that it is advantageous, as the electron-transporting material, to use a compound (the first component in the first organic EL device according to an aspect of the present invention), which is different from the above-described conventional materials and does not have the structures represented by the formulas (M1) to (M4) in the molecule thereof, the compound having at least three predetermined groups or deuterium atoms on an anthracene skeleton, in combination with the second component to be described later, and that an organic EL device using such a material has a low driving voltage and has EQE not decreasing from a low current density to a high current density.

Although details of a mechanism in which EQE does not decrease in a low current density region are not clear, it is considered that, when a compound having any of the above-described electron-accepting structures in the molecule thereof is used, the influence of the structure on EQE is reduced in a high current density region since the influence of the voltage is large, but the influence of the structure is large in the low current density region so that carrier balance is lost due to excessive electrons, and EQE decreases. Conversely, it is considered that the carrier balance is maintained from the low current density to the high current density, and EQE does not decrease when the first component in the first organic EL device according to an aspect of the present invention that does not have the above-described electron-accepting structures in the molecule thereof is used.

In the formula (1), at least one of R₁ to R₁₀ is a group represented by the formula (1A). For example, when R₁₀ is the group represented by the formula (1A), the compound represented by the formula (1) is represented by the following formula (Ex1).

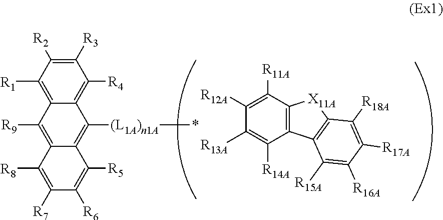

In the formula (1), at least three of R₁ to R₁₀ are independently the group represented by the formula (1A), the substituent A, or a hydrogen atom which is a deuterium atom. In other words, among R₁ to R₁₀, the number of hydrogen atoms as protium atoms is 7 or less.

Next, the phrase “when n1A is 2 or 3, the plurality of L_{1A}'S is connected in series with each other, and a structure in parentheses is bonded to L_{1A} farthest from the anthracene skeleton” will be described. For example, when R₁₀ is the group represented by the formula (1A) and n1A is 3, the compound represented by the formula (1) is represented by the following formula (Ex2).

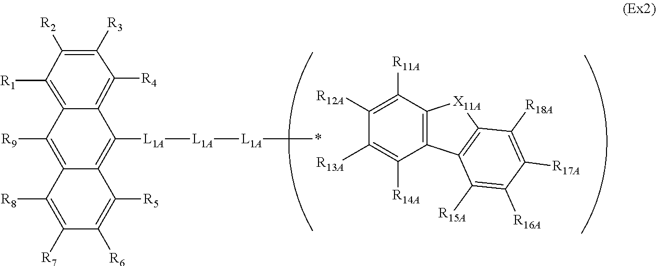

In the formula (1A), when one or more sets of the adjacent two or more of R_{11A} to R_{18A} form a substituted or unsubstituted single ring by bonding with each other, one of atoms forming the single ring is bonded to L_{1A}, or one of R_{11A} to R_{18A} that do not contribute to the formation of the single ring, and R_{21A} to R_{23A} represents a bond to L_{1A}.

The case where “one of atoms forming the single ring is bonded to L_{1A}” will be described. For example, when a set of R_{11A} and R_{12A} forms an unsubstituted benzene ring by bonding with each other, the group represented by the formula (1A) is represented by the following formula (Ex3). In the formula (Ex3), one of four carbon atoms (carbon atoms to which * is attached) forming the benzene ring is bonded to L_{1A}.

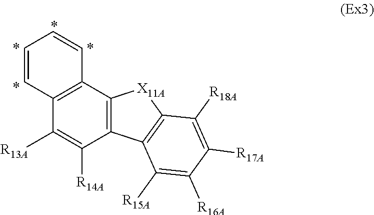

Next, the case where “one of R_{11A} to R_{18A} that do not contribute to the formation of the single ring, and R_{21A} to R_{23A} represents a bond to L_{1A}” will be described. When the set of R_{11A} and R_{12A} forms the unsubstituted benzene ring by bonding with each other, one of R_{13A} to R_{18A} and R_{21A} to R_{23A} represents the bond to L_{1A} in the above formula (Ex3).

When X_{11A} is C(R_{21A})(R_{22A}), one of R_{13A} to R_{18A} and R_{21A} to R_{22A} represents a bond to L_{1A}.
When X_{11A} is N(R_{23A}), one of R_{13A} to R_{18A} and R_{23A} represents a bond to L_{1A}.
When X_{11A} is O or S, one of R_{13A} to R_{18A} represents a bond to L_{1A}.

In the formula (1A), when one or more sets of the adjacent two or more of R_{11A} to R_{18A} form a substituted or unsubstituted fused ring by bonding with each other, one of atoms forming the fused ring is bonded to L_{1A}, or one of R_{11A} to R_{18A} that do not contribute to the formation of the fused ring, and R_{21A} to R_{23A} represents a bond to L_{1A}.

The case where “one of atoms forming the fused ring is bonded to L_{1A}” will be described. For example, when the set of R_{11A} and R_{12A} forms an unsubstituted naphthalene ring by bonding with each other, the group represented by the formula (1A) is represented by the following formulas (Ex4) to (Ex6). In the formulas (Ex4) to (Ex6), one of six carbon atoms (carbon atoms to which * is attached) forming the naphthalene ring is bonded to L_{1A}.

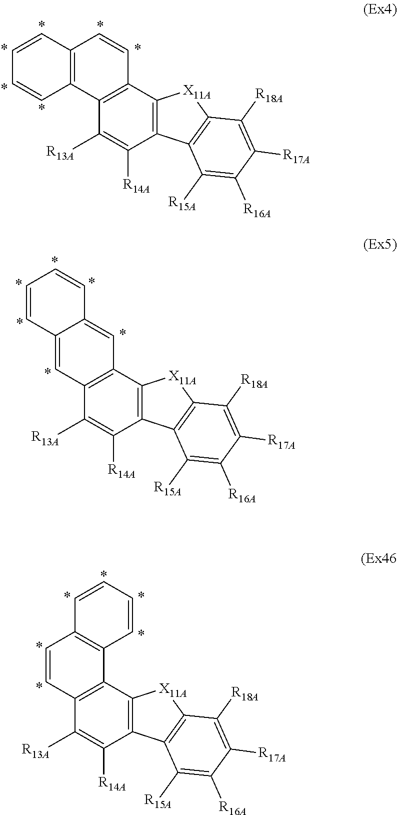

Next, the case where “one of R_{11A} to R_{18A} that do not contribute to the formation of the fused ring, and R_{21A} to R_{23A} represents a bond to L_{1A}” will be described. When the set of R_{11A} and R_{12A} forms the unsubstituted naphthalene ring by bonding with each other, in the above formulas (Ex4) to (Ex6), one of R_{13A} to R_{18A} and R_{21A} to R_{23A} represents a bond to L_{1A}.

When X_{11A} is C(R_{21A})(R_{22A}), one of R_{13A} to R_{18A} and R_{21A} to R_{22A} represents a bond to L_{1A}.

When X_{11A} is N(R_{23A}), one of R_{13A} to R_{18A} and R_{23A} represents a bond to L_{1A}.

When X_{11A} is O or S, one of R_{13A} to R_{18A} represents a bond to L_{1A}.

The phrase “does not have structures represented by the following formulas (M1) to (M4) in the molecule thereof” will be described.

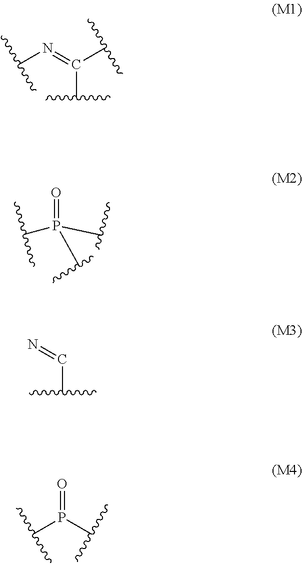

In the formulas (M1) to (M4), a wavy line terminating a bond means that some atom is present beyond the wavy line. For example, the bond may be terminated with a hydrogen atom, or may be a bond to an atom which can have a divalent or higher valence (for example, a carbon atom, a nitrogen atom, an oxygen atom, a sulfur atom, or the like).

The phrase “does not have structures represented by the formulas (M1) to (M4) in the molecule thereof” means that in any site within the molecule thereof, the structure represented by the formula (M1) is not included, the structure represented by the formula (M2) is not included, the structure represented by the formula (M3) is not included, and the structure represented by the formula (M4) is not included. For example, the structures are not included as a substituent, and the structures are not included in a basic skeleton thereof, either.

In addition, a compound having any one of the structure represented by the formula (M1), the structure represented by the formula (M2), the structure represented by the formula (M3), and the structure represented by the formula (M4) does not correspond to the first component in the first organic EL device according to an aspect of the present invention.

Examples of the substituent having the structure represented by the formula (M1) include a substituent (unsubstituted imidazolyl group) represented by the following formula (M1-1):

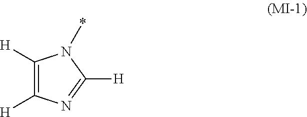 [SMILES: *n1ccnc1]

wherein in the formula (M1-1), * represents a bonding position of the substituent.

In the formula (M1-1), a carbon atom in the formula (M1) corresponds to a carbon atom at the 2-position of an imidazole skeleton of the formula (M1-1), and a nitrogen atom in the formula (M1) corresponds to a nitrogen atom at the 3-position of the imidazole skeleton of the formula (M1-1). That is, two wavy lines terminating two bonds extending from the carbon atom in the formula (M1) represent a hydrogen atom bonded to the carbon atom in the formula (M1-1) and a nitrogen atom at the 1-position of the imidazole skeleton in the formula (M1-1), and a wavy line terminating a bond extending from the nitrogen atom in the formula (M1) represents a carbon atom at the 4-position of the imidazole skeleton in the formula (M1-1).

Therefore, the first component in the first organic EL device according to an aspect of the present invention does not have an imidazolyl group and a group having an imidazolyl skeleton.

In addition, examples of a compound having the structure represented by the formula (M1) in a basic skeleton include a compound represented by the following formula (M1-2).

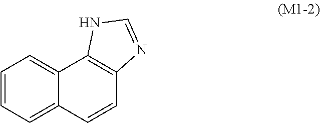 [SMILES: c1ccc2c(c1)ccc1nc[nH]c12]

In the formula (M1-2), the carbon atom in the formula (M1) corresponds to a carbon atom at the 2-position of a 1H-naphtho[1,2-d]imidazole skeleton of the formula (M1-2), and the nitrogen atom in the formula (M1) corresponds to a nitrogen atom at the 3-position of the 1H-naphtho[1,2-d]imidazole skeleton of the formula (M1-2). That is, the two wavy lines terminating the two bonds extending from the carbon atom in the formula (M1) represent a hydrogen atom (omitted in the formula (M1-2)) bonded to the carbon atom in the formula (M1-2) and a nitrogen atom at the 1-position of the 1H-naphtho[1,2-d]imidazole skeleton in the formula (M1-2), and the wavy line terminating the bond extending from the nitrogen atom in the formula (M1) represents a carbon atom at the 4-position of the 1H-naphtho[1,2-d]imidazole skeleton in the formula (M1-2).

Therefore, the first component in the first organic EL device according to an aspect of the present invention does not have an imidazole skeleton.

Here, as understood from the formula (M1), each of atoms is present beyond each of the bond extending from the nitrogen atom and the bonds extending from the carbon atom, and it is excluded that the bond extending from the nitrogen atom and the bond extending from the carbon atom are one single bond (identical). In other words, an atom present beyond the wavy line terminating the bond extending from the nitrogen atom cannot be the carbon atom bonded to the nitrogen atom via a double bond, and an atom present beyond the wavy line terminating the bond extending from the carbon atom cannot be the nitrogen atom bonded to the carbon atom via a double bond. That is, a cyano group in which a nitrogen atom and a carbon atom are bonded via a triple bond does not correspond to the structure represented by the formula (M1). As understood from the definition, the cyano group corresponds to the structure represented by the formula (M3).

The structure represented by the formula (M2) is derived from a phosphine oxide group, that is, the first component in the first organic EL device according to an aspect of the present invention does not have a phosphine oxide group.

Here, as understood from the formula (M2), it is excluded that two or more wavy lines terminating a bond extending from a phosphorus atom mean the presence of one common atom beyond the two or more wavy lines. For example, a case where a phosphorus atom is bonded to one carbon atom via a double bond is not included. That is, since a group represented by the following (M2-1) (a monovalent group derived from methylidene phosphoryl) does not correspond to the structure represented by the formula (M2), the first component in the first organic EL device according to an aspect of the present invention may have a structure represented by the formula (M2-1):

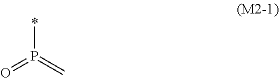 [SMILES: *P(=C)=O]

wherein in the formula (M2-1), * represents a bonding position of the substituent.

The structure represented by the formula (M3) is derived from a cyano group, that is, the first component in the first organic EL device according to an aspect of the present invention does not have a cyano group.

Here, as understood from the formula (M3), a wavy line terminating a bond extending from a carbon atom means that one atom is present beyond the wavy line.

The structure represented by the formula (M4) is derived from a carbonyl group, that is, the first component in the first organic EL device according to an aspect of the present invention does not have a carbonyl group.

Here, as understood from the formula (M4), it is excluded that two wavy lines terminating a bond extending from one carbon atom mean the presence of one common atom beyond the two wavy lines. For example, a case where one carbon atom is bonded to one carbon atom via a double bond is not included. That is, since a group represented by the following (M4-1) (a monovalent group derived from ethenone) does not correspond to the structure represented by the formula (M4), the first component in the first organic EL device according to an aspect of the present invention may have a structure represented by the formula (M4-1):

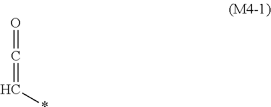 [SMILES: *C=C=O]

wherein in the formula (M4-1), * represents a bonding position of the substituent.

As understood from the definition, the substituent A and R₉₀₁ to R₉₀₇ being the “substituted or unsubstituted monovalent heterocyclic group having 5 to 50 ring atoms” in the formula (1) do not have the structures represented by the formulas (M1) to (M4).

In the formula (1), L_{1A} being the “substituted or unsubstituted divalent heterocyclic group having 5 to 50 ring atoms” does not have the structures represented by the formulas (M1) to (M4). In the formula (1A), when “one or more sets of the adjacent two or more of R_{11A} to R_{18A} form
a substituted or unsubstituted single ring by bonding with each other, or form a substituted or unsubstituted fused ring by bonding with each other”, the single ring or the fused ring does not have the structures represented by the formulas (M1) to (M4).

In the formula (1) and the formula (1A), the substituent in the case of “substituted or unsubstituted” does not have the structures represented by the formulas (M1) to (M4).

In one embodiment, at least three of R₁ to R₁₀ are independently the group represented by the formula (1A), or the substituent A.

In one embodiment, two of R₁ to R₁₀ are independently the substituent A, and one of the other R₁ to R₁₀ is the group represented by the formula (1A).

In one embodiment, R₉ and R₁₀ are independently the group represented by the formula (1A), or the substituent A.

In one embodiment, one of R₉ and R₁₀ is the group represented by the formula (1A), and the other of R₉ and R₁₀ is the substituent A.

In one embodiment, R₂, R₉, and R₁₀ are independently the group represented by the formula (1A), or the substituent A.

In one embodiment, one of R₉ and R₁₀ is the group represented by the formula (1A), and the other of R₉ and R₁₀ and R₂ are independently the substituent A.

In one embodiment, the group represented by the formula (1A) is a group represented by any one of the following formulas (1A-1) to (1A-3):

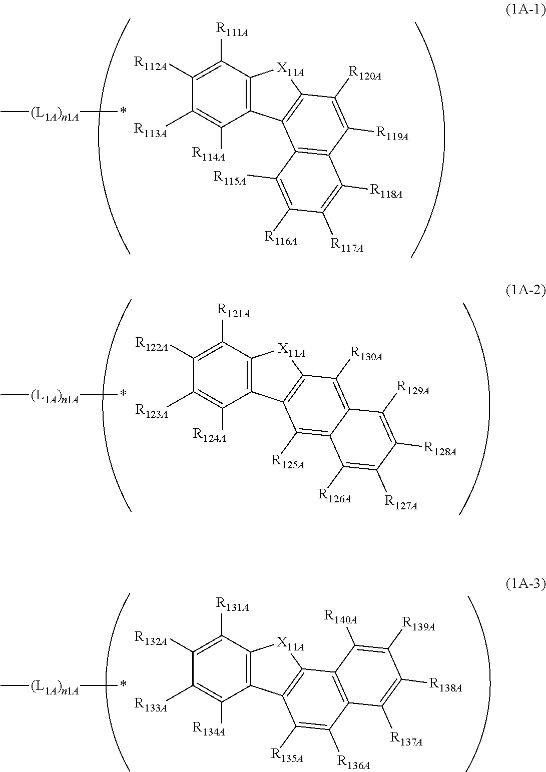

wherein in the formulas (1A-1) to (1A-3), L_{1A} and n1A are as defined in the formula (1);
X_{11A} is C(R_{21A})(R_{22A}), N(R_{23A}), O, or S;
one of R_{21A} to R_{23A}, R_{111A} to R_{120A}, R_{121A} to R_{130A}, and R_{131A} to R_{140A} represents a bond to L_{1A};
R_{21A} to R_{23A}, R_{111A} to R_{120A}, R_{121A} to R_{130A}, and R_{131A} to R_{140A} that do not represent the bond to L_{1A} are independently a hydrogen atom, or a substituent A; and the substituent A is as defined in the formula (1).

In one embodiment, X_{11A} is O or S.

In one embodiment, the compound represented by the formula (1) is a compound represented by any one of the following formulas (1-1) to (1-3):

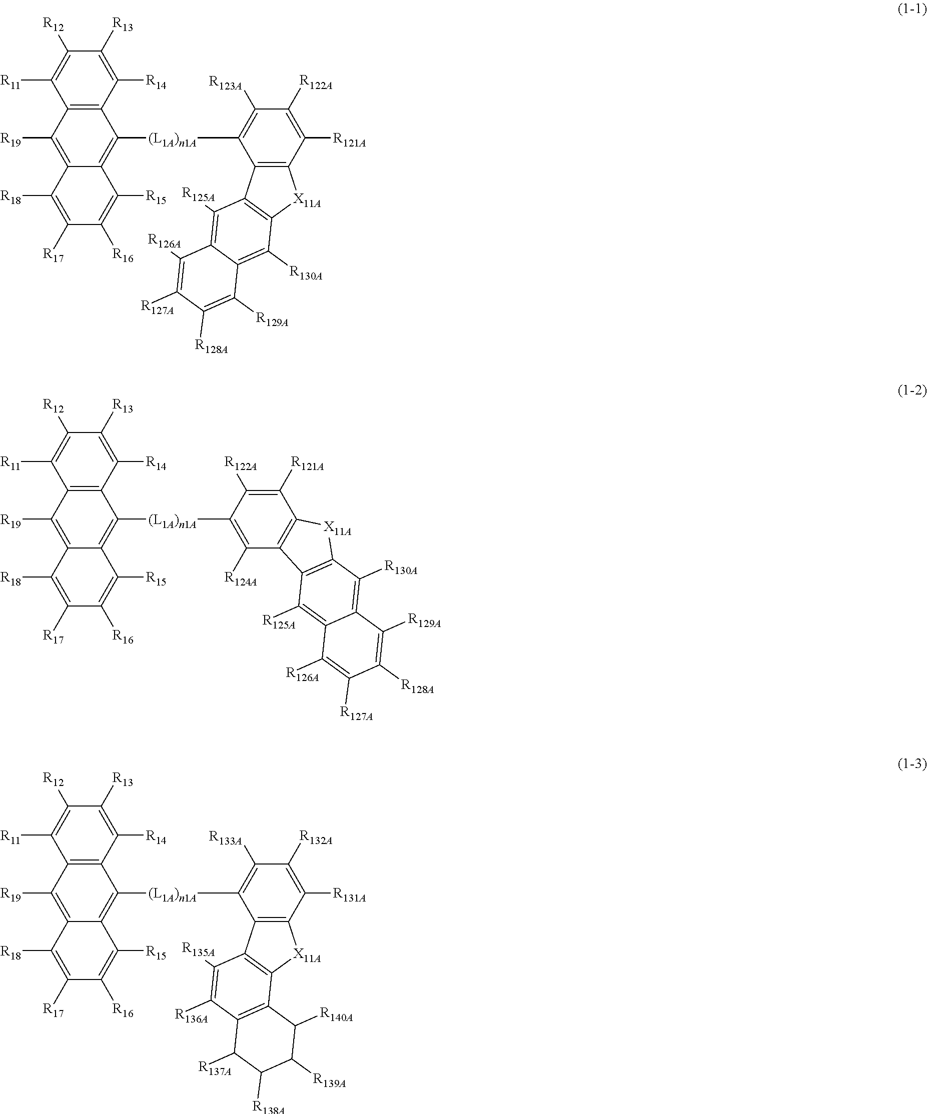

wherein in the formulas (1-1) to (1-3), L_{1A} and n1A are as defined in the formula (1);
X_{111A} is 0 or S;
R₁₂ and R₁₉ are independently a substituent A;
R₁₁, R₁₃ to R₁₈, and R_{121A} to R_{140A} are independently a hydrogen atom or a substituent A; and
the substituent A is as defined in the formula (1),
provided that the compound represented by any one of the formulas (1-1) to (1-3) does not have a structure represented by the following formula (M1), a structure represented by the following formula (M2), a structure represented by the following formula (M3), and a structure represented by the following formula (M4) in the molecule thereof:

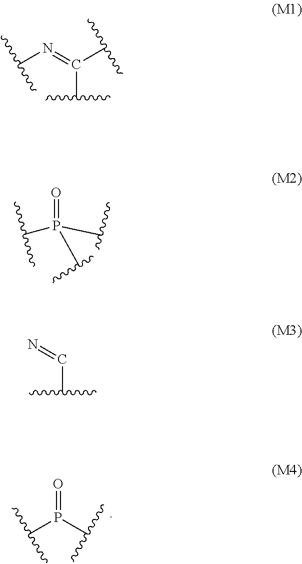

In one embodiment, L_{1A} is a single bond.

In one embodiment, the compound represented by the formula (1) is a compound represented by any one of the following formulas (1-11) to (1-41):

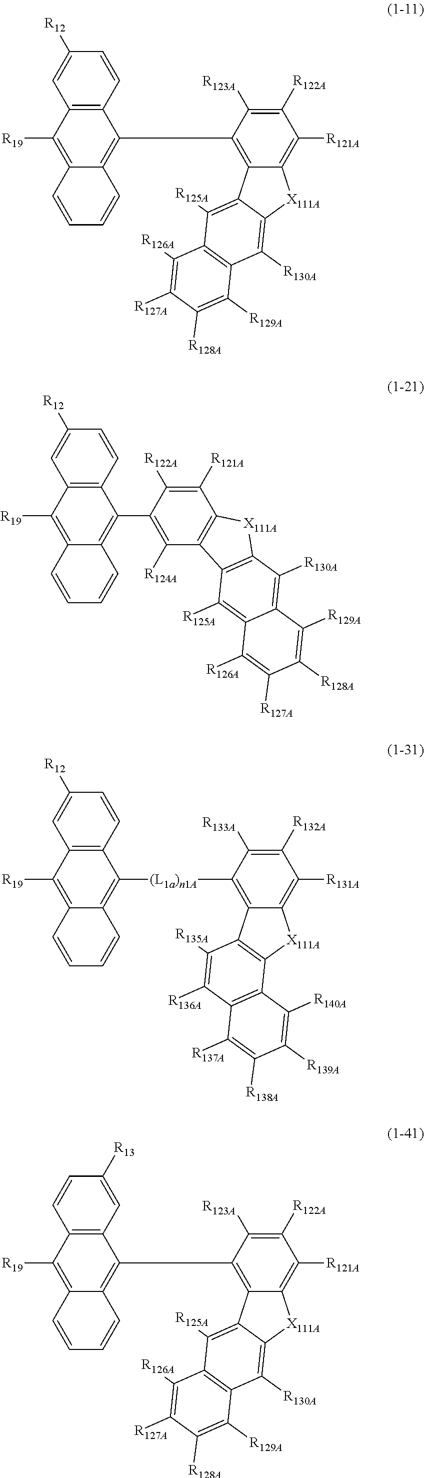

wherein in the formulas (1-11) to (1-41),
X_{111A} is O or S;
R₁₂, R₁₃, and R₁₉ are independently a substituent A;
R_{121A} to R_{140A} are independently a hydrogen atom or a substituent A; and
the substituent A is as defined in the formula (1),
provided that the compound represented by any one of the formulas (1-11) to (1-41) does not have a structure represented by the following formula (M1), a structure represented by the following formula (M2), a structure represented by the following formula (M3), and a structure represented by the following formula (M4) in the molecule thereof:

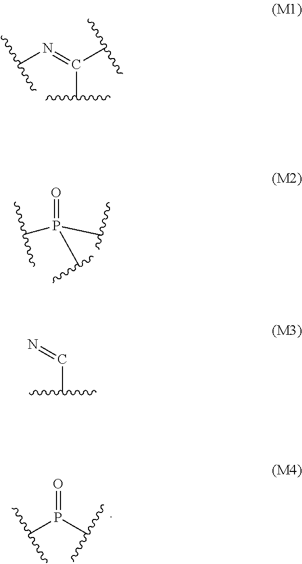

In one embodiment, a substituent in the case of “substituted or unsubstituted” in the formula (1) is selected from the group consisting of
an alkyl group having 1 to 50 carbon atoms,
a haloalkyl group having 1 to 50 carbon atoms,
an alkenyl group having 2 to 50 carbon atoms,
an alkynyl group having 2 to 50 carbon atoms,
a cycloalkyl group having 3 to 50 ring carbon atoms,
an alkoxy group having 1 to 50 carbon atoms,
an alkylthio group having 1 to 50 carbon atoms,
an aryloxy group having 6 to 50 ring carbon atoms,
an arylthio group having 6 to 50 ring carbon atoms,
an aralkyl group having 7 to 50 carbon atoms,

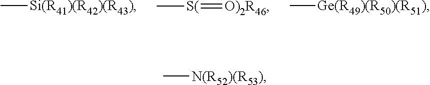

a hydroxy group,
a halogen atom,
a nitro group,
an aryl group having 6 to 50 ring carbon atoms, and
a monovalent heterocyclic group having 5 to 50 ring atoms.
R₄₁ to R₄₃, R₄₆, and R₄₉ to R₅₃ are independently a hydrogen atom, an alkyl group having 1 to 50 carbon atoms, an aryl group having 6 to 50 ring carbon atoms, or a monovalent heterocyclic group having 5 to 50 ring atoms. When two or more of each of R₄₁ to R₄₃, R₄₆, and R₄₉ to R₅₃ are present, the two or more of each of R₄₁ to R₄₃, R₄₆, and R₄₉ to R₅₃ may be the same as or different from each other.

In one embodiment, a substituent in the case of “substituted or unsubstituted” in the above formula (1) is selected from the group consisting of
an alkyl group having 1 to 50 carbon atoms,
an aryl group having 6 to 50 ring carbon atoms, and
a monovalent heterocyclic group having 5 to 50 ring atoms.

In one embodiment, a substituent in the case of “substituted or unsubstituted” in the above formula (1) is selected from the group consisting of
an alkyl group having 1 to 18 carbon atoms,
an aryl group having 6 to 18 ring carbon atoms, and
a monovalent heterocyclic group having 5 to 18 ring atoms.

In the present specification, the expression “hydrogen atoms are a deuterium atom” means that the amount of the deuterium atom based on the total amount of the protium atom and the deuterium atom in the hydrogen atom is larger than the natural abundance ratio. It can be confirmed by using a nuclear magnetic resonance apparatus that the amount of the deuterium atom based on the total amount of the protium atom and the deuterium atom is higher than the natural abundance ratio.

The compound represented by the formula (1) can be synthesized by using a known reaction or raw material suited for an intended product.

Specific examples of the compound represented by the formula (1) will be described hereinafter, but these are merely examples, and a compound according to an aspect of the present invention is not limited to the following specific examples.

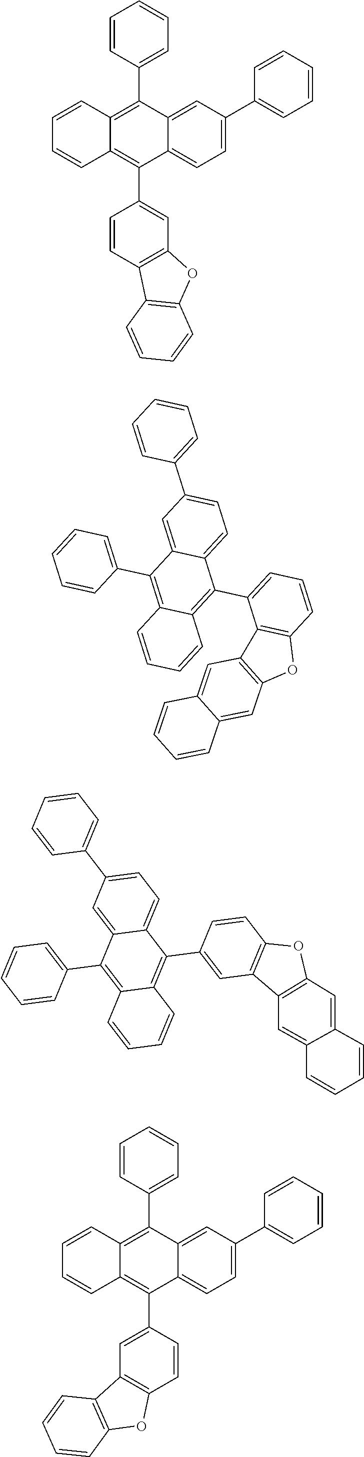

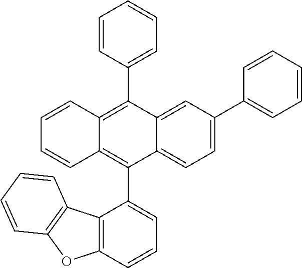 [SMILES: c1ccc(-c2ccc3c(-c4cccc5oc6ccccc6c45)c4ccccc4c(-c4ccccc4)c3c2)cc1]

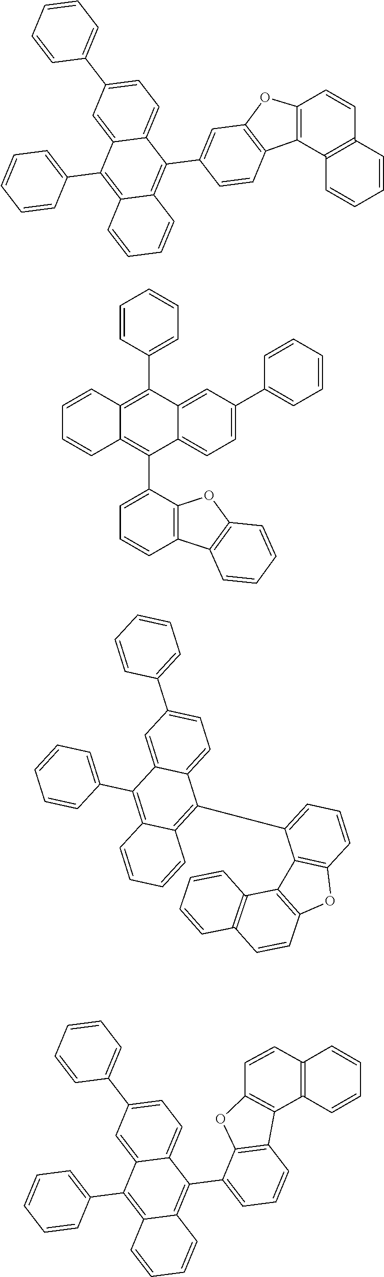

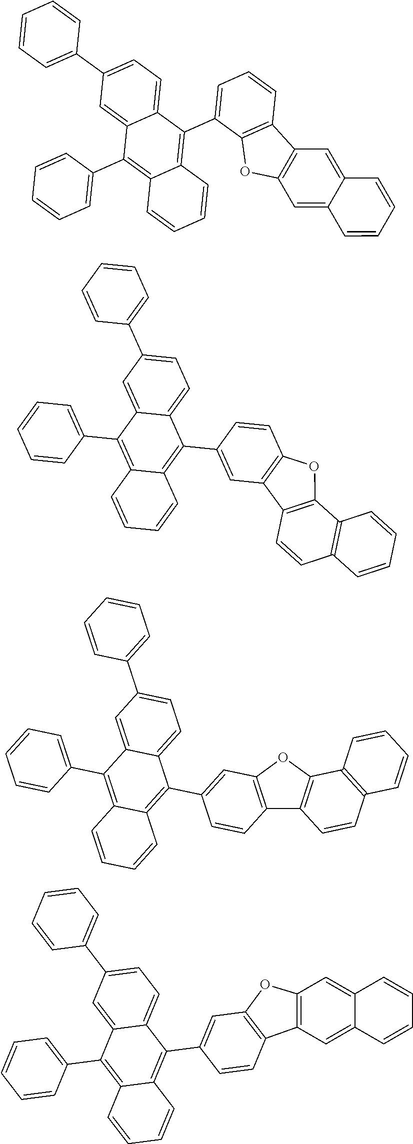

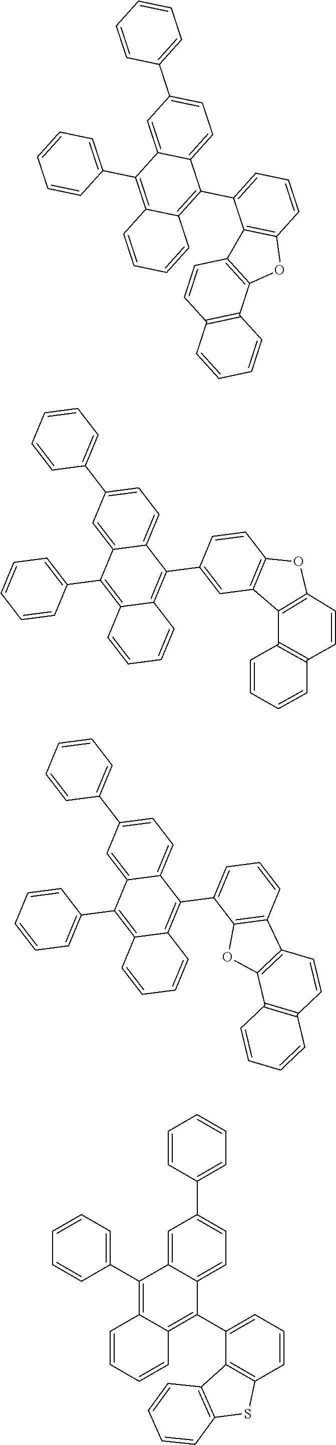 [SMILES: c1ccc(-c2ccc3c(-c4cccc5oc6c7ccccc7ccc6c45)c4ccccc4c(-c4ccccc4)c3c2)cc1 ; c1ccc(-c2ccc3c(-c4ccc5oc6ccc7ccccc7c6c5c4)c4ccccc4c(-c4ccccc4)c3c2)cc1 ; c1ccc(-c2ccc3c(-c4cccc5c4oc4c6ccccc6ccc54)c4ccccc4c(-c4ccccc4)c3c2)cc1 ; c1ccc(-c2ccc3c(-c4cccc5sc6ccccc6c45)c4ccccc4c(-c4ccccc4)c3c2)cc1]

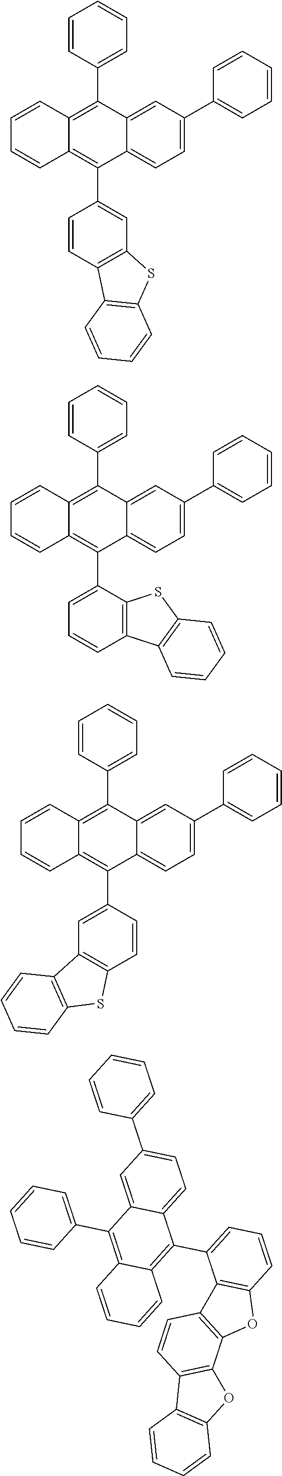

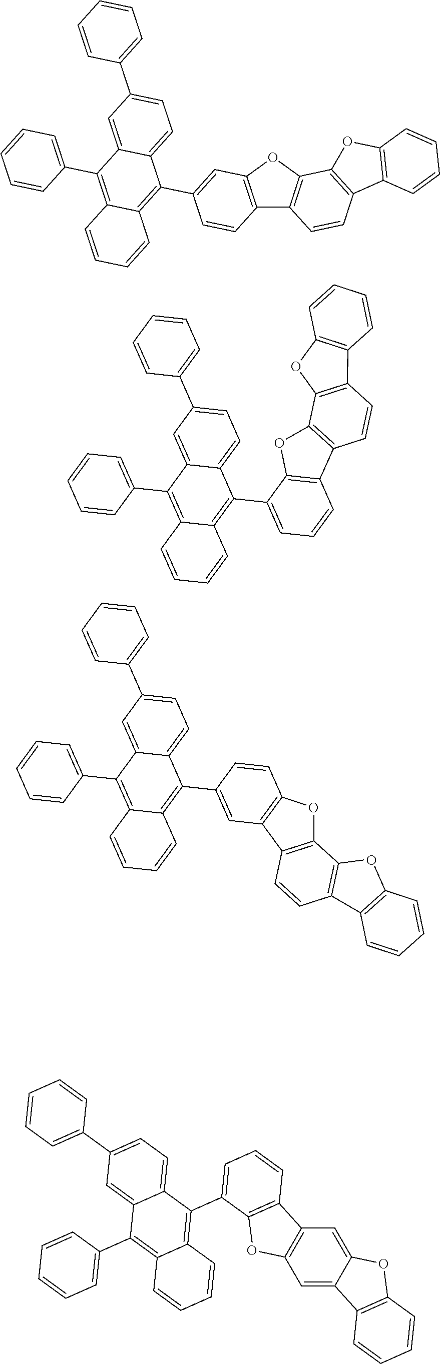 [SMILES: c1ccc(-c2ccc3c(-c4ccc5c(c4)oc4c5ccc5c6ccccc6oc54)c4ccccc4c(-c4ccccc4)c3c2)cc1 ; c1ccc(-c2ccc3c(-c4cccc5c4oc4c5ccc5c6ccccc6oc54)c4ccccc4c(-c4ccccc4)c3c2)cc1 ; c1ccc(-c2ccc3c(-c4ccc5oc6c(ccc7c8ccccc8oc76)c5c4)c4ccccc4c(-c4ccccc4)c3c2)cc1 ; c1ccc(-c2ccc3c(-c4cccc5c4oc4cc6c(cc45)oc4ccccc46)c4ccccc4c(-c4ccccc4)c3c2)cc1]

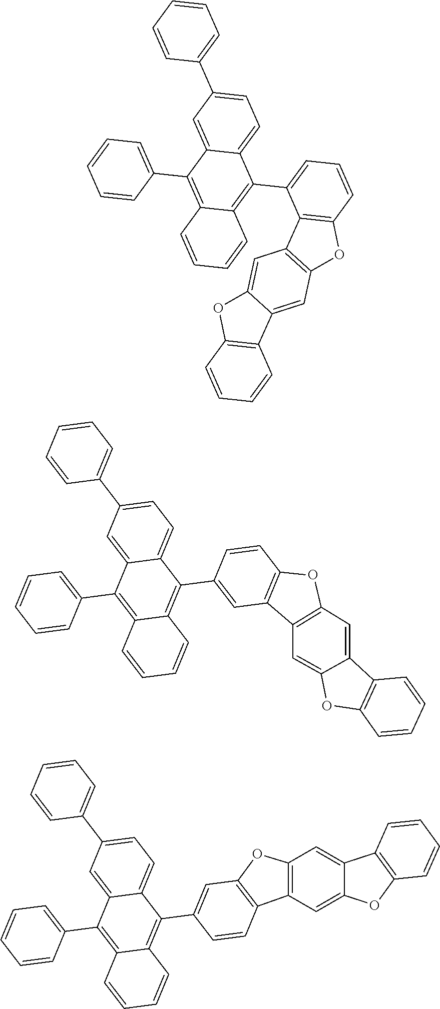 [SMILES: c1ccc(-c2ccc3c(-c4cccc5oc6cc7c(cc6c45)oc4ccccc47)c4ccccc4c(-c4ccccc4)c3c2)cc1 ; c1ccc(-c2ccc3c(-c4ccc5oc6cc7c(cc6c5c4)oc4ccccc47)c4ccccc4c(-c4ccccc4)c3c2)cc1 ; c1ccc(-c2ccc3c(-c4ccc5c(c4)oc4cc6c(cc45)oc4ccccc46)c4ccccc4c(-c4ccccc4)c3c2)cc1]

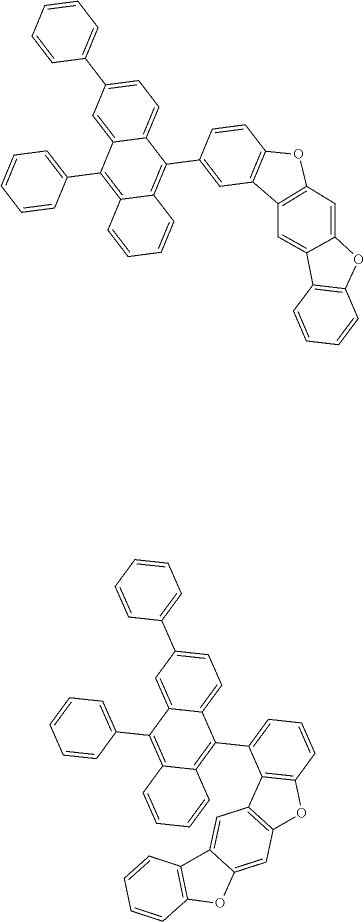 [SMILES: c1ccc(-c2ccc3c(-c4ccc5oc6cc7oc8ccccc8c7cc6c5c4)c4ccccc4c(-c4ccccc4)c3c2)cc1 ; c1ccc(-c2ccc3c(-c4cccc5oc6cc7oc8ccccc8c7cc6c45)c4ccccc4c(-c4ccccc4)c3c2)cc1]

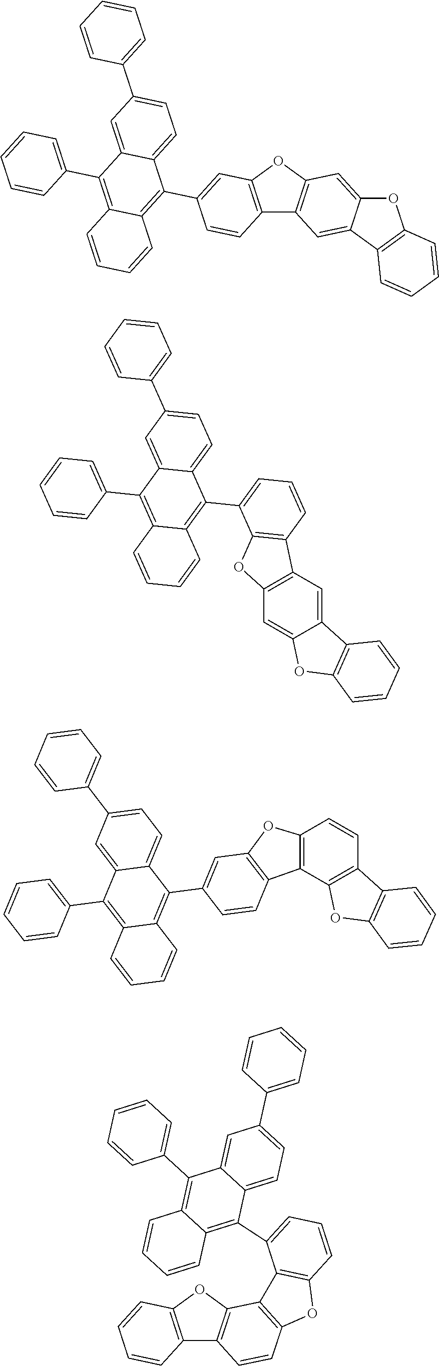 [SMILES: c1ccc(-c2ccc3c(-c4ccc5c(c4)oc4cc6oc7ccccc7c6cc45)c4ccccc4c(-c4ccccc4)c3c2)cc1 ; c1ccc(-c2ccc3c(-c4cccc5c4oc4cc6oc7ccccc7c6cc45)c4ccccc4c(-c4ccccc4)c3c2)cc1 ; c1ccc(-c2ccc3c(-c4ccc5c(c4)oc4ccc6c7ccccc7oc6c45)c4ccccc4c(-c4ccccc4)c3c2)cc1 ; c1ccc(-c2ccc3c(-c4cccc5oc6ccc7c8ccccc8oc7c6c45)c4ccccc4c(-c4ccccc4)c3c2)cc1]

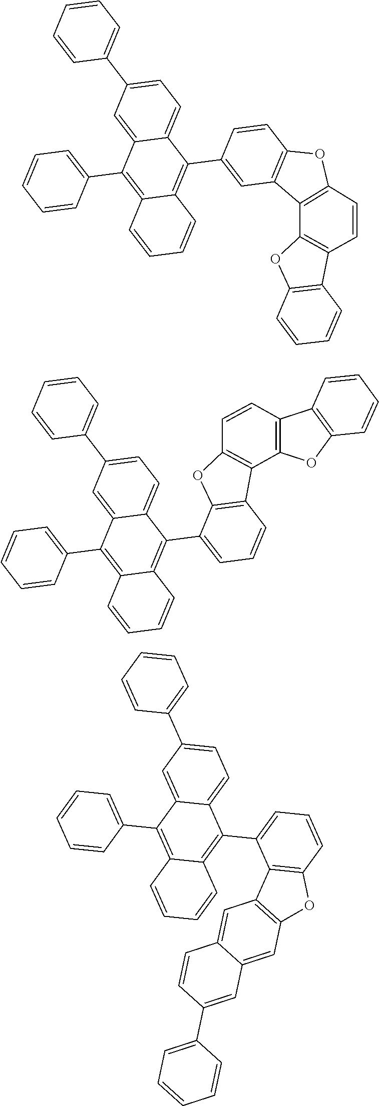

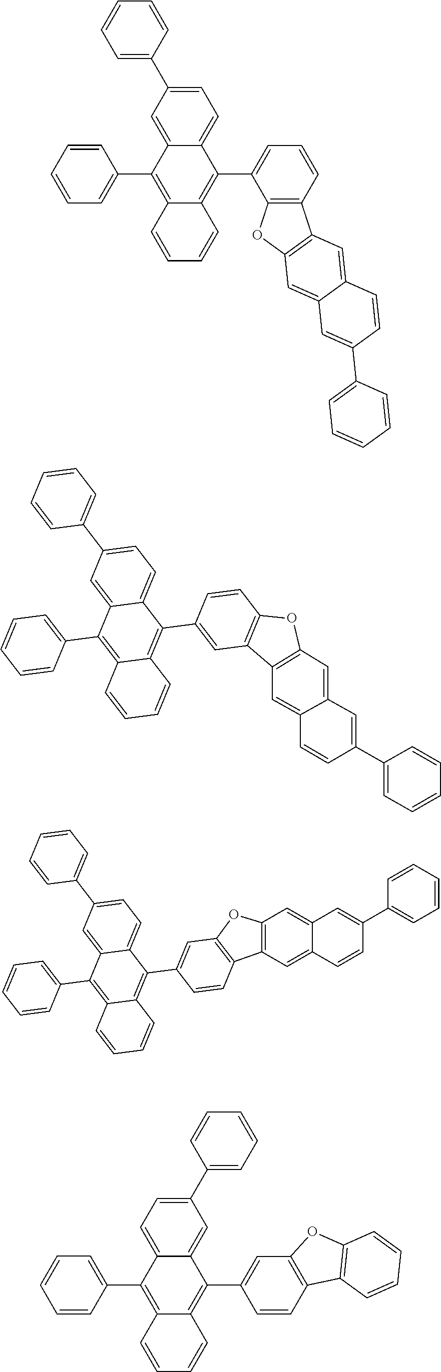 [SMILES: c1ccc(-c2ccc3cc4c(cc3c2)oc2c(-c3c5ccccc5c(-c5ccccc5)c5cc(-c6ccccc6)ccc35)cccc24)cc1 ; c1ccc(-c2ccc3cc4c(cc3c2)oc2ccc(-c3c5ccccc5c(-c5ccccc5)c5cc(-c6ccccc6)ccc35)cc24)cc1 ; c1ccc(-c2ccc3cc4c(cc3c2)oc2cc(-c3c5ccccc5c(-c5ccccc5)c5cc(-c6ccccc6)ccc35)ccc24)cc1 ; c1ccc(-c2ccc3c(-c4ccccc4)c4ccccc4c(-c4ccc5c(c4)oc4ccccc45)c3c2)cc1]

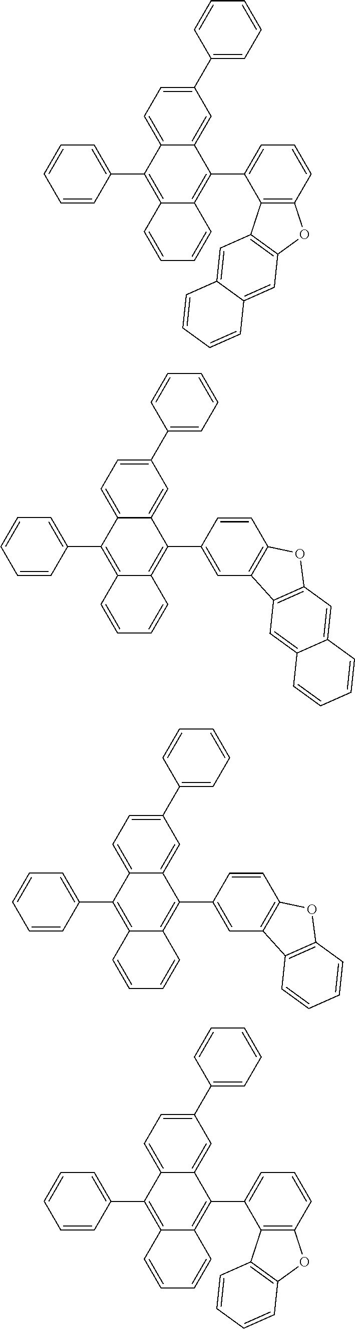

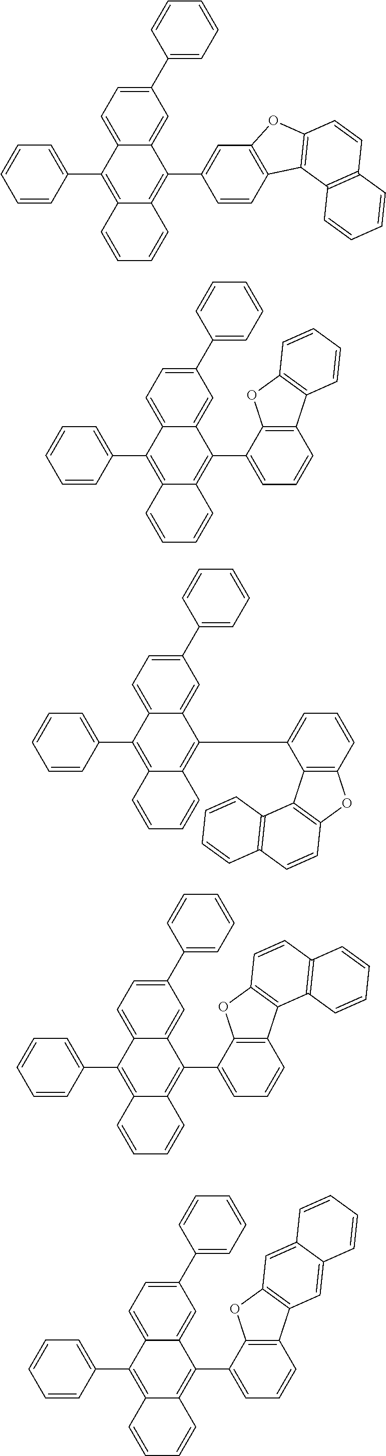

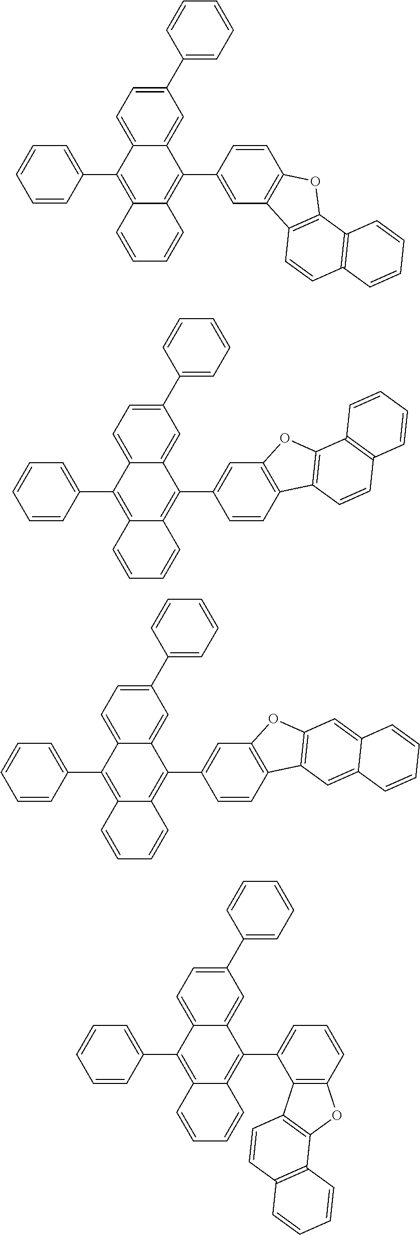

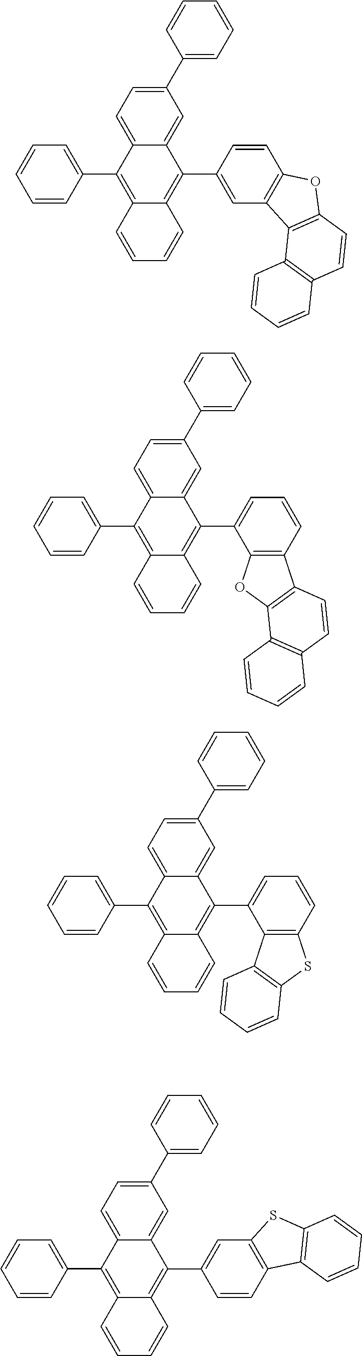

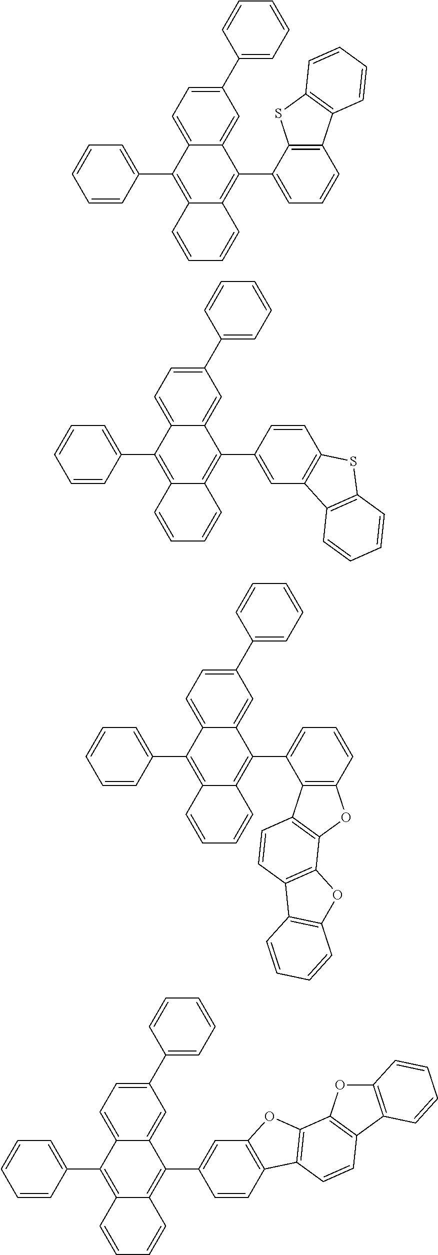

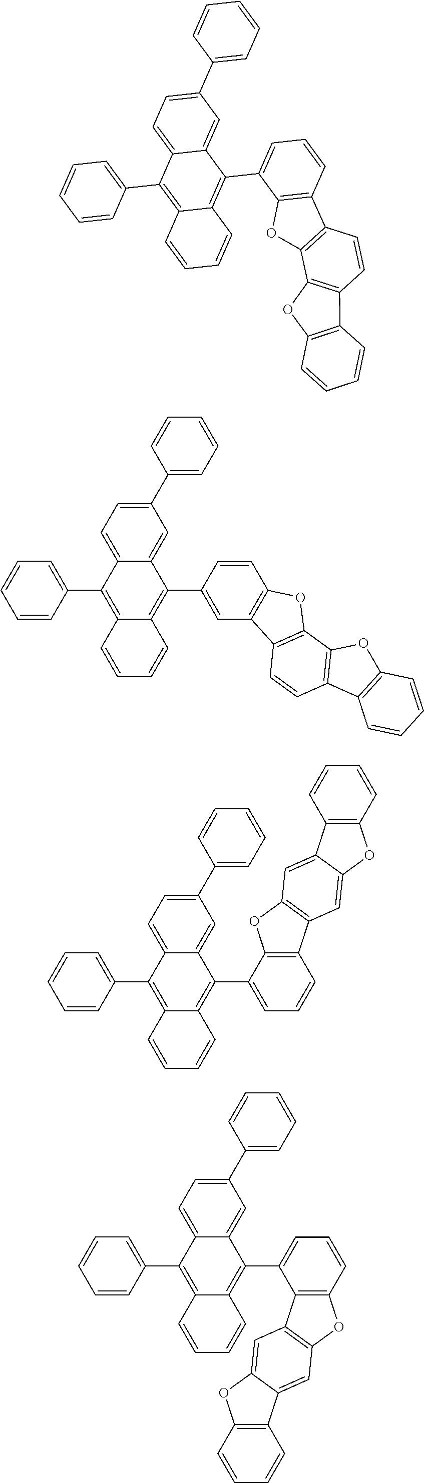

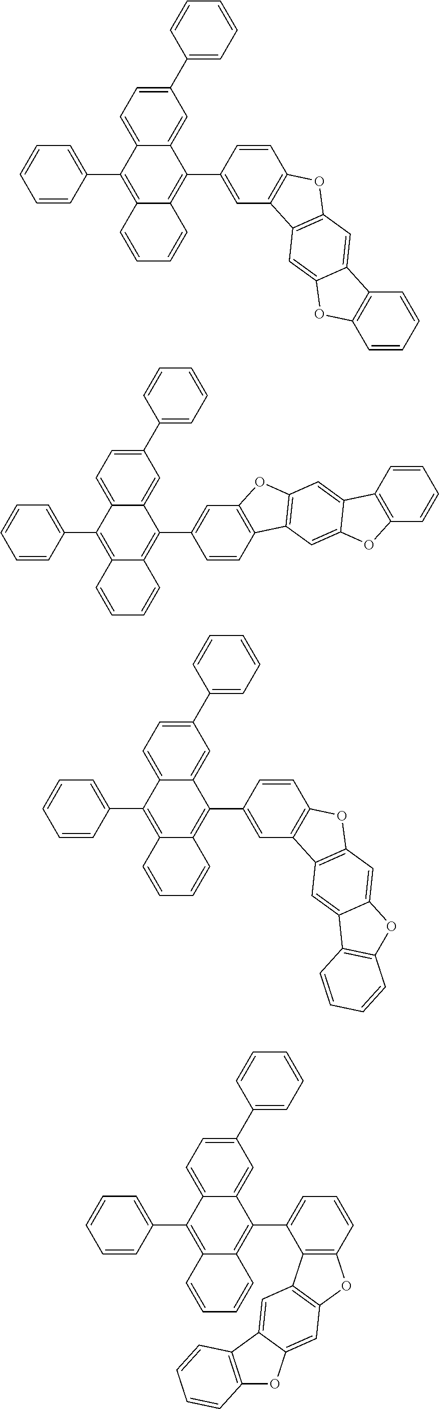 [SMILES: c1ccc(-c2ccc3c(-c4ccccc4)c4ccccc4c(-c4ccc5oc6cc7c(cc6c5c4)oc4ccccc47)c3c2)cc1 ; c1ccc(-c2ccc3c(-c4ccccc4)c4ccccc4c(-c4ccc5c(c4)oc4cc6c(cc45)oc4ccccc46)c3c2)cc1 ; c1ccc(-c2ccc3c(-c4ccccc4)c4ccccc4c(-c4ccc5oc6cc7oc8ccccc8c7cc6c5c4)c3c2)cc1 ; c1ccc(-c2ccc3c(-c4ccccc4)c4ccccc4c(-c4cccc5oc6cc7oc8ccccc8c7cc6c45)c3c2)cc1]

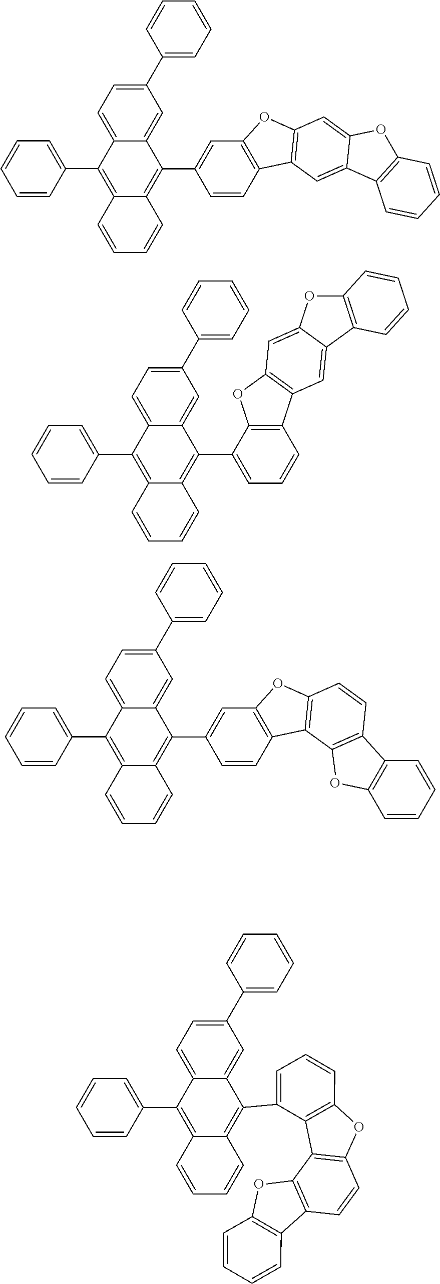

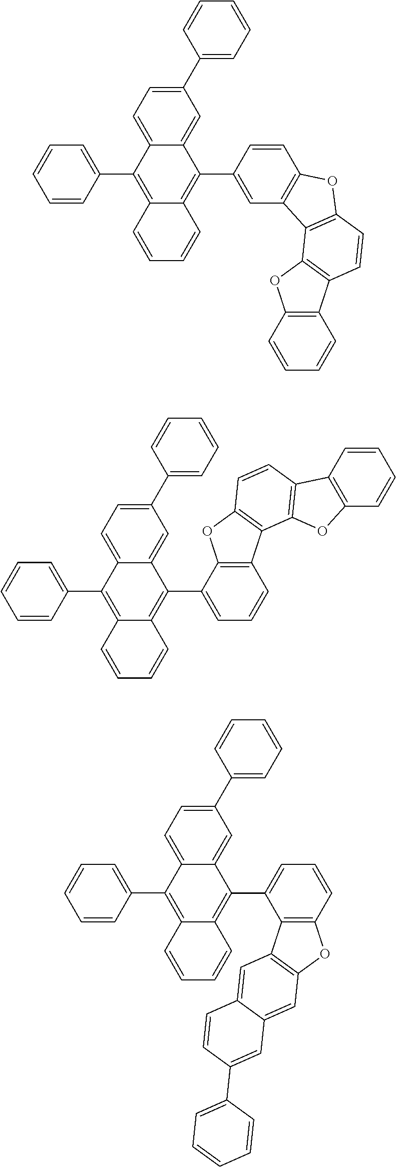 [SMILES: c1ccc(-c2ccc3c(-c4ccccc4)c4ccccc4c(-c4ccc5oc6ccc7c8ccccc8oc7c6c5c4)c3c2)cc1 ; c1ccc(-c2ccc3c(-c4ccccc4)c4ccccc4c(-c4cccc5c4oc4ccc6c7ccccc7oc6c45)c3c2)cc1 ; c1ccc(-c2ccc3cc4c(cc3c2)oc2cccc(-c3c5ccccc5c(-c5ccccc5)c5ccc(-c6ccccc6)cc35)c24)cc1]

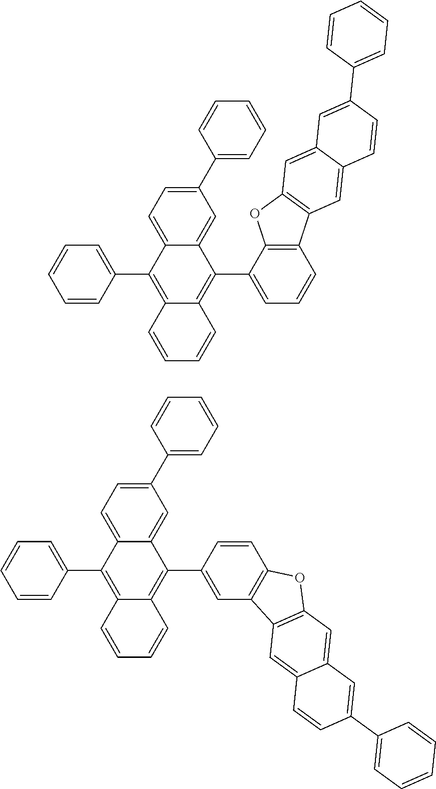

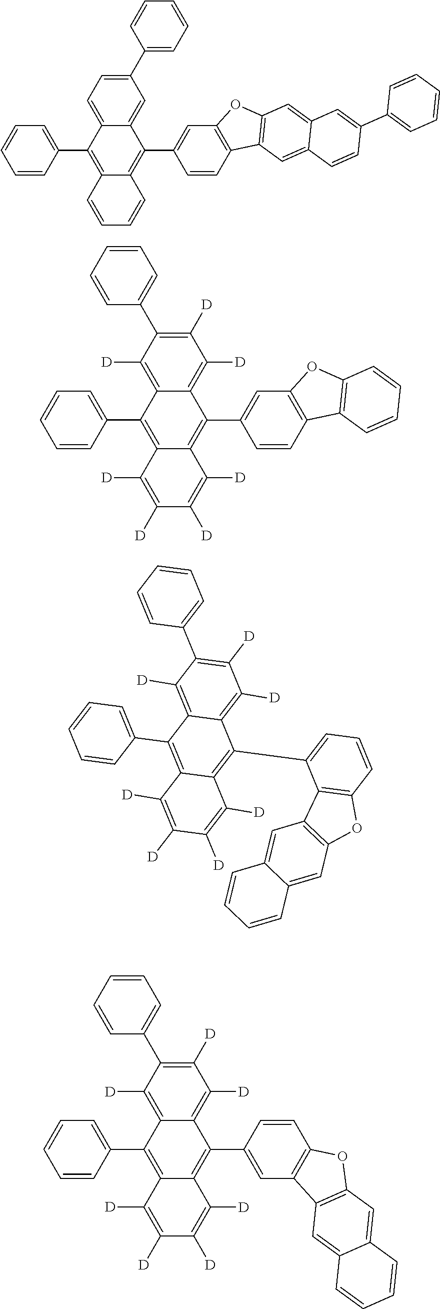

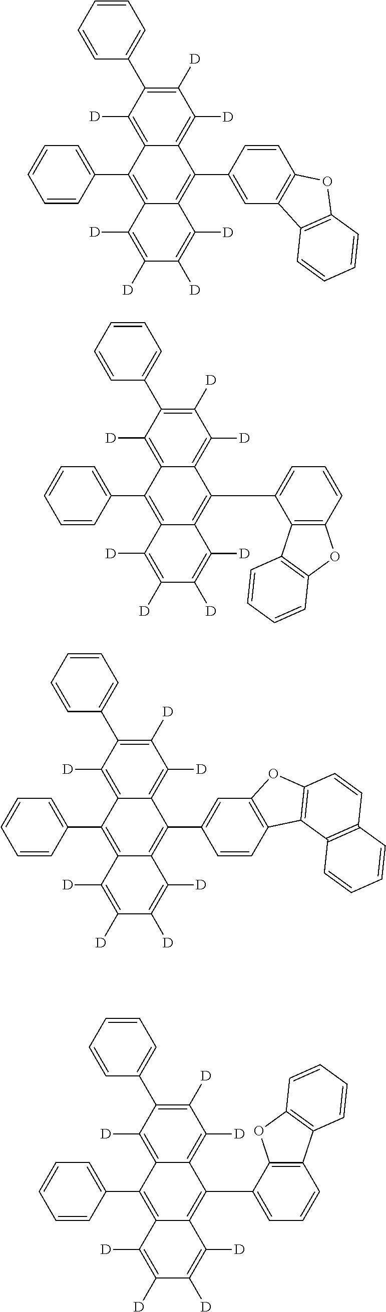

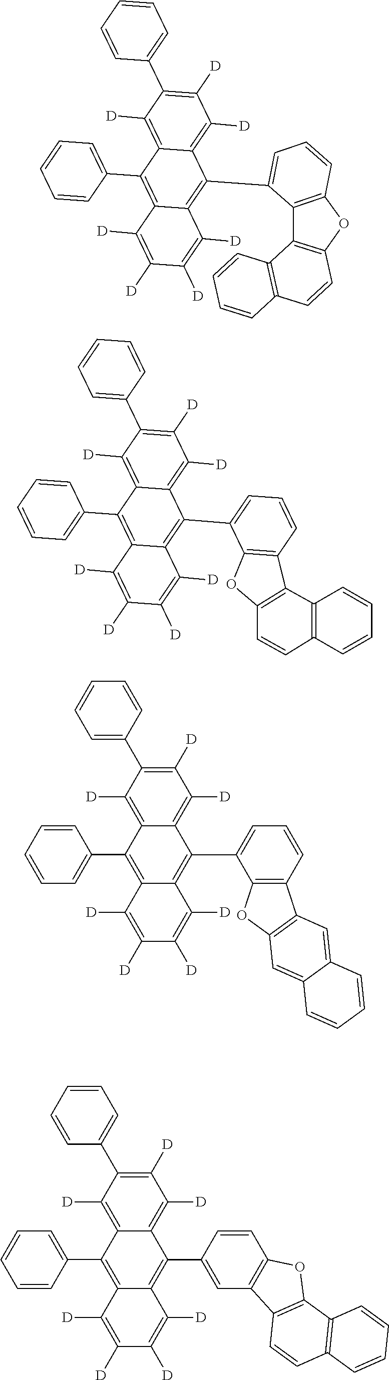 [SMILES: [2H]c1c([2H])c([2H])c2c(-c3cccc4oc5ccc6ccccc6c5c34)c3c([2H])c([2H])c(-c4ccccc4)c([2H])c3c(-c3ccccc3)c2c1[2H] ; [2H]c1c([2H])c([2H])c2c(-c3cccc4c3oc3ccc5ccccc5c34)c3c([2H])c([2H])c(-c4ccccc4)c([2H])c3c(-c3ccccc3)c2c1[2H] ; [2H]c1c([2H])c([2H])c2c(-c3cccc4c3oc3cc5ccccc5cc34)c3c([2H])c([2H])c(-c4ccccc4)c([2H])c3c(-c3ccccc3)c2c1[2H] ; [2H]c1c([2H])c([2H])c2c(-c3ccccc3)c3c([2H])c(-c4ccccc4)c([2H])c([2H])c3c(-c3ccc4oc5c6ccccc6ccc5c4c3)c2c1[2H]]

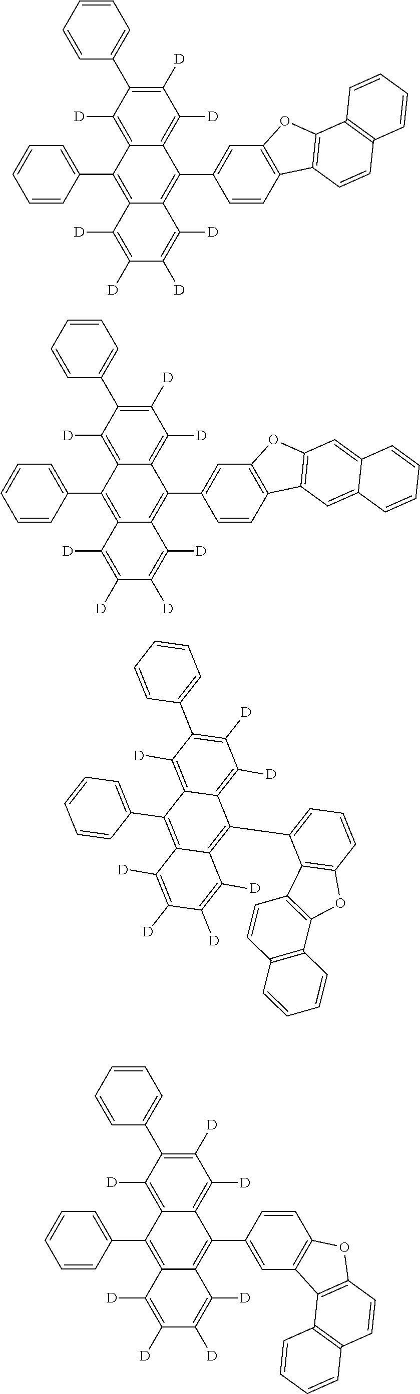

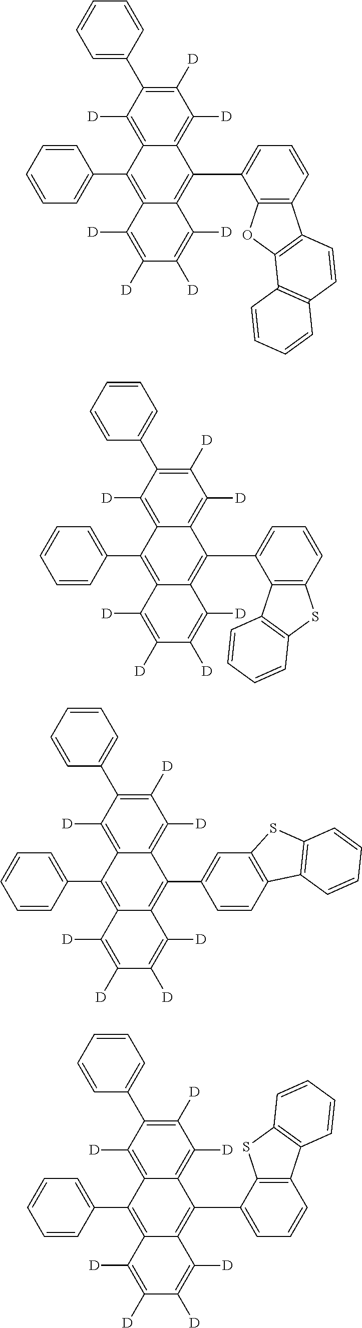

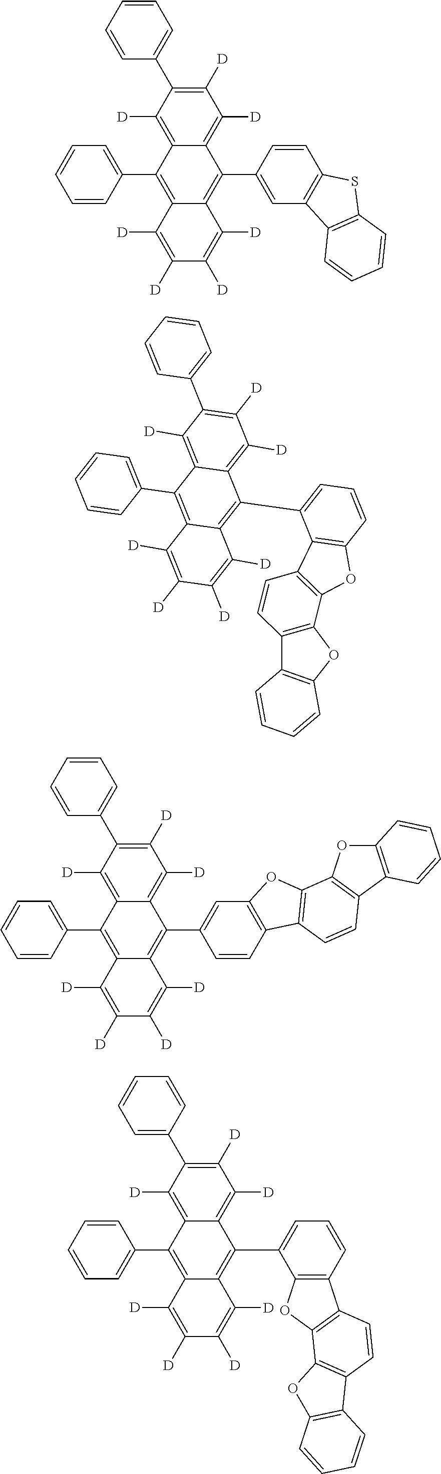

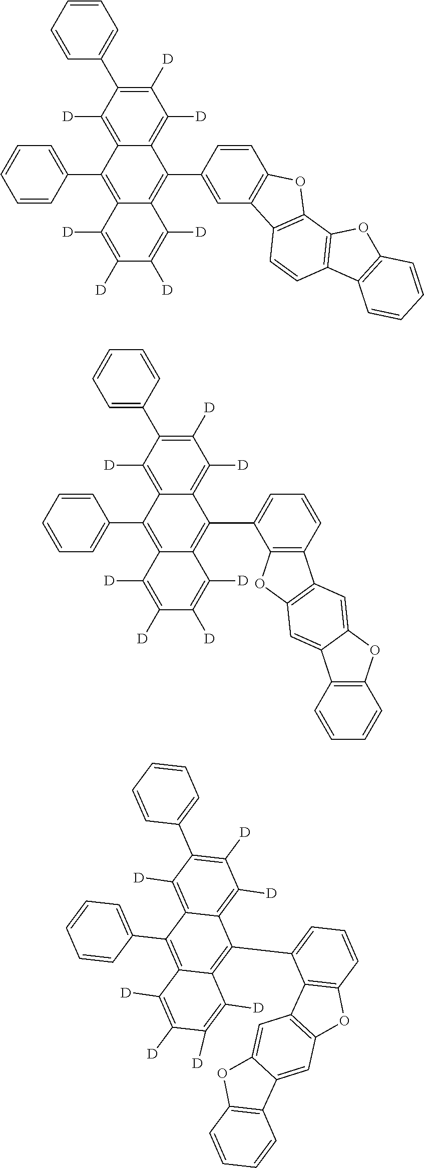 [SMILES: [2H]c1c([2H])c([2H])c2c(-c3ccccc3)c3c([2H])c(-c4ccccc4)c([2H])c([2H])c3c(-c3ccc4oc5c(ccc6c7ccccc7oc65)c4c3)c2c1[2H] ; [2H]c1c([2H])c([2H])c2c(-c3cccc4c3oc3cc5c(cc34)oc3ccccc35)c3c([2H])c([2H])c(-c4ccccc4)c([2H])c3c(-c3ccccc3)c2c1[2H] ; [2H]c1c([2H])c([2H])c2c(-c3cccc4oc5cc6c(cc5c34)oc3ccccc36)c3c([2H])c([2H])c(-c4ccccc4)c([2H])c3c(-c3ccccc3)c2c1[2H]]

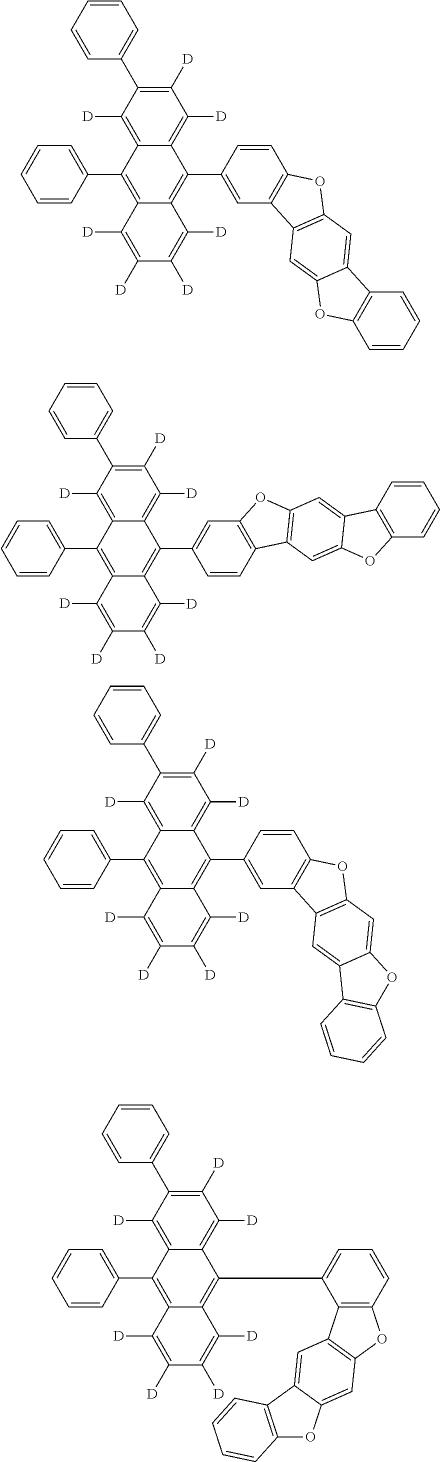

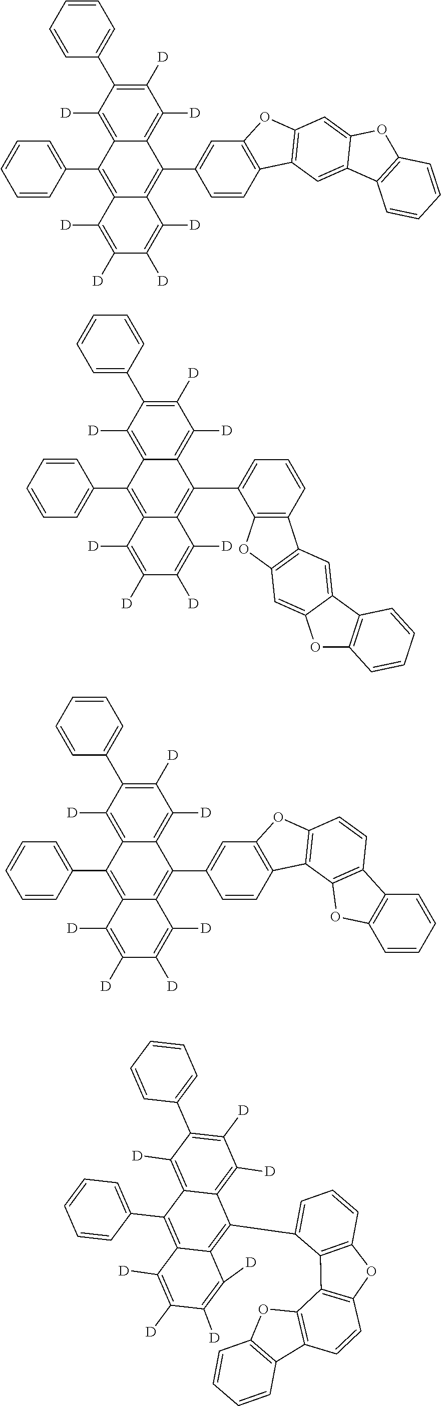

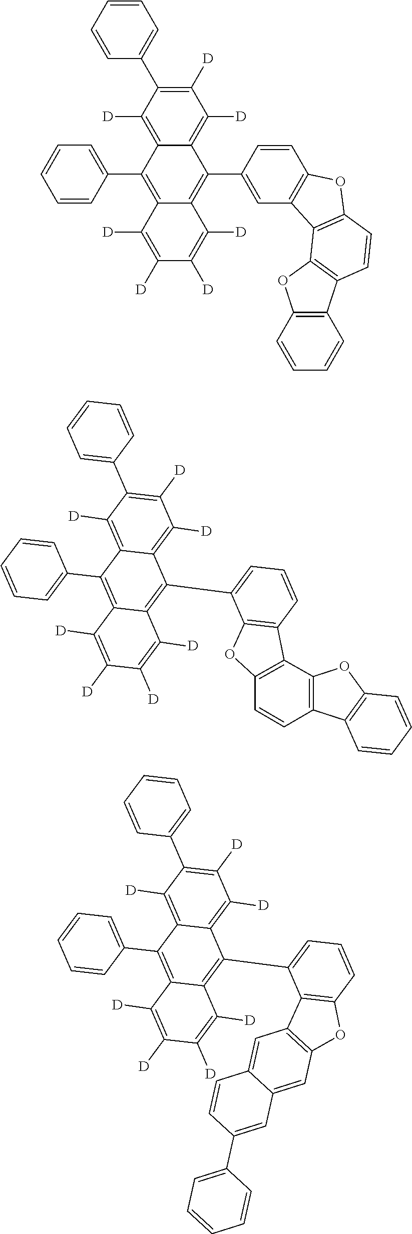 [SMILES: [2H]c1c([2H])c([2H])c2c(-c3ccccc3)c3c([2H])c(-c4ccccc4)c([2H])c([2H])c3c(-c3ccc4oc5ccc6c7ccccc7oc6c5c4c3)c2c1[2H] ; [2H]c1c([2H])c([2H])c2c(-c3cccc4c3oc3ccc5c6ccccc6oc5c34)c3c([2H])c([2H])c(-c4ccccc4)c([2H])c3c(-c3ccccc3)c2c1[2H] ; [2H]c1c([2H])c([2H])c2c(-c3cccc4oc5cc6cc(-c7ccccc7)ccc6cc5c34)c3c([2H])c([2H])c(-c4ccccc4)c([2H])c3c(-c3ccccc3)c2c1[2H]]

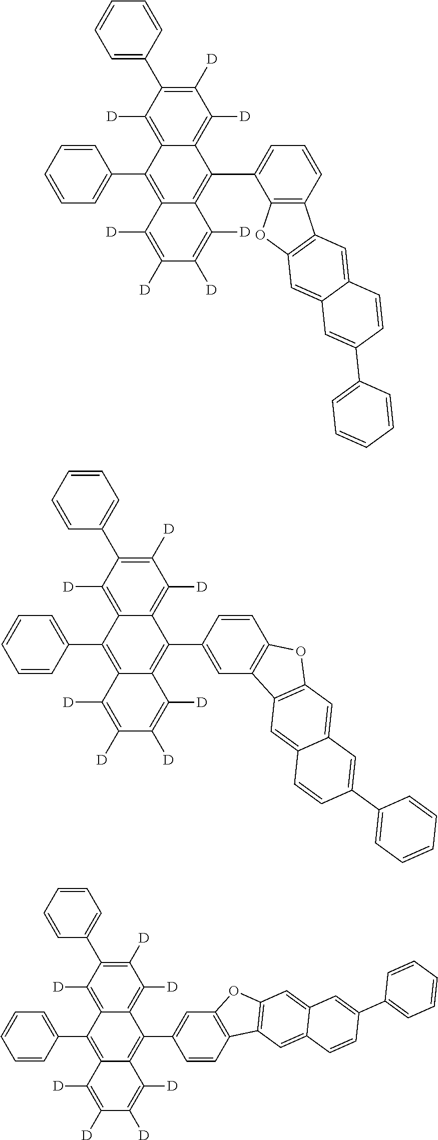 [SMILES: [2H]c1c([2H])c([2H])c2c(-c3cccc4c3oc3cc5cc(-c6ccccc6)ccc5cc34)c3c([2H])c([2H])c(-c4ccccc4)c([2H])c3c(-c3ccccc3)c2c1[2H] ; [2H]c1c([2H])c([2H])c2c(-c3ccccc3)c3c([2H])c(-c4ccccc4)c([2H])c([2H])c3c(-c3ccc4oc5cc6cc(-c7ccccc7)ccc6cc5c4c3)c2c1[2H] ; [2H]c1c([2H])c([2H])c2c(-c3ccccc3)c3c([2H])c(-c4ccccc4)c([2H])c([2H])c3c(-c3ccc4c(c3)oc3cc5cc(-c6ccccc6)ccc5cc34)c2c1[2H]]

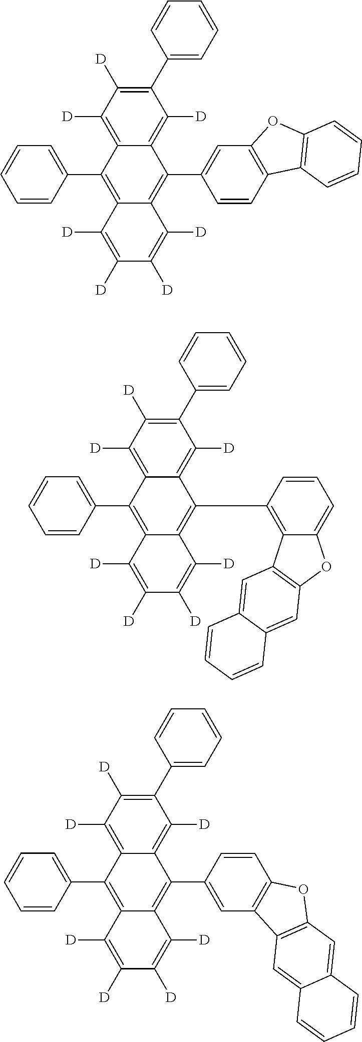

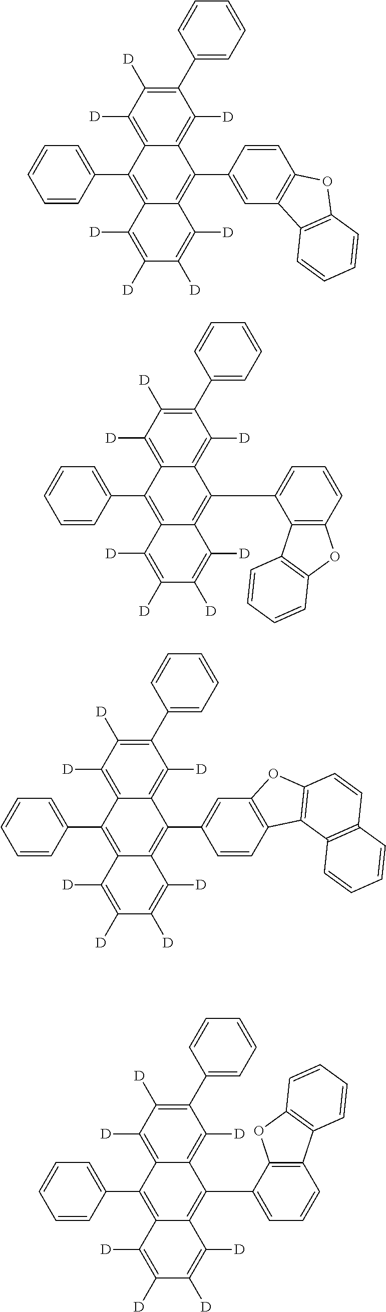

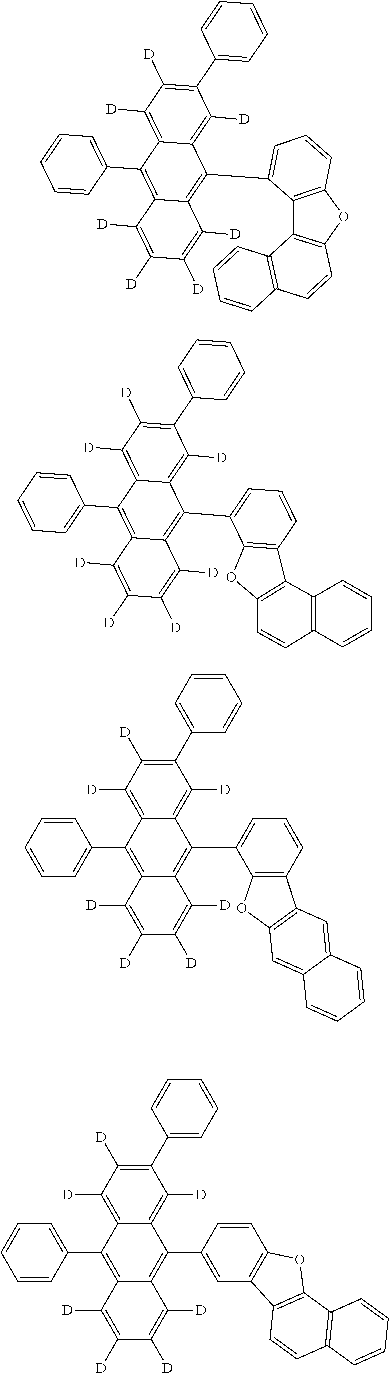

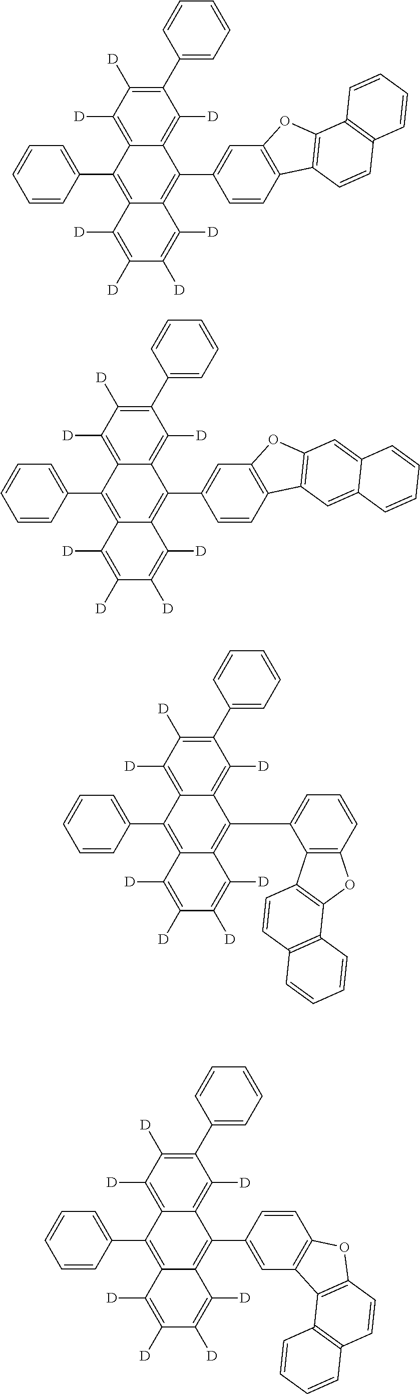

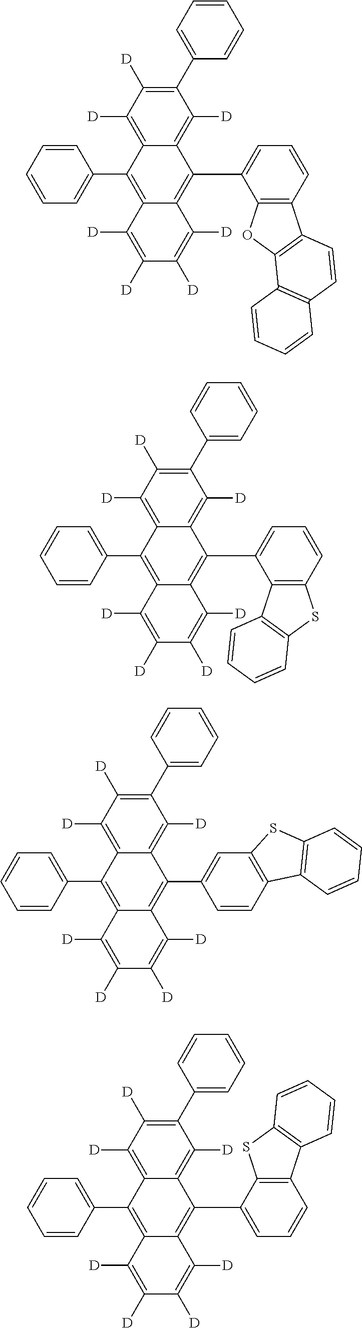

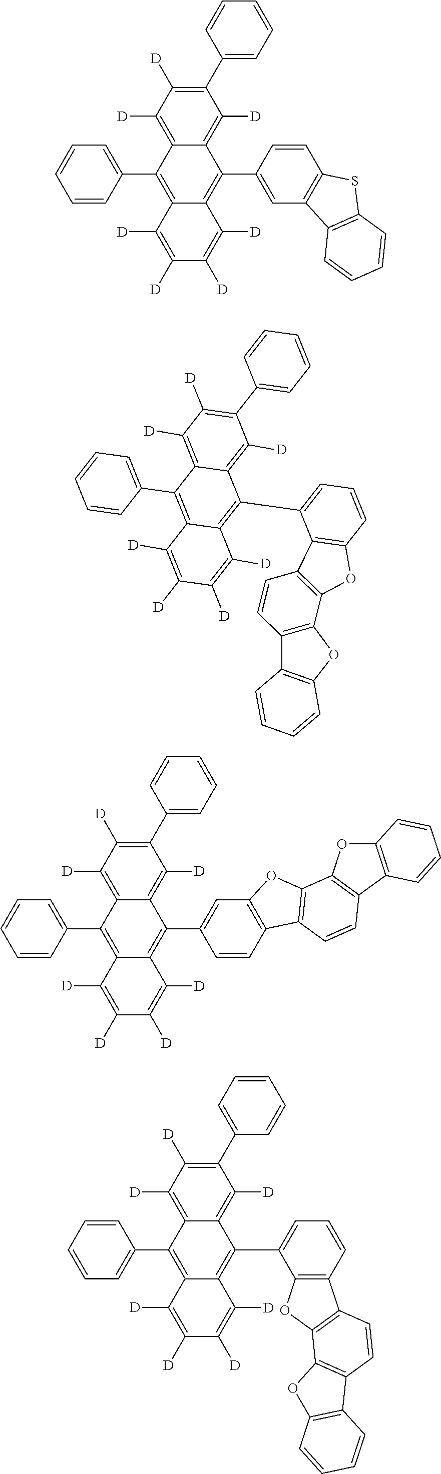 [SMILES: [2H]c1c([2H])c([2H])c2c(-c3ccc4sc5ccccc5c4c3)c3c([2H])c(-c4ccccc4)c([2H])c([2H])c3c(-c3ccccc3)c2c1[2H] ; [2H]c1c([2H])c([2H])c2c(-c3cccc4oc5c(ccc6c7ccccc7oc65)c34)c3c([2H])c(-c4ccccc4)c([2H])c([2H])c3c(-c3ccccc3)c2c1[2H] ; [2H]c1c([2H])c([2H])c2c(-c3ccc4c(c3)oc3c4ccc4c5ccccc5oc43)c3c([2H])c(-c4ccccc4)c([2H])c([2H])c3c(-c3ccccc3)c2c1[2H] ; [2H]c1c([2H])c([2H])c2c(-c3cccc4c3oc3c4ccc4c5ccccc5oc43)c3c([2H])c(-c4ccccc4)c([2H])c([2H])c3c(-c3ccccc3)c2c1[2H]]

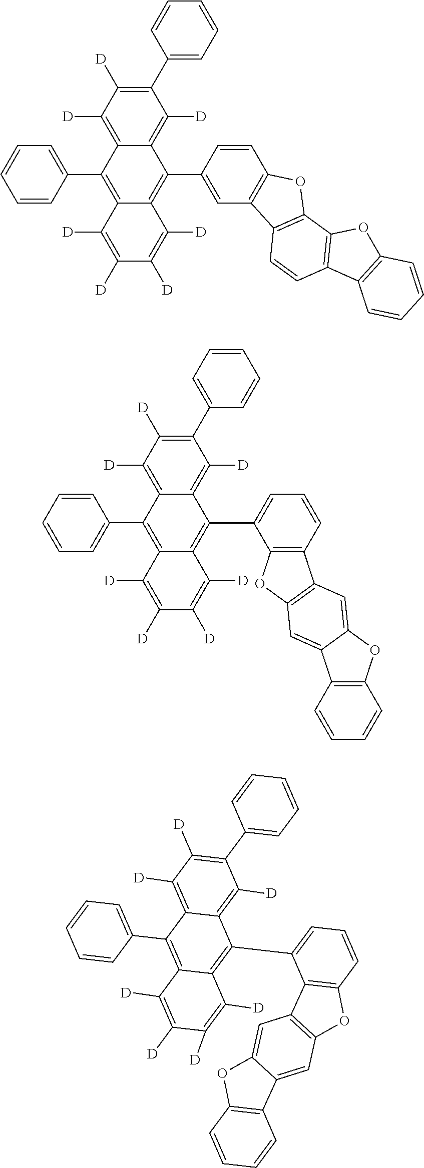 [SMILES: [2H]c1c([2H])c([2H])c2c(-c3ccc4oc5c(ccc6c7ccccc7oc65)c4c3)c3c([2H])c(-c4ccccc4)c([2H])c([2H])c3c(-c3ccccc3)c2c1[2H] ; [2H]c1c([2H])c([2H])c2c(-c3cccc4c3oc3cc5c(cc34)oc3ccccc35)c3c([2H])c(-c4ccccc4)c([2H])c([2H])c3c(-c3ccccc3)c2c1[2H] ; [2H]c1c([2H])c([2H])c2c(-c3cccc4oc5cc6c(cc5c34)oc3ccccc36)c3c([2H])c(-c4ccccc4)c([2H])c([2H])c3c(-c3ccccc3)c2c1[2H]]

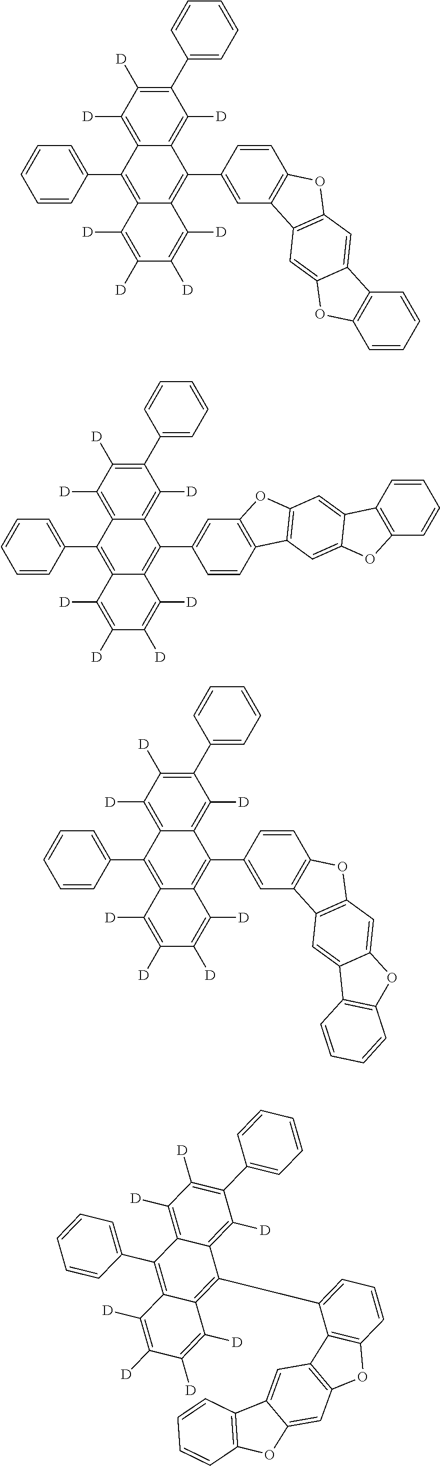 [SMILES: [2H]c1c([2H])c([2H])c2c(-c3ccc4oc5cc6c(cc5c4c3)oc3ccccc36)c3c([2H])c(-c4ccccc4)c([2H])c([2H])c3c(-c3ccccc3)c2c1[2H] ; [2H]c1c([2H])c([2H])c2c(-c3ccc4c(c3)oc3cc5c(cc34)oc3ccccc35)c3c([2H])c(-c4ccccc4)c([2H])c([2H])c3c(-c3ccccc3)c2c1[2H] ; [2H]c1c([2H])c([2H])c2c(-c3ccc4oc5cc6oc7ccccc7c6cc5c4c3)c3c([2H])c(-c4ccccc4)c([2H])c([2H])c3c(-c3ccccc3)c2c1[2H] ; [2H]c1c([2H])c([2H])c2c(-c3cccc4oc5cc6oc7ccccc7c6cc5c34)c3c([2H])c(-c4ccccc4)c([2H])c([2H])c3c(-c3ccccc3)c2c1[2H]]

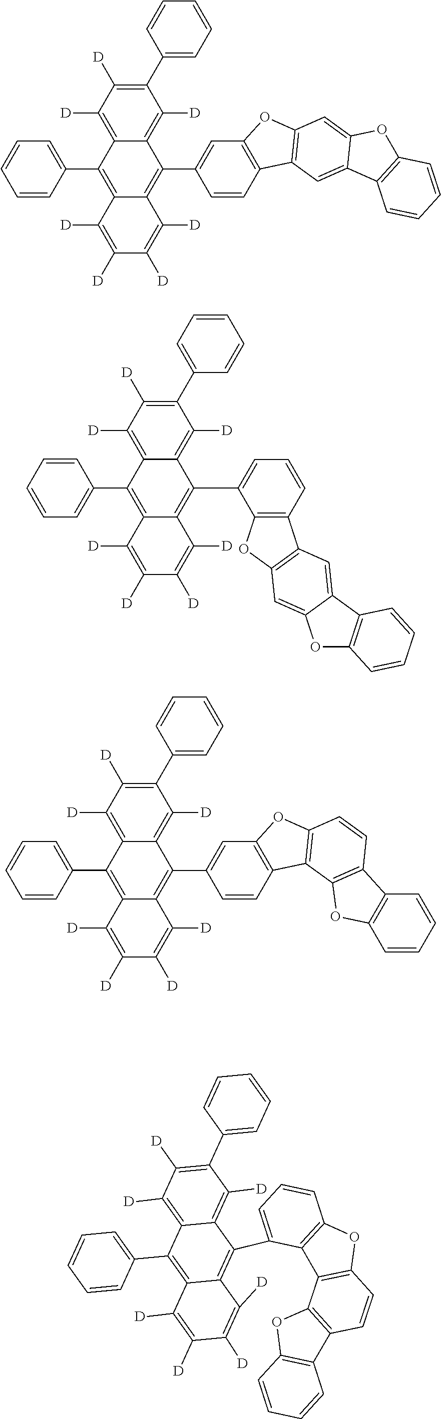

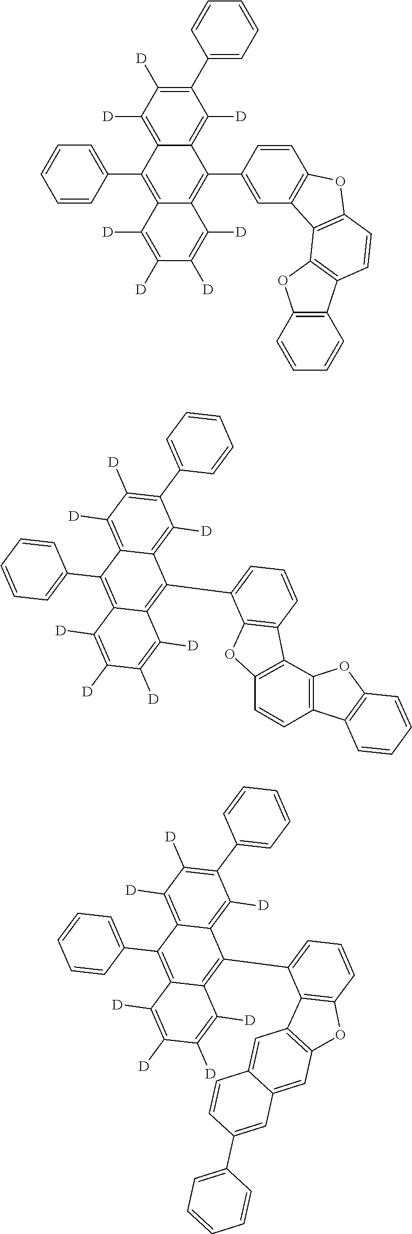 [SMILES: [2H]c1c([2H])c([2H])c2c(-c3ccc4oc5ccc6c7ccccc7oc6c5c4c3)c3c([2H])c(-c4ccccc4)c([2H])c([2H])c3c(-c3ccccc3)c2c1[2H] ; [2H]c1c([2H])c([2H])c2c(-c3cccc4c3oc3ccc5c6ccccc6oc5c34)c3c([2H])c(-c4ccccc4)c([2H])c([2H])c3c(-c3ccccc3)c2c1[2H] ; [2H]c1c([2H])c([2H])c2c(-c3cccc4oc5cc6cc(-c7ccccc7)ccc6cc5c34)c3c([2H])c(-c4ccccc4)c([2H])c([2H])c3c(-c3ccccc3)c2c1[2H]]

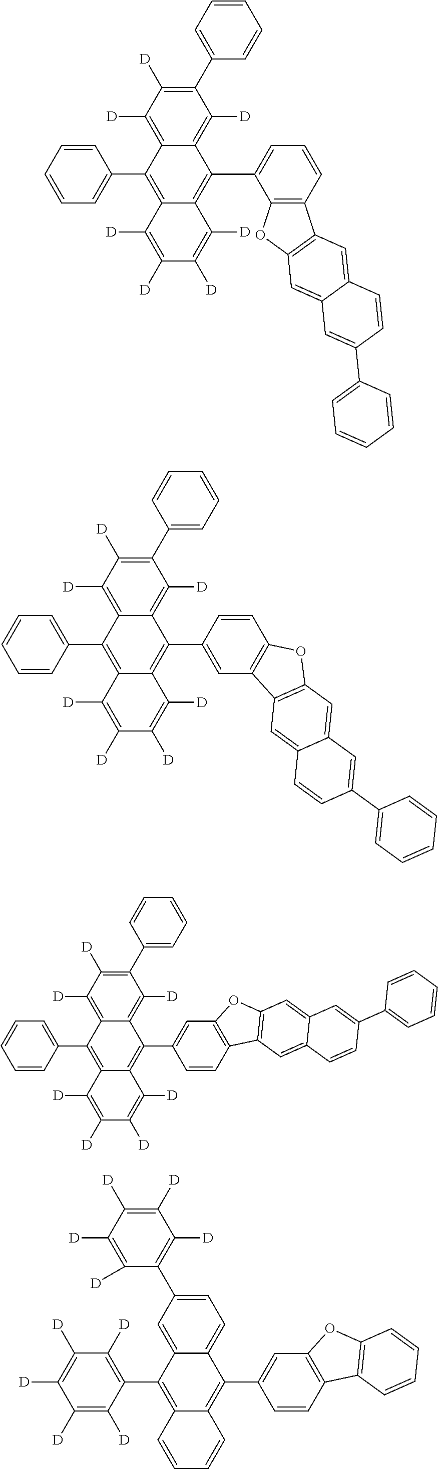

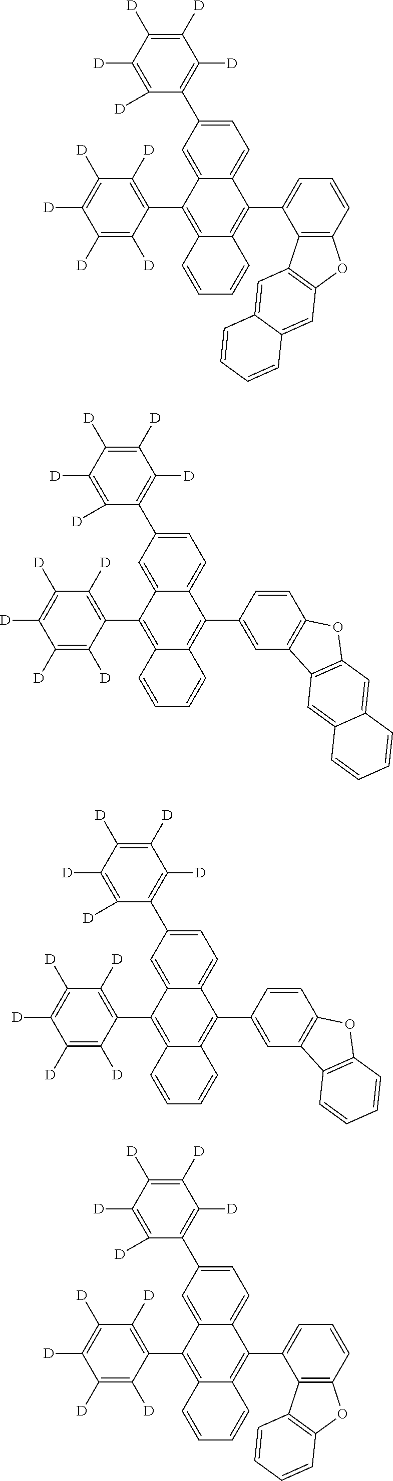 [SMILES: [2H]c1c([2H])c([2H])c(-c2ccc3c(-c4cccc5oc6cc7ccccc7cc6c45)c4ccccc4c(-c4c([2H])c([2H])c([2H])c([2H])c4[2H])c3c2)c([2H])c1[2H] ; [2H]c1c([2H])c([2H])c(-c2ccc3c(-c4ccc5oc6cc7ccccc7cc6c5c4)c4ccccc4c(-c4c([2H])c([2H])c([2H])c([2H])c4[2H])c3c2)c([2H])c1[2H] ; [2H]c1c([2H])c([2H])c(-c2ccc3c(-c4ccc5oc6ccccc6c5c4)c4ccccc4c(-c4c([2H])c([2H])c([2H])c([2H])c4[2H])c3c2)c([2H])c1[2H] ; [2H]c1c([2H])c([2H])c(-c2ccc3c(-c4cccc5oc6ccccc6c45)c4ccccc4c(-c4c([2H])c([2H])c([2H])c([2H])c4[2H])c3c2)c([2H])c1[2H]]

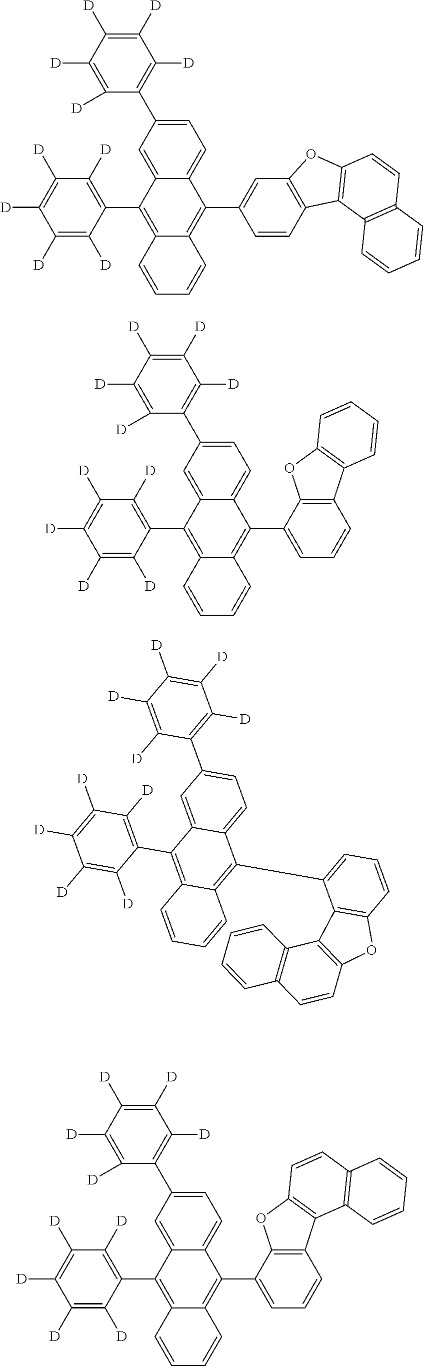

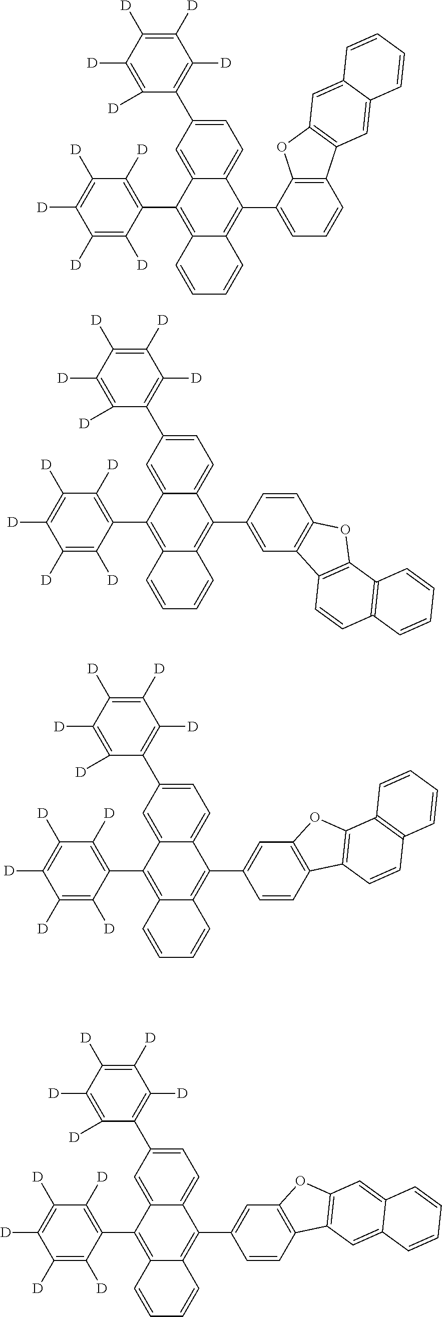

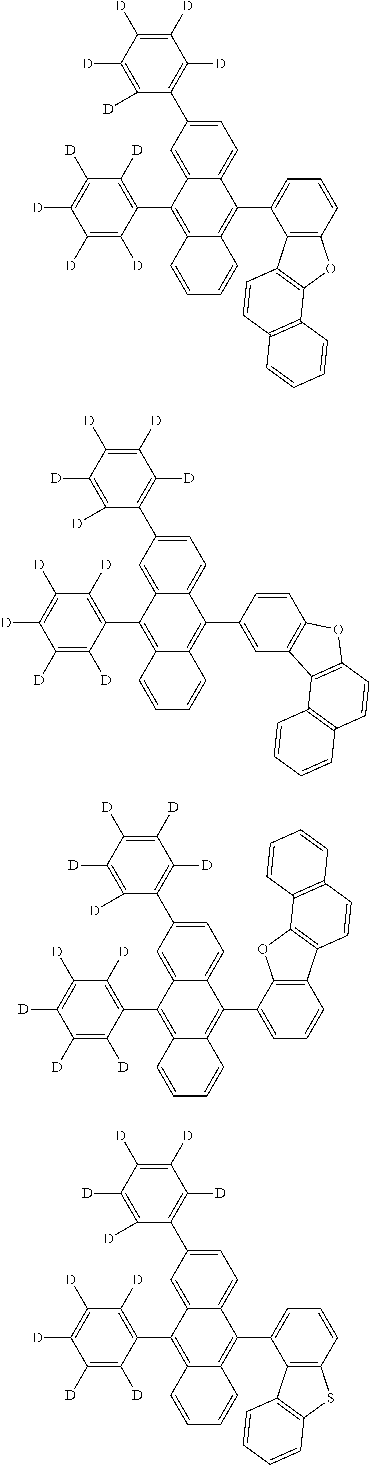

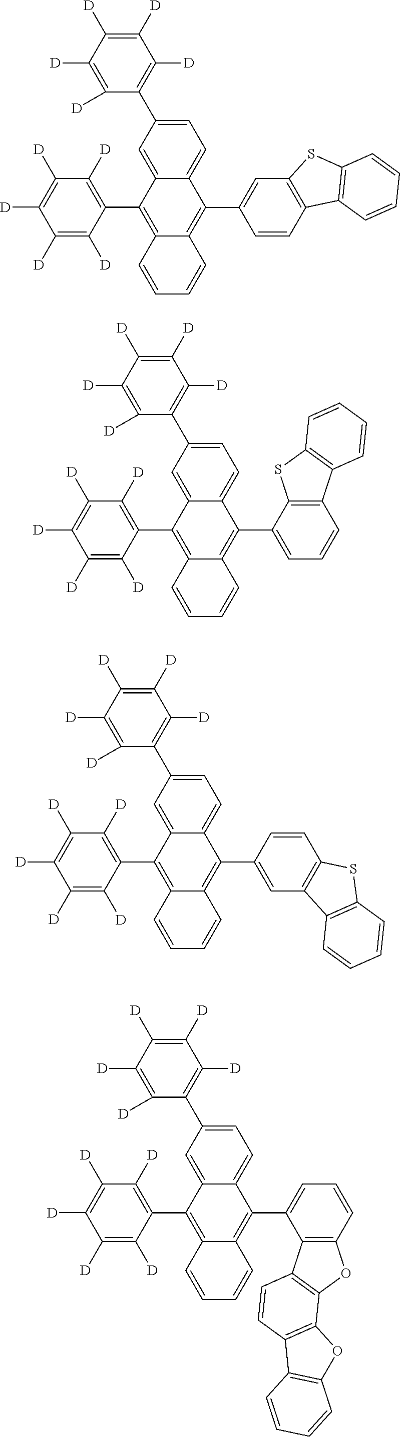

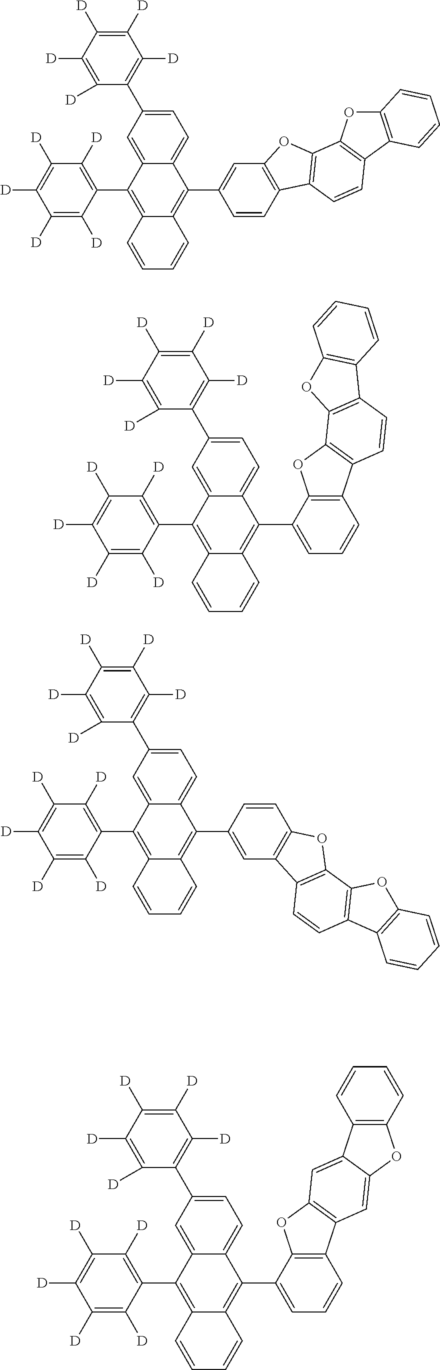

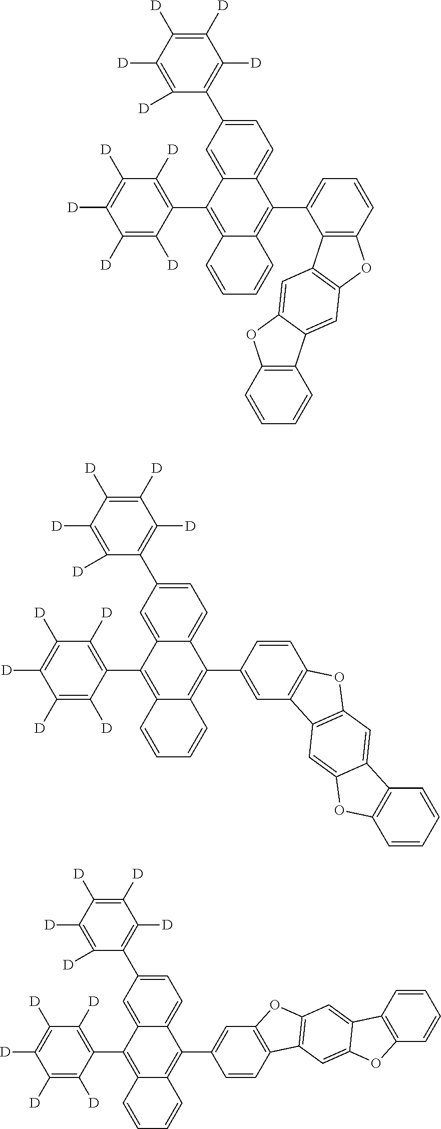 [SMILES: [2H]c1c([2H])c([2H])c(-c2ccc3c(-c4cccc5oc6cc7c(cc6c45)oc4ccccc47)c4ccccc4c(-c4c([2H])c([2H])c([2H])c([2H])c4[2H])c3c2)c([2H])c1[2H] ; [2H]c1c([2H])c([2H])c(-c2ccc3c(-c4ccc5oc6cc7c(cc6c5c4)oc4ccccc47)c4ccccc4c(-c4c([2H])c([2H])c([2H])c([2H])c4[2H])c3c2)c([2H])c1[2H] ; [2H]c1c([2H])c([2H])c(-c2ccc3c(-c4ccc5c(c4)oc4cc6c(cc45)oc4ccccc46)c4ccccc4c(-c4c([2H])c([2H])c([2H])c([2H])c4[2H])c3c2)c([2H])c1[2H]]

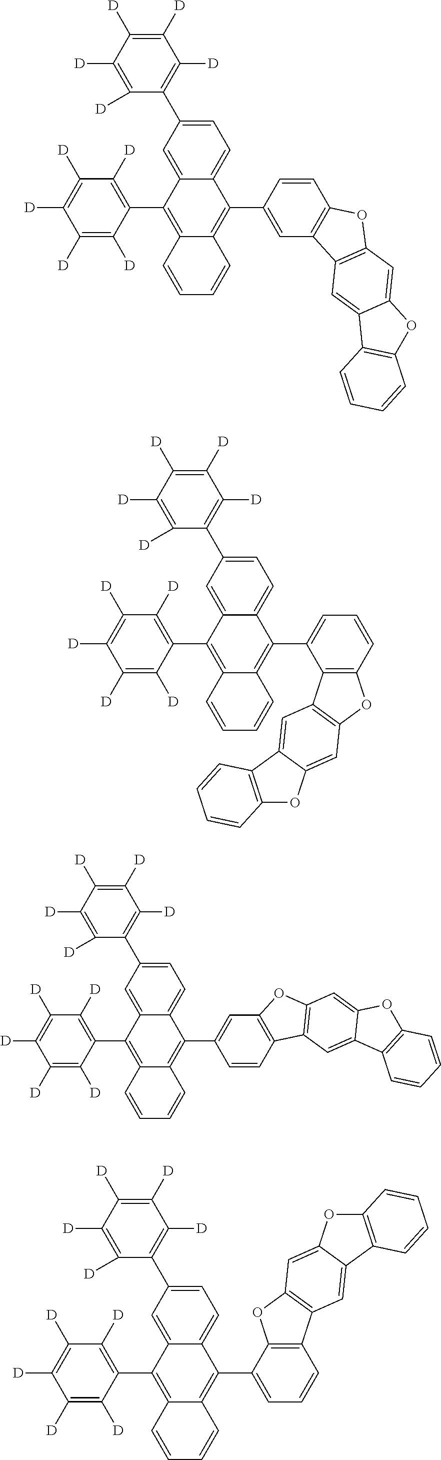

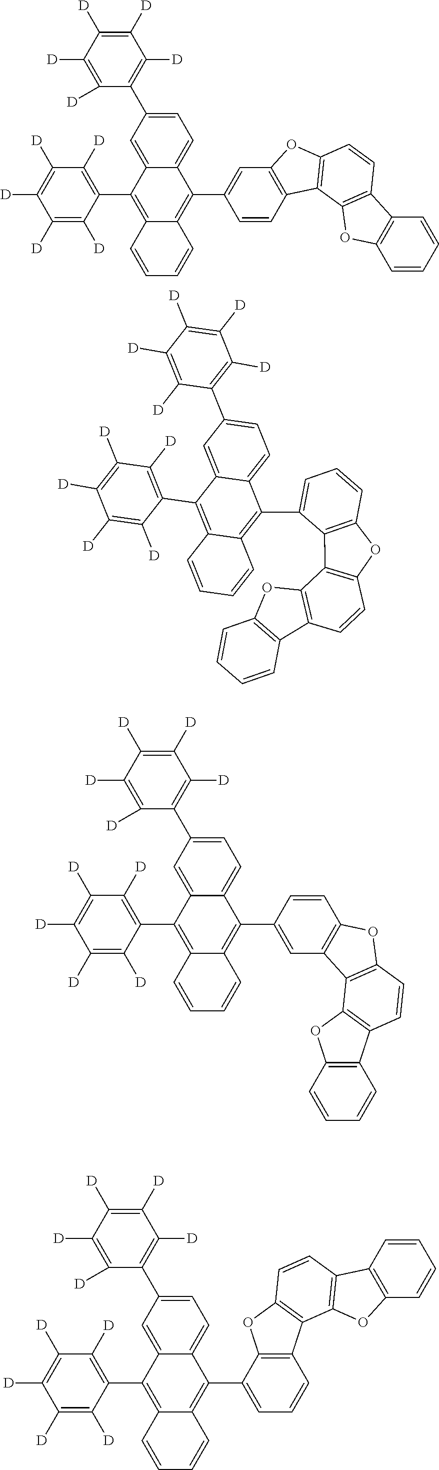

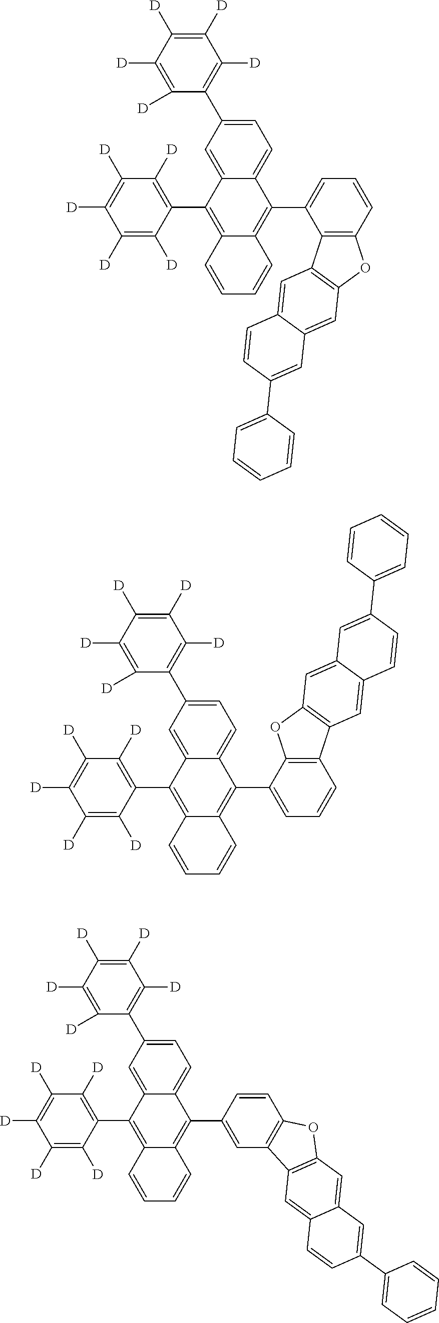 [SMILES: [2H]c1c([2H])c([2H])c(-c2ccc3c(-c4cccc5oc6cc7cc(-c8ccccc8)ccc7cc6c45)c4ccccc4c(-c4c([2H])c([2H])c([2H])c([2H])c4[2H])c3c2)c([2H])c1[2H] ; [2H]c1c([2H])c([2H])c(-c2ccc3c(-c4cccc5c4oc4cc6cc(-c7ccccc7)ccc6cc45)c4ccccc4c(-c4c([2H])c([2H])c([2H])c([2H])c4[2H])c3c2)c([2H])c1[2H] ; [2H]c1c([2H])c([2H])c(-c2ccc3c(-c4ccc5oc6cc7cc(-c8ccccc8)ccc7cc6c5c4)c4ccccc4c(-c4c([2H])c([2H])c([2H])c([2H])c4[2H])c3c2)c([2H])c1[2H]]

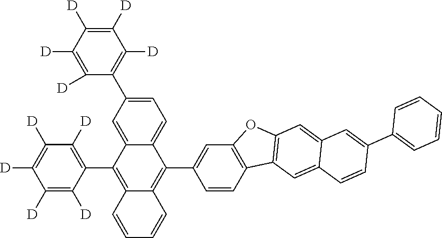 [SMILES: [2H]c1c([2H])c([2H])c(-c2ccc3c(-c4ccc5c(c4)oc4cc6cc(-c7ccccc7)ccc6cc45)c4ccccc4c(-c4c([2H])c([2H])c([2H])c([2H])c4[2H])c3c2)c([2H])c1[2H]]

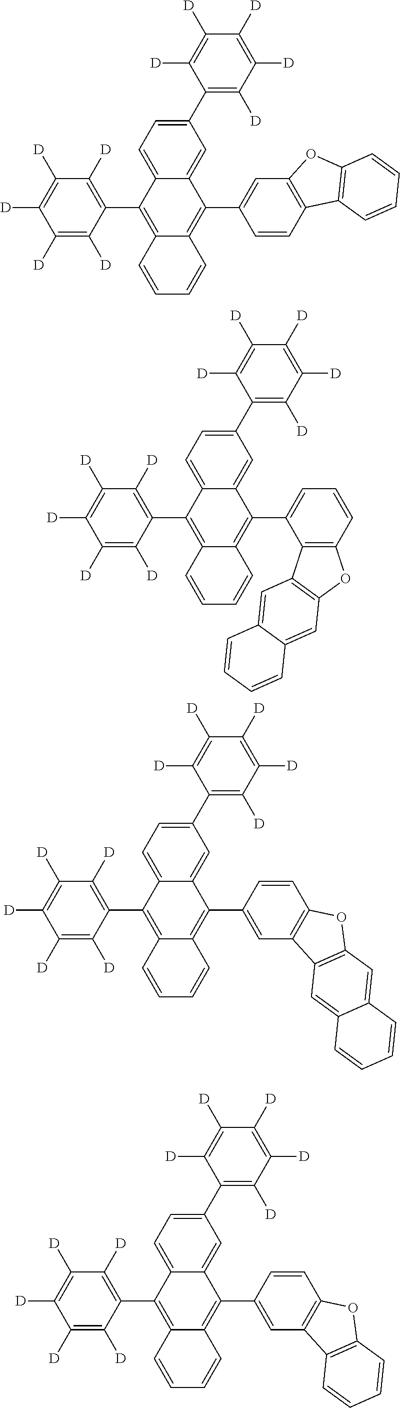

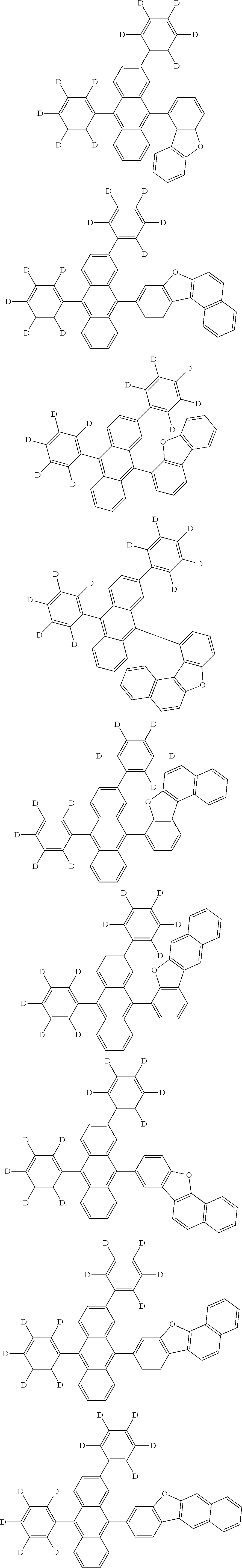

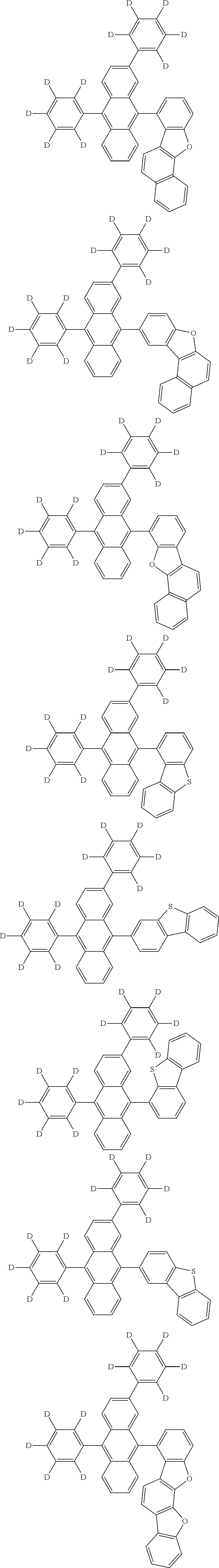

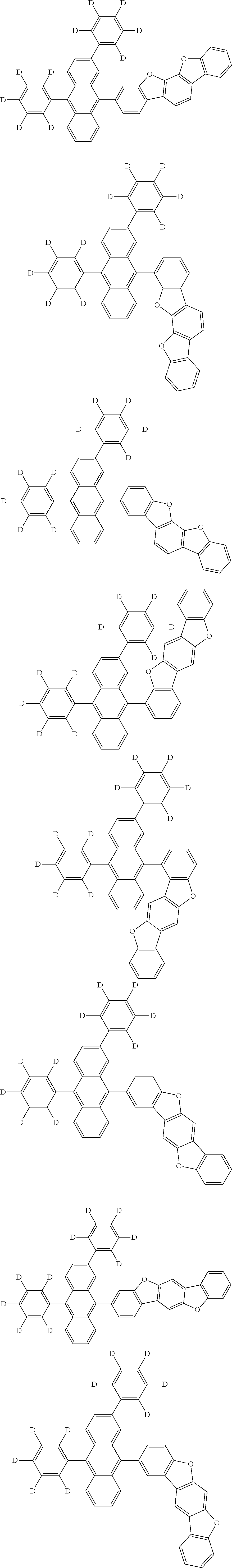

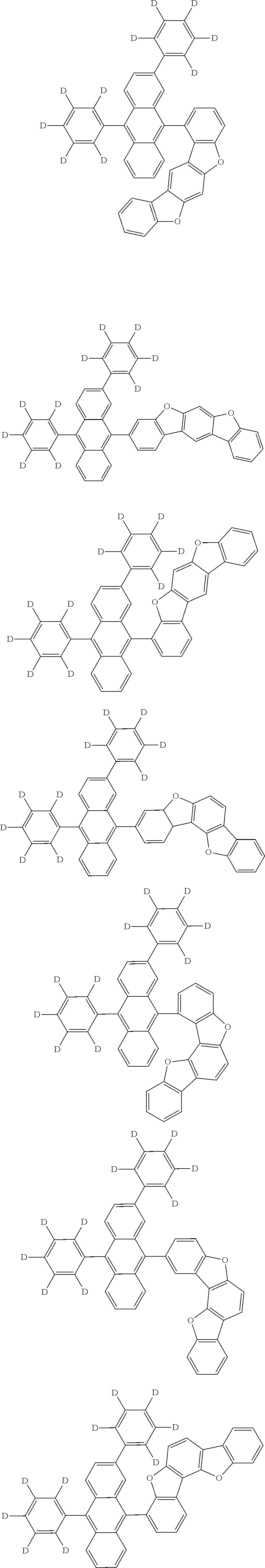

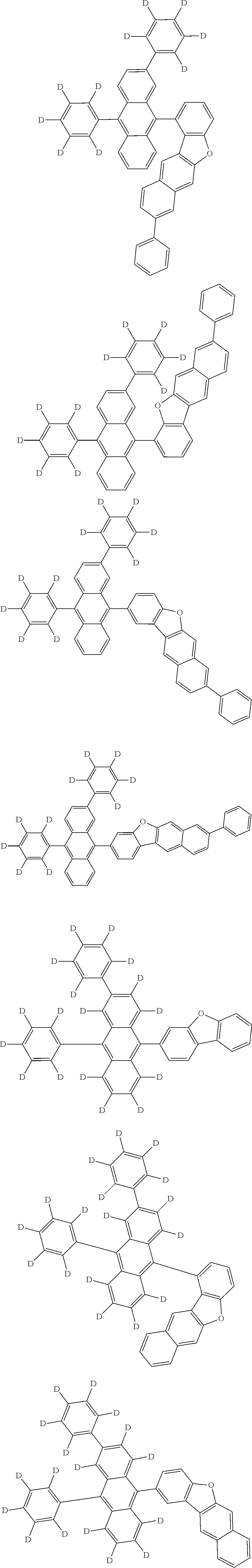

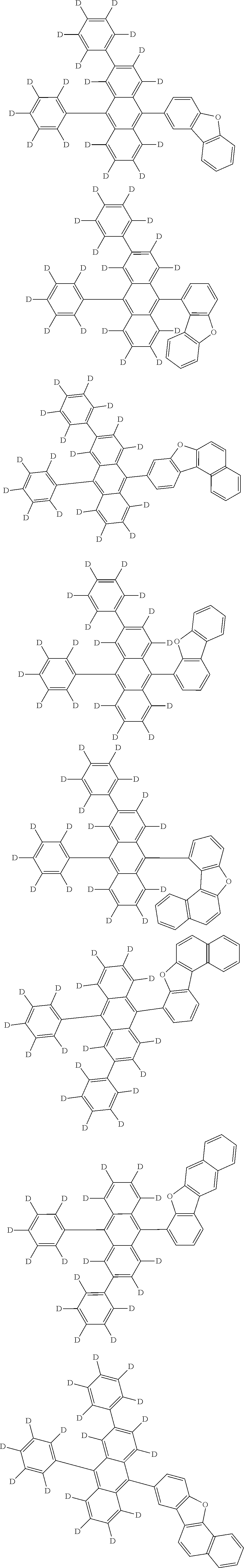

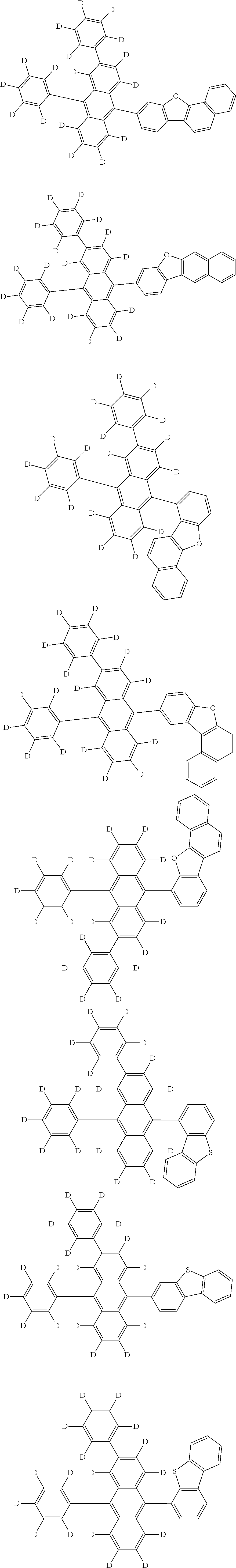

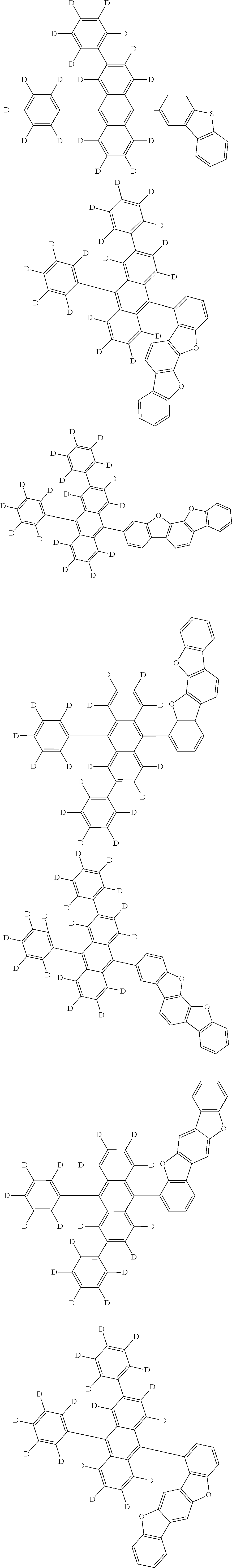

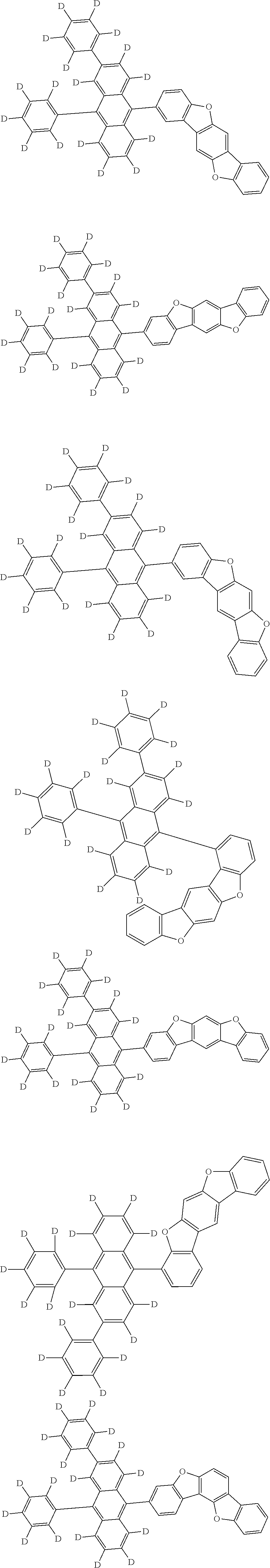

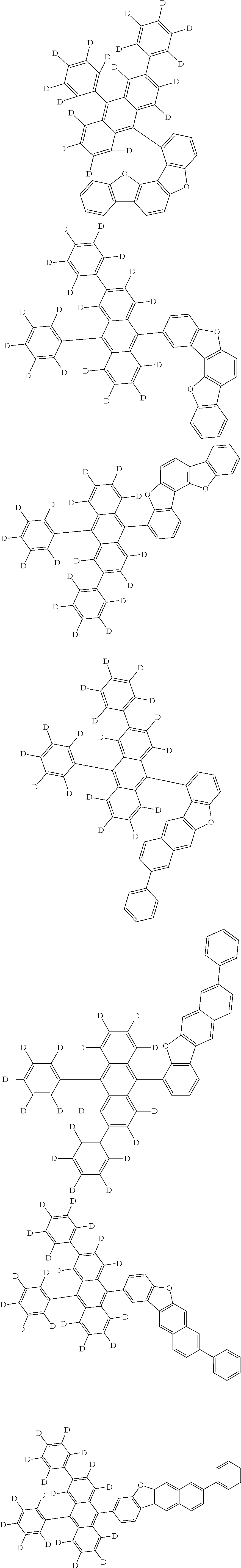

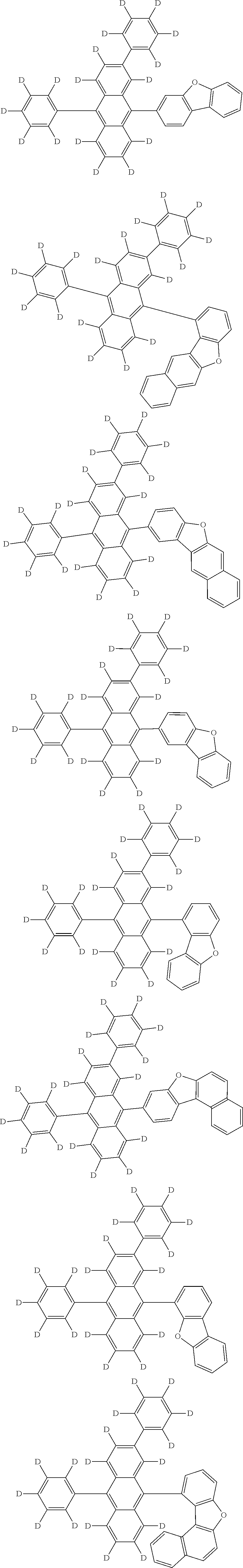

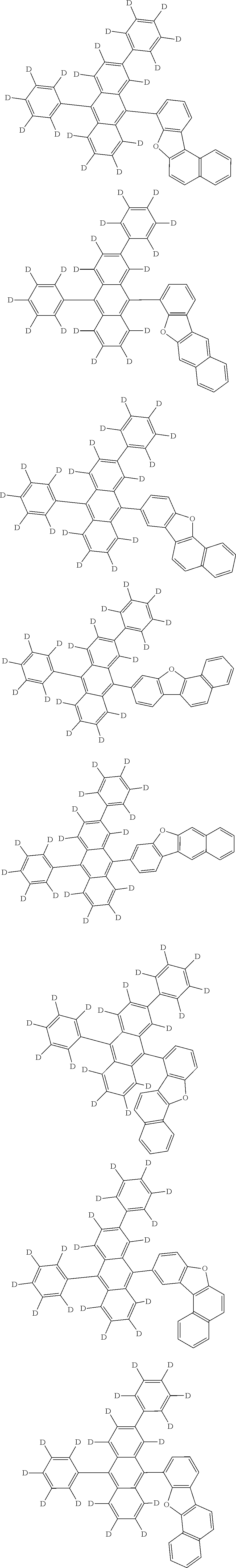

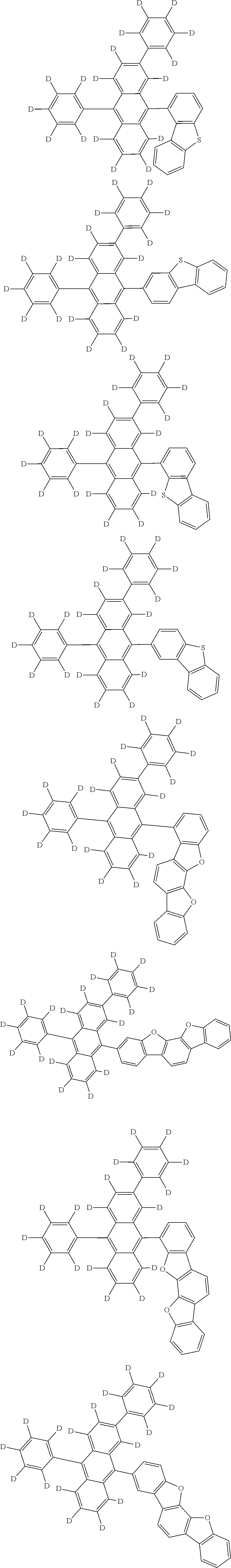

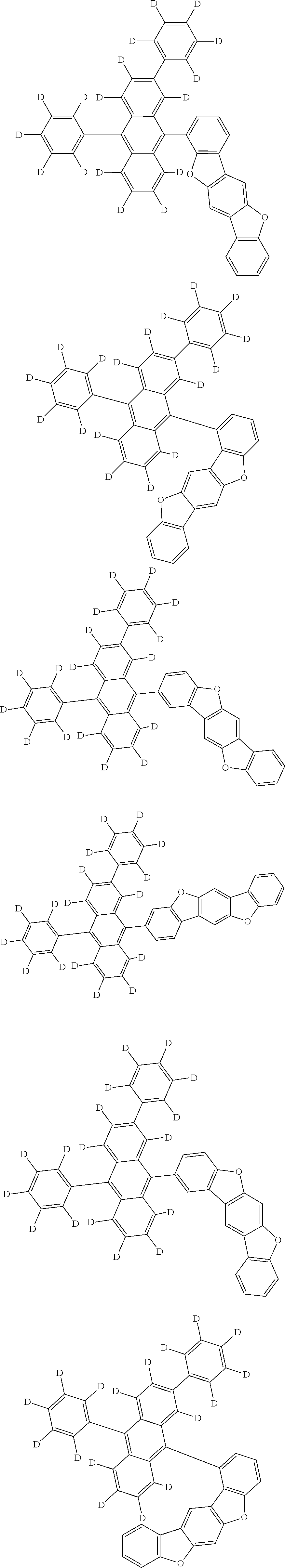

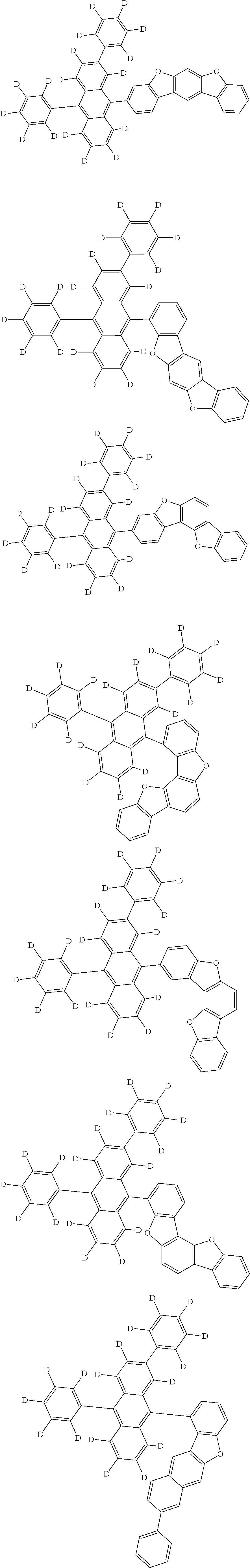

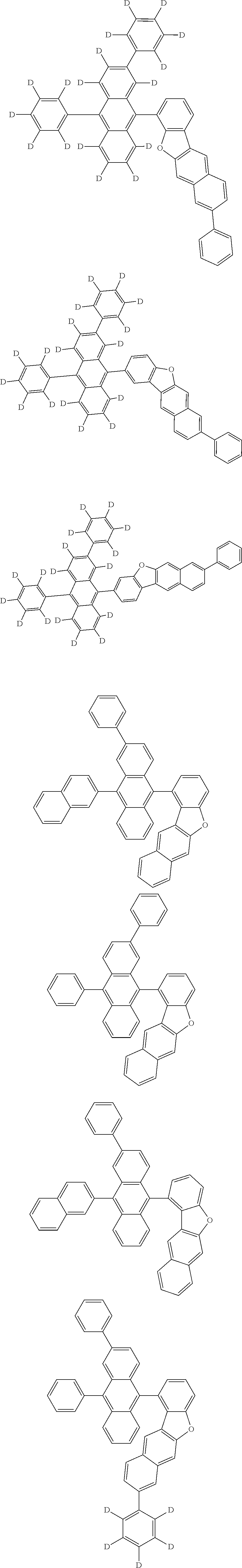

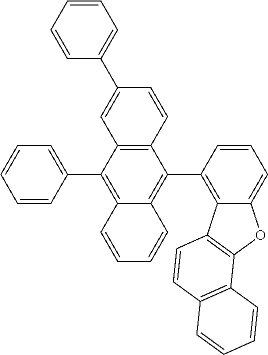 [SMILES: c1ccc(-c2ccc3c(-c4cccc5oc6c7ccccc7ccc6c45)c4ccccc4c(-c4ccccc4)c3c2)cc1]

### (Second Component)

The second component in the first organic EL device according to an aspect of the present invention is selected from the group consisting of an alkali metal, an alkali metal compound, an alkaline earth metal, an alkaline earth metal compound, a rare earth metal, a rare earth metal compound, an organic metal complex containing an alkali metal, an organic metal complex containing an alkaline earth metal, and an organic metal complex containing a rare earth metal.

Examples of the alkali metal include lithium, sodium, potassium, rubidium, cesium, and francium.

Examples of the alkaline earth metal include beryllium, magnesium, calcium, strontium, barium, and radium. In one embodiment, the alkaline earth metal is one or more metals selected from the group consisting of calcium, strontium, barium, and radium.

Examples of the rare earth metal include scandium, yttrium, lanthanum, cerium, praseodymium, neodymium, promethium, samarium, europium, gadolinium, terbium, dysprosium, holmium, erbium, thulium, ytterbium, and lutetium.

Examples of the alkali metal compound include alkali oxides such as Li₂O, Cs₂O and K₂O, and alkali halides such as LiF, NaF, CsF and KF, and the like.

Examples of the alkaline earth metal compound include BaO, SrO, CaO, and BaxSr₁₋ₓO (0<x<1), BaxCa₁₋ₓO (0<x<1) and the like as mixtures thereof.

Examples of the rare earth metal compound include YbF₃, ScF₃, ScO₃, Y₂O₃, Ce₂O₃, GdF₃, TbF₃, and the like.

The organic metal complex containing an alkali metal, the organic metal complex containing an alkaline earth metal, and the organic metal complex containing a rare earth metal are not particularly limited as long as at least one of an alkali metal ion, an alkaline earth metal ion, and a rare earth metal ion is contained as a metal ion. In addition, examples of a ligand include quinolinol, benzoquinolinol, acridinol, phenanthridinol, hydroxyphenyloxazole, hydroxyphenylthiazole, hydroxydiaryloxadiazole, hydroxydiarylthiadiazole, hydroxyphenylpyridine, hydroxyphenylbenzimidazole, hydroxybenzotriazole, hydroxyfluborane, bipyridyl, phenanthroline, phthalocyanine, porphyrin, cyclopentadiene, β-diketones, azomethines, derivatives thereof, and the like.

Examples of the organic metal complex containing an alkali metal include 8-hydroxyquinolinolato-lithium (Liq).

In one embodiment, the second component is selected from the group consisting of an alkali metal, an alkali metal compound, and an organic metal complex containing an alkali metal.

In one embodiment, the second component is lithium (Li), ytterbium (Yb), lithium fluoride (LiF), cesium fluoride (CsF), calcium fluoride (CaF₂), 8-hydroxyquinolinolato-lithium (Liq), or lithium oxide (LiOₓ).

The ratio of the first component and the second component is not particularly limited in a layer (hereinafter, also referred to as “layer A”) including the first component and the second component. In one embodiment, the amount of the first component is 30 to 70% by mass based on the total amount of the first component and the second component, and it may be 40 to 60% by mass.

The layer A may or may not include components other than the first component and the second component.

In one embodiment, the layer A substantially consists of the first component and the second component.

The phrase “substantially consists of the first component and the second component” means that the layer A does not include any other component, or it includes a trace amount of other components as long as the effects of the present invention are not impaired. For example, a case where other components are mixed as inevitable impurities corresponds to such a state.

In one embodiment, 80% by mass or more, 90% by mass or more, 95% by mass or more, 99% by mass or more, 99.5% by mass or more, 99.9% by mass or more, 99.99% by mass or more, or 100% by mass of the layer A is the first component and the second component.

In one embodiment, 80 mol % or more, 90 mol % or more, 95 mol % or more, 99 mol % or more, 99.5 mol % or more, 99.9 mol % or more, 99.99 mol % or more, or 100 mol % of the layer A is the first component and the second component.

In one embodiment, the layer A consists of the first component and the second component.

### [Second Organic EL Device]

The second organic EL device according to an aspect of the present invention includes a cathode, an anode, and one or two or more organic layers disposed between the cathode and the anode, wherein at least one layer of the one or two or more organic layers includes a first component and a second component.

The first component is a compound that satisfies formulas (R1) and (R2) to be described later and does not have structures represented by the formulas (M1) to (M4) in the molecule thereof.

The second component is selected from the group consisting of an alkali metal, an alkali metal compound, an alkaline earth metal, an alkaline earth metal compound, a rare earth metal, a rare earth metal compound, an organic metal complex containing an alkali metal, an organic metal complex containing an alkaline earth metal, and an organic metal complex containing a rare earth metal.

Since the second organic EL device according to an aspect of the present invention has the above-described configuration, higher device performance can be achieved. Specifically, it is possible to achieve an organic EL device having a low driving voltage and high efficiency even at a low current density.

As understood from the definition of each component, the first component and the second component are different from each other.

Hereinafter, each configuration of the second organic EL device according to an aspect of the present invention will be described.

### <First Component>

The first component in the second organic EL device according to an aspect of the present invention is a compound that satisfies the following formulas (R1) and (R2) and does not have the structures represented by the following formulas (M1) to (M4) in the molecule thereof:

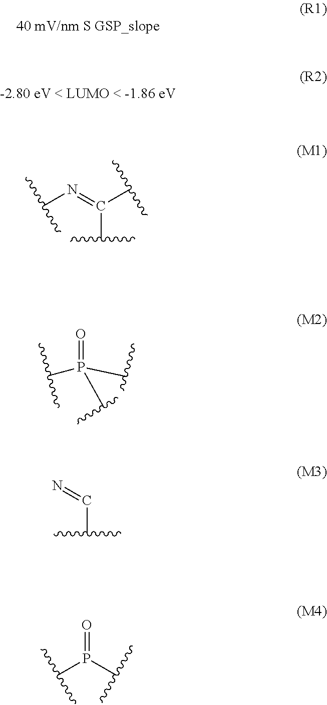

wherein in the formula (R1), GSP_slope represents a giant surface polarization slope; and in the formula (R2), LUMO represents a lowest unoccupied molecular orbital energy level.

### (Formula (R1): GSP_Slope)

GSP means giant surface polarization. As described later, GSP is proportional to the film thickness in principle. In order for handling as a numerical value independent of the film thickness, GSP_slope (giant surface polarization slope, GSP slope), which is a value obtained by dividing GSP by the film thickness, is used in the formula (R1). GSP_slope is measured by a method described in Examples.

GSP is a potential generated on a film surface by dipole moments of organic molecules slightly oriented in a film (layer) such as an organic layer forming an organic EL device, and is known to be proportional to the film thickness in principle. When the potential is generated on the film surface, an opposite charge is injected to cancel the potential, and thus it can be said that GSP is a physical property value that directly affects an amount of charge injection.

The amount of charge injection is one of important indices for optimizing the carrier balance between electrons and holes in each layer of the organic EL device and improving the performance of the device. Conventionally, ease of charge injection has been predicted for each layer of the organic EL device on the basis of an energy difference in lowest unoccupied molecular orbital energy level (LUMO) from an adjacent layer, but there are cases that are not explainable only by LUMO.

On the other hand, since GSP directly affects the amount of charge injection, it is considered that there is a correlation between GSP and the carrier balance. Therefore, the present inventors have conducted studies by introducing the above-described GSP_slope as a new physical property value related to the carrier balance, and as a result, have found that an amount of hole injection can be improved due to negative polarization occurring on the anode side when a compound exhibiting a certain level or more of GSP_slope is used for an organic EL device. In addition, it has also been found that the carrier balance in the low current density region is improved as a result, and the decrease in EQE is suppressed.

Many compounds that have the structures (electron-accepting structures) represented by the formulas (M1) to (M4) in molecules thereof and have been conventionally used as an electron-transporting material and the like of an organic EL device decrease EQE at a low current density. As the cause thereof, according to the studies of the present inventors, it has been considered that an amount of electron injection in the low current density region is excessive in these compounds, and there are insufficient holes to which the injected electrons are bonded, so that a carrier balance factor decreases, and EQE decreases. The relationship between the carrier balance and the external quantum efficiency (EQE) of the organic EL device is expressed by the following formula:

$$E Q E = γ \times X \times Φ \times η$$

wherein in the formula, γ represents the carrier balance factor, X represents the exciton generation probability (TTF efficiency), φ represents the luminescence quantum yield of a dopant material, and η represents the light extraction efficiency.

On the other hand, when the compound exhibiting a certain level or more of GSP_slope (compound satisfying the formula (R1)), which is different from the above-described conventional materials, is used, a sufficient amount of hole injection can be secured from the low current density due to its excellent hole injection capability, and the above-described problem of the decrease in EQE can be solved.

The upper limit of GSP_slope is not particularly limited, but, when GSP_slope is too large, there is a possibility that the amount of carrier injection is excessively increased, resulting in the decrease in EQE.

In one embodiment, the first component satisfies the following formula (R1-1):

$$GSP\_slope \leq 60 mV / nm \qquad ( R1 - 1 )$$

wherein in the formula (R1-1), GSP_slope is as defined in the formula (R1).

In one embodiment, the first component satisfies the following formula (R1-2):

$$41 mV / nm \leq GSP\_slope \qquad ( R1 - 2 )$$

wherein in the formula (R1-2), GSP_slope is as defined in the formula (R1).

In one embodiment, the first component satisfies the following formula (R1-3):

$$41 mV / nm \leq GSP\_slope \leq 60 mV / nm \qquad ( R1 - 3 )$$

wherein in the formula (R1-3), GSP_slope is as defined in the formula (R1).

In one embodiment, the first component satisfies the following formula (R1-4):

$$42 mV / nm \leq GSP\_slope \leq 51 mV / nm \qquad ( R1 - 4 )$$

wherein in the formula (R1-4), GSP_slope is as defined in the formula (R1).

In one embodiment, GSP_slope of the first component is 41 mV/nm or more, or 42 mV/nm or more.

In one embodiment, GSP_slope of the first component is 58 mV/nm or less, 56 mV/nm or less, 54 mV/nm or less, or 52 mV/nm or less.

### (Formula (R2): LUMO)

LUMO means the lowest unoccupied molecular orbital energy level. LUMO is measured by a method described in Examples.

When the compound satisfying the formula (R2) is used for at least one layer of organic layers of the organic EL device, an energy barrier at the time of injection of electrons into a layer adjacent to the layer can be reduced. As a result, the organic EL device having a low driving voltage can be achieved.

In one embodiment, the first component satisfies the following formula (R2-1):

$$- 2.6 eV \leq LUMO \leq - 2. eV \qquad ( R2 - 1 )$$

wherein in the formula (R2-1), LUMO is as defined in the formula (R2).

In one embodiment, the first component satisfies the following formula (R2-2):

$$- 2.26 eV \leq LUMO \leq - 2.09 eV \qquad ( R2 - 2 )$$

wherein in the formula (R2-2), LUMO is as defined in the formula (R2).

In one embodiment, LUMO of the first component is −1.90 eV or less, −1.95 eV or less, −2.00 eV or less, −2.05 eV or less, or −2.09 eV or less.

In one embodiment, LUMO of the first component is −2.80 eV or more, −2.70 eV or more, −2.60 eV or more, −2.50 eV or more, −2.40 eV or more, −2.30 eV or more, or −2.26 eV or more.

In one embodiment, the first component satisfies the formula (R1-3) and the formula (R2-2).

As for the phrase “does not have structures represented by the following formulas (M1) to (M4) in the molecule thereof”, the matters described in the first organic EL device according to an aspect of the present invention can be applied.

The first component in the second organic EL device according to an aspect of the present invention can be synthesized by using a known reaction or raw material suited for an intended product.

Hereinafter, specific examples of the first component in the second organic EL device according to an aspect of the present invention will be described, but these are merely examples, and the first component is not limited to the following specific examples.

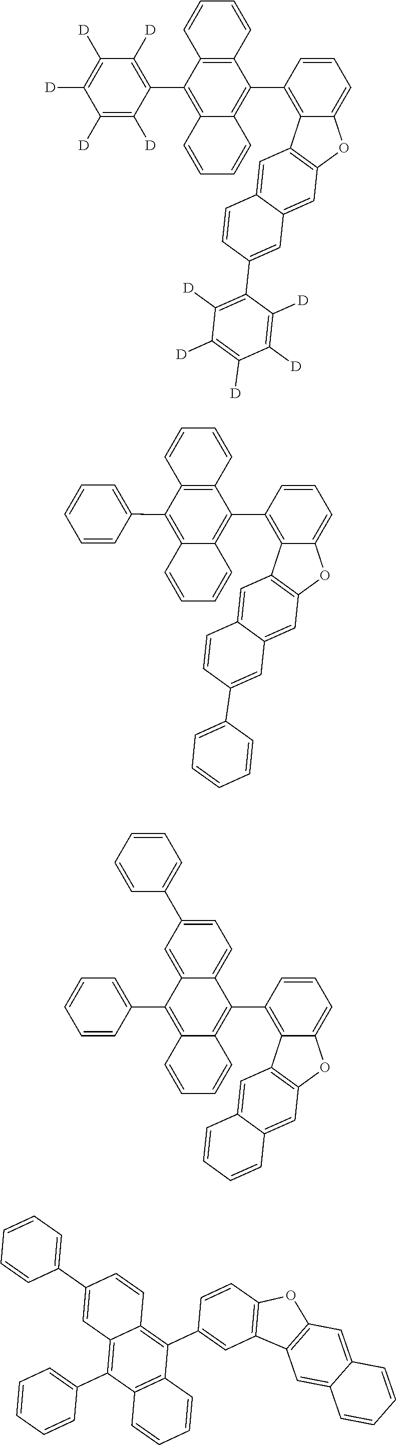

### <Second Component>

The second component in the second organic EL device according to an aspect of the present invention is selected from the group consisting of an alkali metal, an alkali metal compound, an alkaline earth metal, an alkaline earth metal compound, a rare earth metal, a rare earth metal compound, an organic metal complex containing an alkali metal, an organic metal complex containing an alkaline earth metal, and an organic metal complex containing a rare earth metal.

For the second component in the second organic EL device according to an aspect of the present invention, the matters described in the second component in the first organic EL device according to an aspect of the present invention can be applied.

The ratio between the first component and the second component in a layer (hereinafter, also referred to as “layer A”) including the first component and the second component is not particularly limited. For the layer A, the matters described in the first organic EL device according to an aspect of the present invention can be applied.

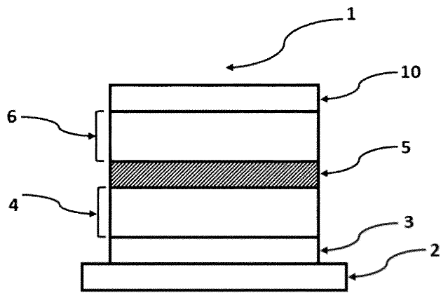

A schematic configuration of the organic EL device (the first organic EL device and the second organic EL device) according to an aspect of the present invention will be described with reference to FIG. 1.

An organic EL device 1 according to an aspect of the present invention includes a substrate 2, an anode 3, an emitting layer 5 which is an organic layer, a cathode 10, an organic layer 4 between the anode 3 and the emitting layer 5, and an organic layer 6 between the emitting layer 5 and the cathode 10.

Each of the organic layer 4 and the organic layer 6 may be a single layer, or it may include a plurality of layers.

In one embodiment, an organic EL device according to an aspect of the present invention includes an anode, an emitting layer, an electron-transporting zone, and a cathode in this order, wherein at least one layer in the electron-transporting zone includes the first component and the second component (includes a combination of the first component in the first organic EL device according to an aspect of the present invention and the second component in the first organic EL device according to an aspect of the present invention, or a combination of the first component in the second organic EL device according to an aspect of the present invention and the second component in the second organic EL device according to an aspect of the present invention).

### (Electron-Transporting Zone)

The electron-transporting zone is a generic term for one or two or more layers disposed between the emitting layer and the cathode. The electron-transporting zone includes, for example, layers referred to as a hole-blocking layer, an electron-transporting layer, and an electron-injecting layer, which will be described later, from the emitting layer side, and may have a stacked structure including all of these layers, or may have a layer configuration including only some of these layers. In addition, for each of the layers, two or more kinds of layers may be used, and for example, two kinds of electron-transporting layers having different compositions may be stacked.

Each layer may be formed using one kind of material alone, or may be formed using two or more kinds of materials in combination.

The stacked structure of the electron-transporting zone in the organic EL device according to an aspect of the present invention is exemplified below.
(a) (emitting layer/) first layer (electron-transporting layer)/second layer (electron-injecting layer) (/cathode)
(b) (emitting layer/) third layer (hole-blocking layer)/first layer (electron-transporting layer)/second layer (electron-injecting layer) (/cathode)
(c) (emitting layer/) third layer (hole-blocking layer)/fourth layer (first electron-transporting layer)/first layer (second electron-transporting layer)/second layer (electron-injecting layer) (/cathode)

In one embodiment, the electron-transporting zone includes at least a first layer and a second layer in this order from the emitting layer side, and the second layer includes the first component and the second component (includes a combination of the first component in the first organic EL device according to an aspect of the present invention and the second component in the first organic EL device according to an aspect of the present invention, or a combination of the first component in the second organic EL device according to an aspect of the present invention and the second component in the second organic EL device according to an aspect of the present invention).

In one embodiment, the second layer substantially does not include a compound having the structure represented by the formula (M1) in the molecule thereof, a compound having the structure represented by the formula (M2) in the molecule thereof, a compound having the structure represented by the formula (M3) in the molecule thereof, and a compound having the structure represented by the formula (M4) in the molecule thereof.

The phrase “substantially does not include” means that the second layer does not include any other component, or it includes a trace amount of other components as long as the effects of the present invention are not impaired. For example, the case of being mixed as inevitable impurities corresponds to such a state.

In one embodiment, the second layer substantially consists of the first component and the second component (substantially consists of the combination of the first component in the first organic EL device according to an aspect of the present invention and the second component in the first organic EL device according to an aspect of the present invention, or substantially consists of the combination of the first component in the second organic EL device according to an aspect of the present invention and the second component in the second organic EL device according to an aspect of the present invention).

The phrase “substantially consists of the first component and the second component” means that the second layer does not include any other component, or it includes a trace amount of other components as long as the effects of the present invention are not impaired. For example, a case where other components are mixed as inevitable impurities corresponds to such a state.

### (Other Configurations of Organic EL Device)

In the organic EL device according to an aspect of the present invention, as long as one or two or more organic layers disposed between the cathode and the anode satisfy the above-described conditions, conventionally known materials and device configurations can be applied as long as the effects of the present invention are not impaired.

Hereinafter, a device configuration, a material forming each layer, and the like in the organic EL device according to an aspect of the present invention will be described.

As the typical device configuration of the organic EL device, structures in which the following structures are stacked on a substrate are exemplified:
(1) Anode/emitting layer/electron-transporting zone/cathode,
(2) Anode/hole-transporting zone/emitting layer/electron-transporting zone/cathode,
wherein “/” indicates that layers are stacked adjacent to each other.

### (Hole-Transporting Zone)

The hole-transporting zone is a generic term for the one or two or more layers disposed between the anode and the emitting layer. The hole-transporting zone is configured, for example, from each layer which is referred to as an electron-blocking layer, a hole-transporting layer, and a hole-injecting layer described later from the emitting layer side, and it may have a stacked structure including all of them, or it may have a layer configuration merely including a part of them. In addition, each of the layers may be formed by using two or more kinds of layers, and for example, two kinds of hole-transporting layers having different compositions may be stacked.

Each layer may be formed using one kind of material alone, or it may be formed using two or more kinds of materials in combination.

Hereinafter, each layer of the organic EL device according to an aspect of the present invention will be described.

### (Substrate)

The substrate is used as a support of an emitting device. As the substrate, for example, glass, quartz, plastic, or the like can be used. In addition, a flexible substrate may be used. The flexible substrate is a bendable (flexible) substrate, and examples thereof include plastic substrates made of polycarbonate and polyvinyl chloride, or the like.

### (Anode)

For the anode formed on the substrate, a metal, an alloy, an electrically conductive compound, a mixture thereof, or the like having a large work function (specifically, 4.0 eV or more) is preferably used. Specific examples thereof include indium oxide-tin oxide (ITO: Indium Tin Oxide), indium oxide-tin oxide containing silicon or silicon oxide, indium oxide-zinc oxide, tungsten oxide, indium oxide containing zinc oxide, graphene, and the like. Gold (Au), platinum (Pt), a nitride of a metal material (for example, titanium nitride), and the like are exemplified.

### (Hole-Injecting Layer)

The hole-injecting layer is a layer containing a substance having a high hole injectability. As the substance having a high hole injectability, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, a polymer compound (such as an oligomer, a dendrimer, or a polymer), or the like can also be used.

### (Hole-Transporting Layer)

The hole-transporting layer is a layer containing a substance having a high hole transportability. For the hole-transporting layer, an aromatic amine compound, a carbazole derivative, an anthracene derivative, or the like can be used. A polymer compound such as poly(N-vinylcarbazole) (abbreviation: PVK) or poly(4-vinyltriphenylamine) (abbreviation: PVTPA) can also be used. However, a substance other than these substances may be used as long as the substance has a higher hole transportability than an electron transportability. The layer containing a substance having a high hole transportability may be not only a single layer but also layers in which two or more layers formed of the above substances are stacked.

### (Guest (Dopant) Material of Emitting Layer)

The emitting layer is a layer containing a substance having high emitting property, and various materials can be used. For example, a fluorescent compound that emits fluorescence or a phosphorescent compound that emits phosphorescence can be used as the substance having high emitting property. The fluorescent compound is a compound capable of emitting light from a singlet excited state, and the phosphorescent compound is a compound capable of emitting light from a triplet excited state.

As a blue fluorescent emitting material that can be used for the emitting layer, a pyrene derivative, a styrylamine derivative, a chrysene derivative, a fluoranthene derivative, a fluorene derivative, a diamine derivative, a triarylamine derivative, or the like can be used. As a green fluorescent emitting material that can be used for the emitting layer, an aromatic amine derivative or the like can be used. As a red fluorescent emitting material that can be used for the emitting layer, a tetracene derivative, a diamine derivative, or the like can be used.

As a blue phosphorescent emitting material that can be used for the emitting layer, a metal complex such as an iridium complex, an osmium complex, or a platinum complex is used. As a green phosphorescent emitting material that can be used for the emitting layer, an iridium complex or the like is used. As a red phosphorescent emitting material that can be used for the emitting layer, a metal complex such as an iridium complex, a platinum complex, a terbium complex, or a europium complex is used.

### (Host Material of Emitting Layer)

The emitting layer may have a configuration in which the above-described substance having high emitting property (guest material) is dispersed in another substance (host material). As a substance for dispersing the substance having high emitting property, various substances can be used, and it is preferable to use a substance having a higher lowest unoccupied molecular orbital level (LUMO level) and a lower highest occupied molecular orbital level (HOMO level) than the substance having high emitting property.

As the substance (host material) for dispersing the substance having high emitting property, 1) a metal complex such as an aluminum complex, a beryllium complex, or a zinc complex, 2) a heterocyclic compound such as an oxadiazole derivative, a benzimidazole derivative, or a phenanthroline derivative, 3) a fused aromatic compound such as a carbazole derivative, an anthracene derivative, a phenanthrene derivative, a pyrene derivative, or a chrysene derivative, and 4) an aromatic amine compound such as a triarylamine derivative or a fused polycyclic aromatic amine derivative are used.

In addition, a compound having delayed fluorescence (thermally activated delayed fluorescence) can also be used as the host material. It is also preferable that the emitting layer contains the materials used in the present invention described above and a host compound having delayed fluorescence.

### (Electron-Blocking Layer, Hole-Blocking Layer, and Exciton-Blocking Layer)

An electron-blocking layer, a hole-blocking layer, an exciton (triplet)-blocking layer, or the like may be provided adjacent to the emitting layer.

The electron-blocking layer is a layer having a function of preventing leakage of electrons from the emitting layer to the hole-transporting layer. The hole-blocking layer is a layer having a function of preventing leakage of holes from the emitting layer to the electron-transporting layer. The exciton-blocking layer is a layer having a function of preventing diffusion of excitons generated in the emitting layer to the adjacent layers and confining the excitons within the emitting layer.

### (Electron-Transporting Layer)

The electron-transporting layer is a layer containing a substance having a high electron transportability. For the electron-transporting layer, 1) a metal complex such as an aluminum complex, a beryllium complex, or a zinc complex, 2) a heteroaromatic compound such as an imidazole derivative, a benzimidazole derivative, an azine derivative, a carbazole derivative, or a phenanthroline derivative, and 3) a polymer compound can be used.

### (Electron-Injecting Layer)

The electron-injecting layer is a layer containing a substance having a high electron injectability. For the electron-injecting layer, a metal complex compound such as lithium (Li), ytterbium (Yb), lithium fluoride (LiF), cesium fluoride (CsF), calcium fluoride (CaF₂), or 8-hydroxyquinolinolato-lithium (Liq), an alkali metal such as lithium oxide (LiOₓ), an alkaline earth metal, or a compound thereof can be used.

### (Cathode)

For the cathode, a metal, an alloy, an electrically conductive compound, a mixture thereof, or the like having a small work function (specifically, 3.8 eV or less) is preferably used. Specific examples of such a cathode material include elements belonging to Group 1 or Group 2 of the Periodic Table of the Elements, that is, alkali metals such as lithium (Li) and cesium (Cs), alkaline earth metals such as magnesium (Mg), calcium (Ca), and strontium (Sr), alloys containing these metals (for example, MgAg and AlLi), rare earth metals such as europium (Eu) and ytterbium (Yb), alloys containing these metals, and the like.

In the organic EL device according to an aspect of the present invention, the film thickness of each layer is not particularly limited, but is generally preferably in a range of several nm to 1 μm in order to suppress defects such as pinholes, suppress the applied voltage to be low, and improve light emission efficiency.

### [Method for Fabricating Organic EL Device]

In the organic EL device according to an aspect of the present invention, a method for forming each layer is not particularly limited. A conventionally known method for forming each layer by a vacuum deposition method, a spin coating method, or the like can be used. Each layer such as the emitting layer can be formed by a known method such as a vacuum deposition method, a molecular beam deposition method (MBE method) or a coating method, such as a dipping method, a spin coating method, a casting method, a bar coating method, or a roll coating method using a solution prepared by dissolving the material in a solvent.

### [First Composition]

A first composition according to an aspect of the present invention includes a first component and a second component.

The first component is a compound represented by the above formula (1).

The second component is selected from the group consisting of an alkali metal, an alkali metal compound, an alkaline earth metal, an alkaline earth metal compound, a rare earth metal, a rare earth metal compound, an organic metal complex containing an alkali metal, an organic metal complex containing an alkaline earth metal, and an organic metal complex containing a rare earth metal.

The first component and the second component in the first composition according to an aspect of the present invention are as described in the first organic EL device according to an aspect of the present invention described above.

A form of the composition is not particularly limited, and examples thereof include a solid, a solution, a film (layer), and the like. Examples of the film (layer) include an organic layer (for example, a hole-blocking layer, an electron-transporting layer, and an electron-injecting layer) forming an organic EL device.

### [Second Composition]

A second composition according to an aspect of the present invention includes a first component and a second component.

The first component is a compound that satisfies the above formulas (R1) and (R2) and does not have the structures represented by the above formulas (M1) to (M4) in the molecule thereof.

The second component is selected from the group consisting of an alkali metal, an alkali metal compound, an alkaline earth metal, an alkaline earth metal compound, a rare earth metal, a rare earth metal compound, an organic metal complex containing an alkali metal, an organic metal complex containing an alkaline earth metal, and an organic metal complex containing a rare earth metal.

The first component and the second component in the second composition according to an aspect of the present invention are as described in the second organic EL device according to an aspect of the present invention described above.

A form of the composition is not particularly limited, and examples thereof include a solid, a solution, a film (layer), and the like. Examples of the film (layer) include an organic layer (for example, a hole-blocking layer, an electron-transporting layer, and an electron-injecting layer) forming an organic EL device.

### [Electronic Apparatus]

An electronic apparatus according to an aspect of the present invention includes the organic EL device according to an aspect of the present invention or a third organic EL device to be described later.

Specific examples of the electronic apparatus include display components such as an organic EL panel module, display devices for a television, a mobile phone, a personal computer and the like, emitting devices such as a light and a vehicular lamp, and the like.

### [Novel Compound]

A compound according to an aspect of the present invention is a compound represented by the following formula (2):

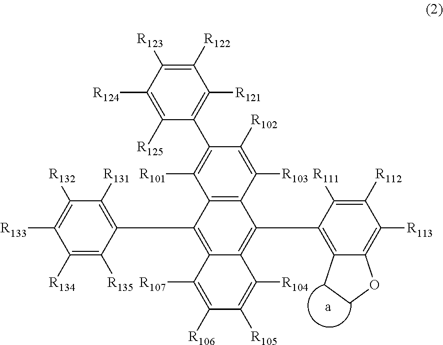

wherein in the formula (2),
a ring a is
a substituted or unsubstituted aromatic hydrocarbon ring having 10 to 50 ring carbon atoms, or
a substituted or unsubstituted heterocyclic ring having 6 to 50 ring atoms;
R₁₀₁ to R₁₀₇, R₁₁₁ to R₁₁₃, R₁₂₁ to R₁₂₅, and R₁₃₁ to R₁₃₅ are independently a hydrogen atom, or a substituent R,
provided that at least one of R₁₁₁ to R₁₁₃ is the substituent R, or the ring a has at least one substituent;
the substituent R is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,

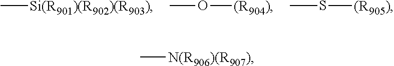

a halogen atom, a cyano group, a nitro group,
a substituted or unsubstituted aryl group having 6 to 50 ring carbon atoms, or
a substituted or unsubstituted monovalent heterocyclic group having 5 to 50 ring atoms;
R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more of each of R₉₀₁ to R₉₀₇ are present, the two or more of each of R₉₀₁ to R₉₀₇ may be the same as or different from each other; and
when two or more substituents R are present, the two or more substituents R may be the same as or different from each other.

In the formula (2), the ring a forms a fused ring with a benzofuran skeleton. That is, the ring a has at least one carbon-carbon bond, and shares the carbon-carbon bond to the benzofuran skeleton.

In one embodiment, the ring a includes a six-membered ring structure having at least one carbon-carbon bond, the carbon-carbon bond being shared with the benzofuran skeleton.

In one embodiment, the compound represented by the formula (2) is a compound represented by the following formula (2-1):

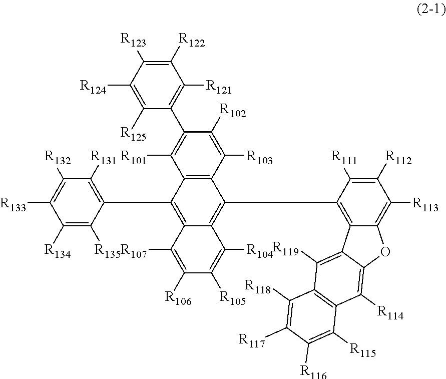

wherein in the formula (2-1),
R₁₀₁ to R₁₀₇, R₁₁₁ to R₁₁₉, R₁₂₁ to R₁₂₅, and R₁₃₁ to R₁₃₅ are independently a hydrogen atom, or a substituent R; provided that at least one of R₁₁₁ to R₁₁₉ is the substituent R; and
the substituent R is as defined in the formula (2).

In one embodiment, the compound represented by the formula (2) is a compound represented by the following formula (2-11):

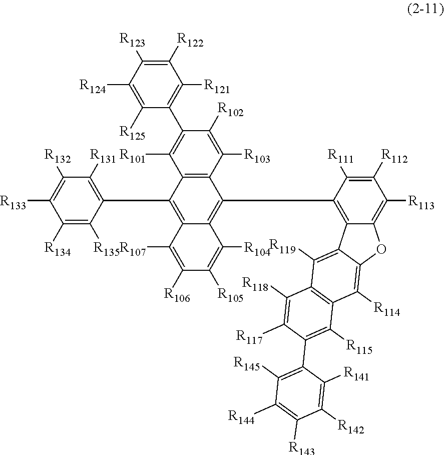

wherein in the formula (2-11),
R₁₀₁ to R₁₀₇, R₁₁₁ to R₁₁₅, R₁₁₇ to R₁₁₉, R₁₂₁ to R₁₂₅, R₁₃₁ to R₁₃₅, and R₁₄₁ to R₁₄₅ are independently a hydrogen atom, or a substituent R; and
the substituent R is as defined in the formula (2).

In one embodiment, the substituent R is a substituted or unsubstituted aryl group having 6 to 50 ring carbon atoms.

In one embodiment, the substituent R is an unsubstituted aryl group having 6 to 50 ring carbon atoms.

In one embodiment, R₁₀₁ to R₁₀₇, R₁₁₁ to R₁₁₅, R₁₁₇ to R₁₁₉, R₁₂₁ to R₁₂₅, R₁₃₁ to R₁₃₅, and R₁₄₁ to R₁₄₅ are hydrogen atoms.

In one embodiment, the compound represented by the formula (2) contains at least one deuterium atom.

In one embodiment, one or more hydrogen atoms selected from the group consisting of
R₁₀₁ to R₁₀₇ which are hydrogen atoms,
a hydrogen atom of R₁₀₁ to R₁₀₇ which are the substituents R,
R₁₁₁ to R₁₁₃ which are hydrogen atoms,
a hydrogen atom of R₁₁₁ to R₁₁₃ which are the substituents R,
R₁₂₁ to R₁₂₅ which are hydrogen atoms,
a hydrogen atom of R₁₂₅ to R₁₂₅ which are the substituents R,
R₁₃₁ to R₁₃₅ which are hydrogen atoms,
a hydrogen atom of R₁₃₁ to R₁₃₅ which are the substituents R, and
a hydrogen atom of the ring a (including a hydrogen atom of the substituent of the ring a) are deuterium atoms.

In one embodiment, a substituent in the case of “substituted or unsubstituted” in the formula (2) is selected from the group consisting of
an alkyl group having 1 to 50 carbon atoms,
a haloalkyl group having 1 to 50 carbon atoms,
an alkenyl group having 2 to 50 carbon atoms,
an alkynyl group having 2 to 50 carbon atoms,
a cycloalkyl group having 3 to 50 ring carbon atoms,
an alkoxy group having 1 to 50 carbon atoms,
an alkylthio group having 1 to 50 carbon atoms,
an aryloxy group having 6 to 50 ring carbon atoms,
an arylthio group having 6 to 50 ring carbon atoms,
an aralkyl group having 7 to 50 carbon atoms,

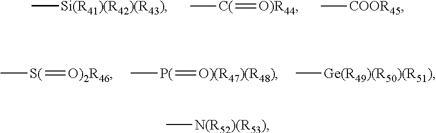

a hydroxy group,
a halogen atom,
a cyano group,
a nitro group,
an aryl group having 6 to 50 ring carbon atoms, and
a monovalent heterocyclic group having 5 to 50 ring atoms.

R₄₁ to R₄₃, R₄₆, and R₄₉ to R₅₃ are independently a hydrogen atom, an alkyl group having 1 to 50 carbon atoms, an aryl group having 6 to 50 ring carbon atoms, or a monovalent heterocyclic group having 5 to 50 ring atoms. When two or more of each of R₄₁ to R₄₃, R₄₆, and R₄₉ to R₅₃ are present, the two or more of each of R₄₁ to R₄₃, R₄₆, and R₄₉ to R₅₃ may be the same as or different from each other.

In one embodiment, a substituent in the case of “substituted or unsubstituted” in the formula (2) is selected from the group consisting of
an alkyl group having 1 to 50 carbon atoms,
an aryl group having 6 to 50 ring carbon atoms, and
a monovalent heterocyclic group having 5 to 50 ring atoms.

In one embodiment, a substituent in the case of “substituted or unsubstituted” in the above formula (2) is selected from the group consisting of
an alkyl group having 1 to 18 carbon atoms,
an aryl group having 6 to 18 ring carbon atoms, and
a monovalent heterocyclic group having 5 to 18 ring atoms.

The compound according to an aspect of the present invention can be synthesized by using a known reaction or raw material suited for an intended product.

Specific examples of the compound represented by the formula (2) include those satisfying the formula (2) among the specific examples of the compound represented by the formula (1) described above.

### [Third Organic EL Device]

The third organic EL device according to an aspect of the present invention includes a cathode, an anode, and one or two or more organic layers disposed between the cathode and the anode, wherein at least one layer of the one or two or more organic layers includes the compound represented by the formula (2).

As for the other configurations of the third organic EL device, the content described for “the first organic EL device according to an aspect of the present invention” can be applied except that at least one layer of the one or two or more organic layers includes the compound represented by the formula (2) instead of the compound represented by the formula (1).

### EXAMPLES

### <Compound>

First components used for fabricating organic EL devices of Examples are shown below.

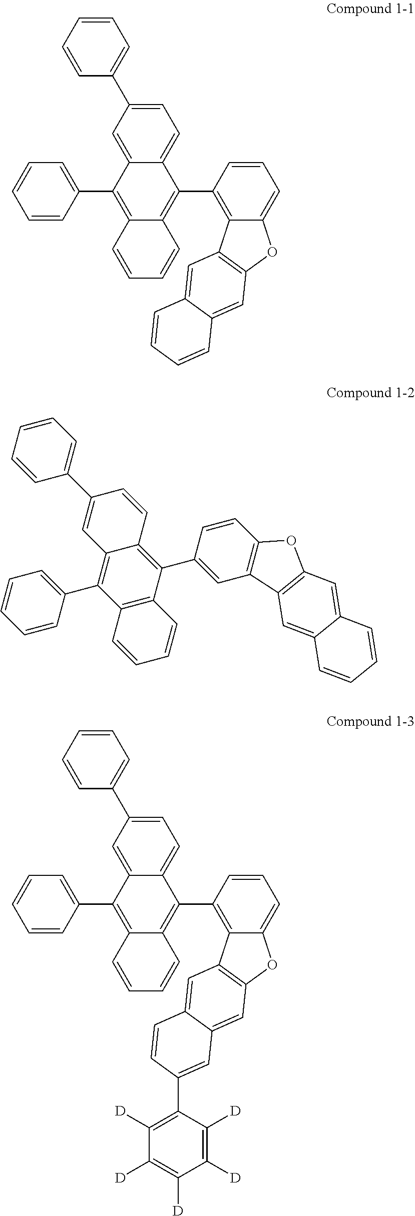 [SMILES: c1ccc(-c2ccc3c(-c4cccc5oc6cc7ccccc7cc6c45)c4ccccc4c(-c4ccccc4)c3c2)cc1 ; c1ccc(-c2ccc3c(-c4ccc5oc6cc7ccccc7cc6c5c4)c4ccccc4c(-c4ccccc4)c3c2)cc1 ; [2H]c1c([2H])c([2H])c(-c2ccc3cc4c(cc3c2)oc2cccc(-c3c5ccccc5c(-c5ccccc5)c5cc(-c6ccccc6)ccc35)c24)c([2H])c1[2H]]

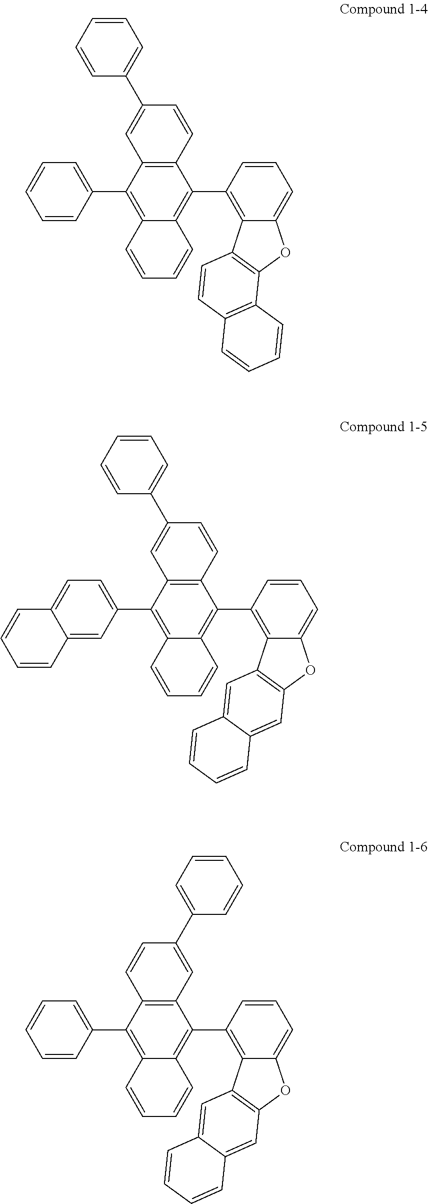 [SMILES: c1ccc(-c2ccc3c(-c4cccc5oc6c7ccccc7ccc6c45)c4ccccc4c(-c4ccccc4)c3c2)cc1 ; c1ccc(-c2ccc3c(-c4cccc5oc6cc7ccccc7cc6c45)c4ccccc4c(-c4ccc5ccccc5c4)c3c2)cc1 ; c1ccc(-c2ccc3c(-c4ccccc4)c4ccccc4c(-c4cccc5oc6cc7ccccc7cc6c45)c3c2)cc1]

Comparative compounds used for fabricating organic EL devices of Comparative Examples are shown below.

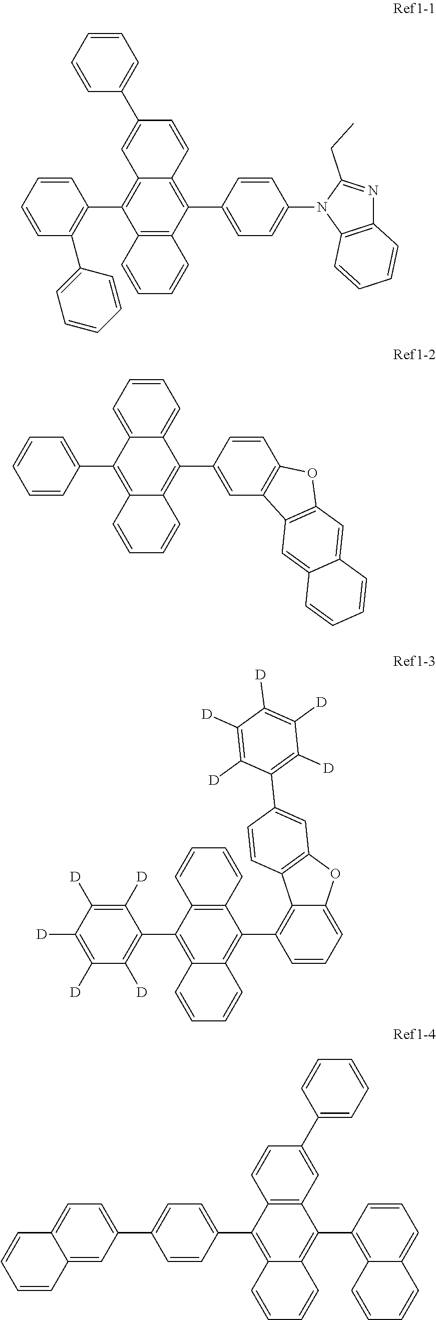

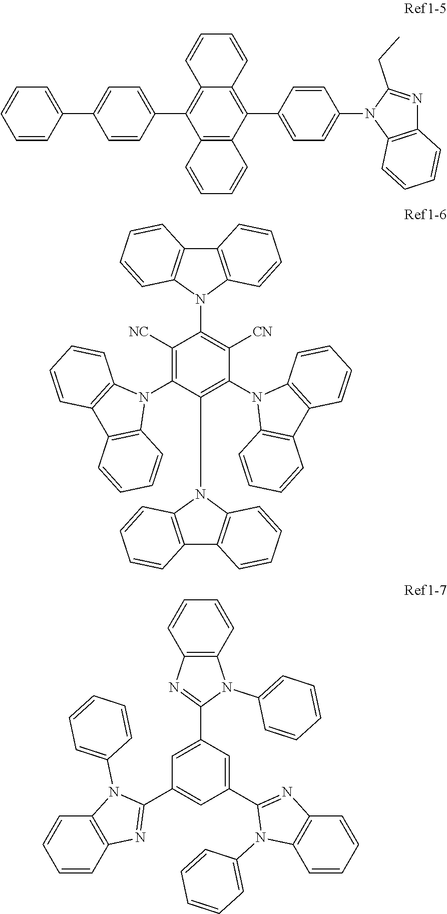

Second components used for fabricating the organic EL devices of Examples and Comparative Examples are shown below.

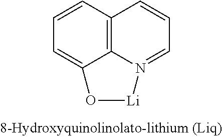

Structures of other compounds used for fabricating the organic EL devices of Examples and Comparative Examples are shown below.

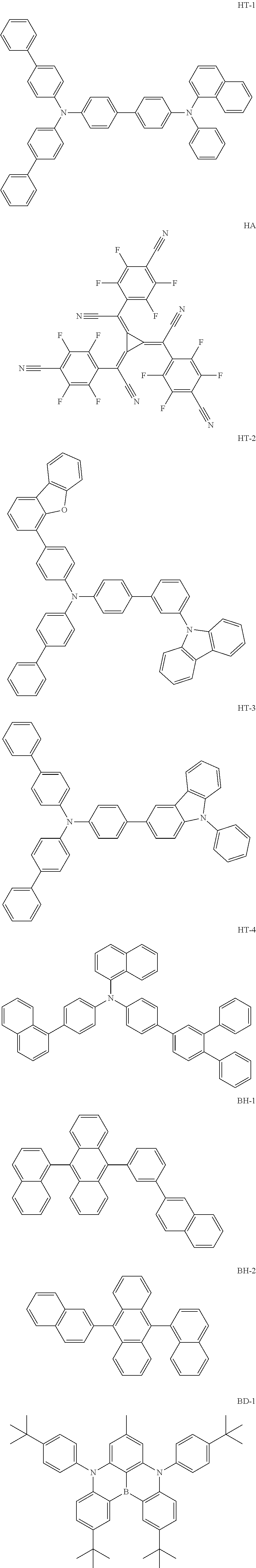

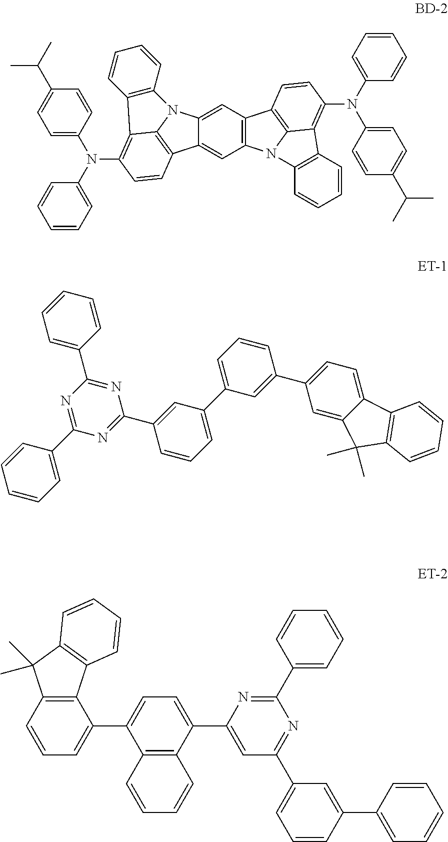

### Example 1

### <Fabrication of Organic EL Device>

An organic EL device was fabricated as follows.

A glass substrate of 25 mm×75 mm×1.1 mm thick with an ITO transparent electrode (anode) (manufactured by GEOMATEC Co., Ltd) was subjected to ultrasonic cleaning with isopropyl alcohol for 5 minutes, and then subjected to UV ozone cleaning for 30 minutes. The film thickness of ITO was 130 nm.

The cleaned glass substrate with a transparent electrode was mounted on a substrate holder in a vacuum deposition device, and first, compounds HT-1 and HA were co-deposited on the surface on the side on which the transparent electrode was formed so as to cover the transparent electrode so that the ratio of the compound HA was 3% by mass, thereby forming a first hole-transporting layer having a film thickness of 10 nm.

The compound HT-1 was deposited on the first hole-transporting layer to form a second hole-transporting layer having a film thickness of 80 nm.

A compound HT-2 was deposited on the second hole-transporting layer to form a third hole-transporting layer having a film thickness of 5 nm.

A compound BH-1 (host material) and a compound BD-1 (dopant material) were co-deposited on the third hole-transporting layer so that the ratio of the compound BD-1 was 1% by mass, thereby forming an emitting layer having a film thickness of 20 nm.

A compound ET-1 was deposited on the emitting layer to form a first electron-transporting layer having a film thickness of 5 nm.

A compound 1-1 and 8-hydroxyquinolinolato-lithium (Liq) were co-deposited on the first electron-transporting layer so that the ratio of Liq was 50% by mass, thereby forming a second electron-transporting layer having a film thickness of 25 nm.

Metal Yb was deposited on the second electron-transporting layer to form an electron-injecting layer having a film thickness of 1 nm.

Metal Al was deposited on the electron-injecting layer to form a cathode having a film thickness of 50 nm.

The device configuration of the organic EL device of Example 1 is schematically shown as follows.

ITO (130)/HT-1:HA (10:3%)/HT-1 (80)/HT-2 (5)/BH-1:BD-1 (20:1%)/ET-1 (5)/compound 1-1:Liq (25:50%)/Yb (1)/Al (50)

The number in parentheses represents the film thickness (unit: nm). In addition, the number expressed in percent in parentheses indicates the ratio (% by mass) of the latter compound in the layer.

### <Evaluation of Organic EL Device>

The fabricated organic EL device was evaluated as follows. The results are shown in Table 1.

### Driving Voltage

The initial characteristics of the organic EL device were measured at room temperature by driving at a direct current (DC) constant current of 10 mA/cm².

### EQE

A voltage was applied to the organic EL device so that the current density was 10 mA/cm² and the EL emission spectrum was measured with a spectroradiometer CS-2000 (manufactured by Konica Minolta, Inc). An external quantum efficiency (EQE) (%) was calculated from the obtained spectral-radiance spectrum. Hereinafter, this is referred to as “EQE at 10 mA/cm²”.

Next, EQE (%) was calculated in the same manner as described above, except that a voltage was applied to the organic EL device so that the current density was 0.1 mA/cm². Hereinafter, this is referred to as “EQE at 0.1 mA/cm²”.

An EQE ratio was determined based on the following relational expression.

$$EQE ratio = ( EQE at 0.1 mA / {cm}^{2} ) / ( EQE at 10 mA / {cm}^{2} )$$

### Examples 2 and 3

Organic EL devices were fabricated and evaluated in the same manner as in Example 1, except that compounds described in Table 1 were used instead of the compound 1-1 in formation of the second electron-transporting layer. The results are shown in Table 1.

### Comparative Examples 1 to 5

Organic EL devices were fabricated and evaluated in the same manner as in Example 1, except that compounds described in Table 1 were used instead of the compound 1-1 in formation of the second electron-transporting layer. The results are shown in Table 1.

TABLE 1

| | First component of | Volt- | EQE [%] | EQE [%] | |
|---|---|---|---|---|---|
| | second electron- | age | (10 mA/ | (0.1 mA/ | EQE |
| | transporting layer | [V] | cm²) | cm²) | ratio |
| Example 1 | Compound 1-1 | 3.60 | 10.3 | 10.3 | 1.00 |
| Example 2 | Compound 1-2 | 3.60 | 10.3 | 10.3 | 1.00 |
| Example 3 | Compound 1-3 | 3.60 | 8.3 | 7.8 | 0.94 |
| Comparative | Ref1-1 | 3.60 | 10.3 | 8.9 | 0.86 |
| Example 1 | | | | | |
| Comparative | Ref1-2 | 3.60 | 10.2 | 7.2 | 0.70 |
| Example 2 | | | | | |
| Comparative | Ref1-3 | 3.65 | 10.2 | 7.2 | 0.70 |
| Example 3 | | | | | |
| Comparative | Ref1-4 | 3.50 | 10.3 | 7.3 | 0.70 |
| Example 4 | | | | | |
| Comparative | Ref1-5 | 3.60 | 8.4 | 5.4 | 0.64 |
| Example 5 | | | | | |

Comparative Example 1 is the organic EL device using the compound (compound Ref1-1), which has the structure represented by the formula (M1) (electron-accepting structure) in the molecule thereof and has been conventionally used as an electron-transporting material or the like of an organic EL device, in which the EQE ratio was low, and EQE decreased at a low current density. The reason thereof is considered that the influence of the electron-accepting structure is larger in a low current density region as compared with a high current density region so that the carrier balance is lost due to excessive electrons.

In addition, the EQE ratio was also low in the organic EL devices of Comparative Examples 2 to 5 using the compounds not satisfying a specific structural condition.

The first organic EL device of the present invention (Examples 1 to 3) using the compound satisfying the specific structural condition exhibited a comparable driving voltage and comparable EQE at the high current density (10 mA/cm²) as compared with the organic EL devices of Comparative Examples 1 to 5. On the other hand, at the low current density (0.1 mA/cm²), the organic EL devices of Examples 1 and 2 exhibited EQE comparable to EQE at the high current density (10 mA/cm²), whereas the organic EL devices of Comparative Examples 1 to 5 exhibited low EQE.

### Example 4

### <Fabrication of Organic EL Device>

An organic EL device was fabricated as follows.

A glass substrate of 25 mm×75 mm×1.1 mm thick with an ITO transparent electrode (anode) (manufactured by GEOMATEC Co., Ltd) was subjected to ultrasonic cleaning with isopropyl alcohol for 5 minutes, and then subjected to UV ozone cleaning for 30 minutes. The film thickness of ITO was 130 nm.

The cleaned glass substrate with a transparent electrode was mounted on a substrate holder in a vacuum deposition device, and first, compounds HT-3 and HA were co-deposited on the surface on the side on which the transparent electrode was formed so as to cover the transparent electrode so that the ratio of the compound HA was 3% by mass, thereby forming a first hole-transporting layer having a film thickness of 10 nm.

The compound HT-3 was deposited on the first hole-transporting layer to form a second hole-transporting layer having a film thickness of 80 nm.

A compound HT-4 was deposited on the second hole-transporting layer to form a third hole-transporting layer having a film thickness of 5 nm.

A compound BH-2 (host material) and a compound BD-2 (dopant material) were co-deposited on the third hole-transporting layer so that the ratio of the compound BD-1 was 1% by mass, thereby forming an emitting layer having a film thickness of 20 nm.

A compound ET-2 was deposited on the emitting layer to form a first electron-transporting layer having a film thickness of 5 nm.

A compound 1-4 and 8-hydroxyquinolinolato-lithium (Liq) were co-deposited on the first electron-transporting layer so that the ratio of Liq was 50% by mass, thereby forming a second electron-transporting layer having a film thickness of 25 nm.

Metal Yb was deposited on the second electron-transporting layer to form an electron-injecting layer having a film thickness of 1 nm.

Metal Al was deposited on the electron-injecting layer to form a cathode having a film thickness of 50 nm.

The device configuration of the organic EL device of Example 4 is schematically shown as follows.

ITO (130)/HT-3:HA (10:3%)/HT-3 (80)/HT-4 (5)/BH-2:BD-2 (20:1%)/ET-2 (5)/compound 1-4:Liq (25:50%)/Yb (1)/Al (50)

The number in parentheses represents the film thickness (unit: nm). In addition, the number expressed in percent in parentheses indicates the ratio (% by mass) of the latter compound in the layer.

### Examples 5 and 6

Organic EL devices were fabricated and evaluated in the same manner as in Example 4, except that compounds described in Table 2 were used instead of the compound 1-4 in formation of the second electron-transporting layer. The results are shown in Table 2.

TABLE 2

| | First component of | Volt- | EQE [%] | EQE [%] | |
|---|---|---|---|---|---|
| | second electron- | age | (10 mA/ | (0.1 mA/ | EQE |
| | transporting layer | [V] | cm²) | cm²) | ratio |
| Example 4 | Compound 1-4 | 3.61 | 10.5 | 9.5 | 0.90 |
| Example 5 | Compound 1-5 | 3.61 | 10.7 | 9.7 | 0.91 |
| Example 6 | Compound 1-6 | 3.58 | 10.6 | 9.8 | 0.92 |

### <Evaluation of Physical Properties of Compounds>

For the first components and the comparative compounds, a giant surface polarization (GSP) slope (GSP_slope) and the lowest unoccupied molecular orbital energy level (LUMO) were measured by the following measurement method. The results are shown in Table 3.

### Giant Surface Polarization (GSP) Slope

The giant surface polarization (GSP) slope (GSP_slope) was obtained by measuring the film thickness dependence of a surface potential.

An object to be measured was deposited by 20 nm on an ITO substrate at a deposition rate of 2 Å/s under a vacuum degree of 10⁻⁵ Pa while shielding the object to be measured in a chamber from light, and the surface potential (unit: mV) of the deposited film was measured. This was repeated 5 times, and a value of a slope in a diagram of plots having the surface potential (unit: mV) on the vertical axis and the film thickness (unit: nm) on the horizontal axis was obtained by fitting the plots to a straight line by a least squares method, and was defined as GSP_slope (unit: mV/nm). For the measurement of the surface potential in vacuum, a Kelvin probe device (manufactured by Tokyo Instruments Inc., “UHV Kelvin probe”) was used.

Both the deposition and the surface potential measurement were performed under light shielding and vacuum. In addition, a sample was placed in the same chamber (under light shielding and vacuum) while repeating the deposition and the measurement. The phrase “this was repeated 5 times” means that deposition by 20 nm was performed on the sample (ITO substrate) and the surface potential was measured, deposition by 20 nm was additionally performed on the same sample (so that the thickness of the deposited film was 40 nm in total) and the surface potential was measured, deposition by 20 nm was additionally performed on the same sample (so that the thickness of the deposited film was 60 nm in total) and the surface potential was measured, deposition by 20 nm was additionally performed on the same sample (so that the thickness of the deposited film was 80 nm in total) and the surface potential was measured, and then deposition by 20 nm was additionally performed on the same sample (so that the thickness of the deposited film was 100 nm in total) and the surface potential was measured.

(Reference Document) Y. Noguchi, Y. Miyazaki, Y. Tanaka, N. Sato, Y. Nakayama, T. D. Schmidt, W. Brutting, H. Ishii, Charge accumulation at organic semiconductor interfaces due to a permanent dipole moment and its orientational order in bilayer devices. J. Appl. Phys. 111, 114508 (2012).

### Lowest Unoccupied Molecular Orbital Energy Level (LUMO)

The lowest unoccupied molecular orbital energy level (LUMO: lowest unoccupied molecular orbital) was calculated by the following mathematical expression (Math. 1Y) using a differential pulse voltammetry method (unit: eV).

$$LUMO = - 1.19 \times ( Ere - Efc ) - 4.78 eV \qquad ( Math . 1 Y )$$

In the mathematical expression (Math. 1Y), Ere and Efc are as follows.
Ere: First reduction potential of object to be measured (DPV, Negative scan)
Efc: First oxidation potential of ferrocene (DPV, Positive scan), (ca. +0.55 VvsAg/AgCl)

The oxidation-reduction potential was measured by a differential pulse voltammetry (DPV) method using an electrochemical analyzer (manufactured by ALS: CH1852D). In a sample solution used for the measurement, N,N-dimethylformamide (DMF) was used as a solvent, and the object to be measured was dissolved so as to have a concentration of 1.0 mmol/L. A supporting electrolyte was prepared by dissolving tetrabutylammonium hexafluorophosphate (TBHP) so as to have a concentration of 100 mmol/L.

A glassy carbon electrode was used as a working electrode. A platinum (Pt) electrode was used as a counter electrode.
(Reference Document) M. E. Thompson, et. al., Organic Electronics, 6 (2005), p. 11-20, Organic Electronics, 10 (2009), p. 515-520

### <Structural Condition for Compounds>

Table 3 shows whether or not the structural condition that “does not have the structures represented by the formulas (M1) to (M4) in the molecule thereof” is satisfied for the first components and the comparative compounds.

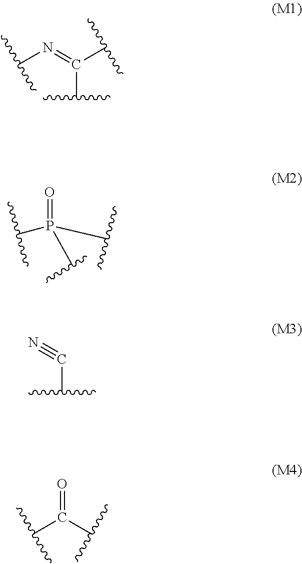

### Example 7

An organic EL device was fabricated and evaluated in the same manner as in Example 1. The results are shown in Table 3.

### Example 8

An organic EL device was fabricated and evaluated in the same manner as in Example 7, except that the compound 1-2 was used instead of the compound 1-1 in formation of the second electron-transporting layer. The results are shown in Table 3.

### Comparative Examples 6 to 10

Organic EL devices were fabricated and evaluated in the same manner as in Example 7, except that compounds described in Table 3 were used instead of the compound 1-1 in formation of the second electron-transporting layer. The results are shown in Table 3.

TABLE 3

| | First component of | GSP_slope of | LUMO of first | | | |
|---|---|---|---|---|---|---|
| | second electron- | first component | component | Structural | Voltage | |
| | transporting layer | [mV/nm] | [eV] | condition | [V] | EQE ratio |
| Example 7 | Compound 1-1 | 51 | −2.19 | Satisfaction | 3.60 | 1.00 |
| Example 8 | Compound 1-2 | 42 | −2.13 | Satisfaction | 3.60 | 1.00 |
| Comparative | Ref1-1 | 47 | −2.26 | Non-satisfaction | 3.60 | 0.86 |
| Example 6 | | | | | | |
| Comparative | Ref1-2 | 37 | −2.07 | Satisfaction | 3.60 | 0.70 |
| Example 7 | | | | | | |
| Comparative | Ref1-3 | 35 | −2.09 | Satisfaction | 3.65 | 0.70 |
| Example 8 | | | | | | |
| Comparative | Ref1-5 | 51 | −2.80 | Non-satisfaction | 6.30 | 0.45 |
| Example 9 | | | | | | |
| Comparative | Ref1-6 | 43 | −1.86 | Non-satisfaction | 4.80 | 0.88 |
| Example 10 | | | | | | |

As seen from Table 3, it was found that, in the second organic EL device of the present invention (Examples 3 and 4) using the compound having GSP_slope and LUMO within predetermined ranges and satisfying the specific structural condition, the driving voltage is low, the EQE ratio was high, and EQE was maintained even at the low current density (0.1 mA/cm²).

In Ref1-1, GSP_slope and LUMO were within the predetermined ranges, but the above-described structural condition was not satisfied (the structure represented by the formula (M1) was included in the molecule thereof). It was found that the EQE ratio was low, and EQE decreased at the low current density in Comparative Example 6 using Ref1-1.

In Ref1-2 and Ref1-3, LUMO was within the predetermined range, and the above-described structural condition was satisfied, but GSP_slope was outside the predetermined range. It was found that, in Comparative Examples 7 and 8 using these compounds, the voltage was comparable to that in Examples 7 and 8 since LUMO was within the predetermined range, but the EQE ratio was low, and EQE decreased at the low current density.

In Ref1-5, GSP_slope was within the predetermined range, but LUMO was outside the predetermined range, and the above-described structural condition was not satisfied (the structure represented by the formula (M3) was included in the molecule thereof). In Ref1-6, GSP_slope was within the predetermined range, but LUMO was outside the predetermined range, and the above-described structural condition was not satisfied (the structure represented by the formula (M1) was included in the molecule thereof). It was found that, in Comparative Examples 9 and 10 using these compounds, the driving voltage was extremely high since LUMO was outside the predetermined range, the EQE ratio was low, and EQE decreased at the low current density.

### <Synthesis of Compound>

### (Synthesis Example 1) Synthesis of Compound 1-3

Compound 1-3 was synthesized by the following synthesis route.

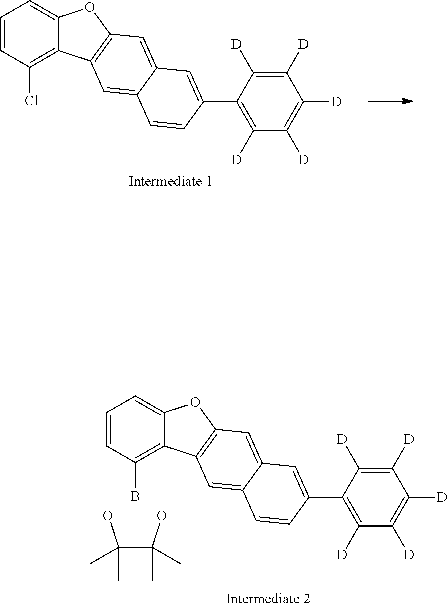

### (1) Synthesis of Intermediate 2

NMP (360 mL) was added to Intermediate 1 (36.3 g), bis(pinacolato)diboron (55.2 g) and potassium acetate (32.0 g), and the mixture was heated to 105° C. and bubbled with argon for 30 minutes. PCy₃ (2.4 g) and Pd₂(dba)₃ (4.0 g) were added, and the mixture was heated at 105° C. for 2.5 hours with stirring under an argon atmosphere. The reaction solution was allowed to cool to room temperature, filtered through celite, and washed with ethyl acetate. The filtrate was washed with water three times, and the residue obtained by concentrating the organic phase was subjected to column chromatography to obtain Intermediate 2 (34.7 g, yield: 75%) as a white solid.

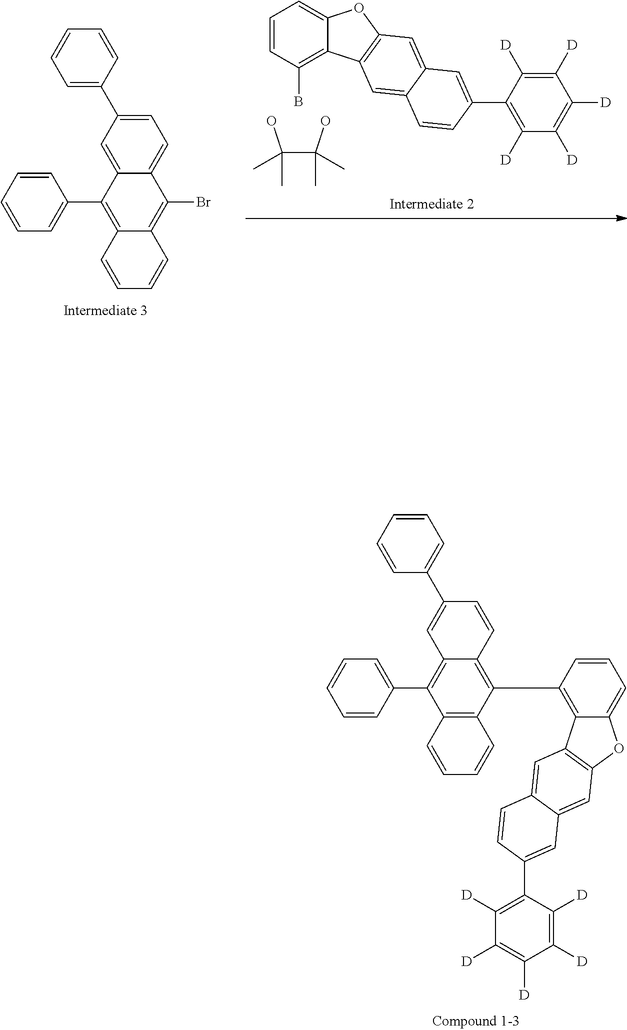

### (2) Synthesis of Compound 1-3

Toluene (180 mL) and a 2 M aqueous potassium carbonate solution (22 mL) were added to Intermediate 3 (9.2 g), Pd(PPh₃)₄ (0.5 g) and Aliquat336 (0.9 g), and the mixture was bubbled with argon for 30 minutes. The resultant was heated to 85° C. and added with Intermediate 2 (8.9 g), and then the mixture was heated and stirred for 19 hours. The reaction solution was allowed to cool to room temperature, and then a crude product, obtained by collecting solid precipitated by adding water, was purified by silica gel chromatography, and recrystallized with toluene to obtain Compound 1-3 as a white solid (6.3 g, yield: 46%).

The result of mass spectrometric analysis was m/e=628 to the molecular weight 627.80, and Compound 1-3 was identified as an intended product.

Although several embodiments and/or examples of the present invention have been described above in detail, for those skilled in the art, it is easy to add numerous modifications to these exemplary embodiments and/or examples without substantially departing from the novel teachings and effects of the present invention. Therefore, many of these modifications fall within the scope of the present invention.

The documents described in the specification and the specification of Japanese application(s) on the basis of which the present application claims Paris convention priority are incorporated herein by reference in its entirety.

1. An organic electroluminescence device comprising
a cathode,
an anode, and
one or two or more organic layers disposed between the cathode and the anode,
wherein at least one layer of the one or two or more organic layers comprises a first component and a second component,
the first component is a compound represented by the following formula (1), and
the second component is selected from the group consisting of an alkali metal, an alkali metal compound, an alkaline earth metal, an alkaline earth metal compound, a rare earth metal, a rare earth metal compound, an organic metal complex containing an alkali metal, an organic metal complex containing an alkaline earth metal, and an organic metal complex containing a rare earth metal: 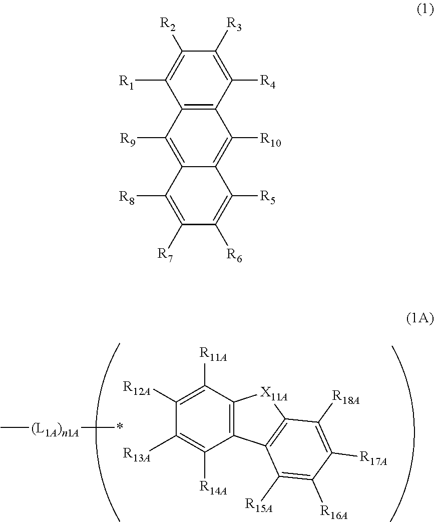
wherein in the formula (1),
at least one of R₁ to R₁₀ is a group represented by the formula (1A);
R₁ to R₁₀ that are not the group represented by the formula (1A) are independently a hydrogen atom, or a substituent A; provided that at least three of R₁ to R₁₀ are independently the group represented by the formula (1A), the substituent A, or a hydrogen atom which is a deuterium atom;
in the formula (1A),
L_{1A} is
a single bond,
a substituted or unsubstituted arylene group having 6 to 50 ring carbon atoms, or
a substituted or unsubstituted divalent heterocyclic group having 5 to 50 ring atoms;
n1A is an integer of 0 to 3;
when n1A is 0, (L_{1A})_{n1A} is a single bond;
when n1A is 2 or 3, the plurality of L_{1A}'s is connected in series with each other, and a structure in parentheses is bonded to L_{1A} farthest from the anthracene skeleton; the plurality of L_{1A}'s may be the same as or different from each other;
X_{11A} is C(R_{21A})(R_{22A}), N(R_{23A}), O, or S;
one or more sets of the adjacent two or more of R_{11A} to R_{18A} form a substituted or unsubstituted single ring by bonding with each other, form a substituted or unsubstituted fused ring by bonding with each other, or do not form any of the rings;
when the substituted or unsubstituted single ring is formed, one of atoms forming the single ring is bonded to L_{1A}, or one of R_{11A} to R_{18A} that do not contribute to the formation of the single ring, and R_{21A} to R_{23A} represents a bond to L_{1A};
when the substituted or unsubstituted fused ring is formed, one of atoms forming the fused ring is bonded to L_{1A}, or one of R_{11A} to R_{18A} that do not contribute to the formation of the fused ring, and R_{21A} to R_{23A} represents a bond to L_{1A};
when the single ring and the fused ring are not formed, one of R_{11A} to R_{18A} and R_{21A} to R_{23A} represents a bond to L_{1A};
R_{11A} to R_{18A} and R_{21A} to R_{23A} that do not represent the bond to L_{1A} and that do not form any of the rings are independently a hydrogen atom or a substituent A;
when two or more groups represented by the formula (1A) are present, the two or more groups represented by the formula (1A) may be the same as or different from each other;
the substituent A is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms, 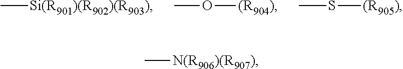
a halogen atom, a nitro group,
a substituted or unsubstituted aryl group having 6 to 50 ring carbon atoms, or
a substituted or unsubstituted monovalent heterocyclic group having 5 to 50 ring atoms;
R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more substituents A are present, the two or more substituents A may be the same as or different from each other; and
when two or more of each of R₉₀₁ to R₉₀₇ are present, the two or more of each of R₉₀₁ to R₉₀₇ may be the same as or different from each other,
provided that the compound represented by the formula (1) does not have a structure represented by the following formula (M1), a structure represented by the following formula (M2), a structure represented by the following formula (M3), and a structure represented by the following formula (M4) in the molecule thereof:

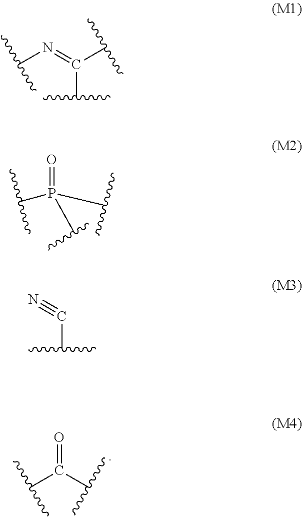

2. The organic electroluminescence device according to claim 1, wherein at least three of R₁ to R₁₀ are independently the group represented by the formula (1A), or the substituent A.

3. The organic electroluminescence device according to claim 1, wherein two of R₁ to R₁₀ are independently the substituent A, and one of the other R₁ to R₁₀ is the group represented by the formula (1A).

4. The organic electroluminescence device according to claim 1, wherein R₉ and R₁₀ are independently the group represented by the formula (1A), or the substituent A.

5. The organic electroluminescence device according to claim 1, wherein one of R₉ and R₁₀ is the group represented by the formula (1A), and the other of R₉ and R₁₀ is the substituent A.

6. The organic electroluminescence device according to claim 1, wherein R₂, R₉, and R₁₀ are independently the group represented by the formula (1A), or the substituent A.

7. The organic electroluminescence device according to claim 6, wherein one of R₉ and R₁₀ is the group represented by the formula (1A), and the other of R₉ and R₁₀ and R₂ are independently the substituent A.

8. The organic electroluminescence device according to claim 1, wherein the group represented by the formula (1A) is a group represented by any one of the following formulas (1A-1) to (1A-3): 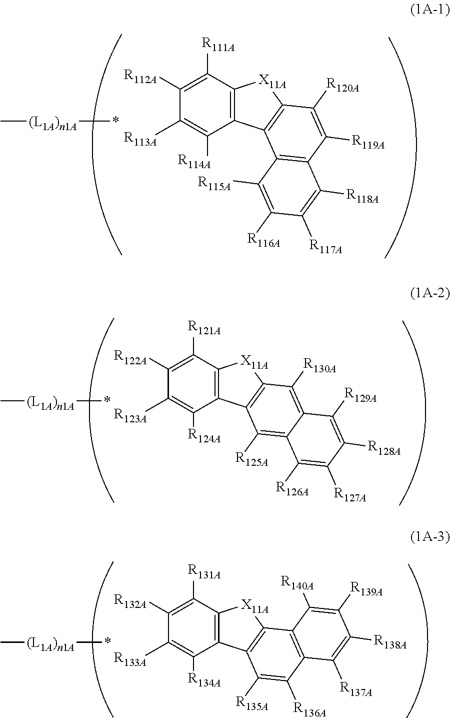
wherein in the formulas (1A-1) to (1A-3), L_{1A} and n1A are as defined in the formula (1);
X_{11A} is C(R_{21A})(R_{22A}), N(R_{23A}), O, or S;
one of R_{21A} to R_{23A}, R_{111A} to R_{120A}, R_{121A} to R_{130A}, and R_{131A} to R_{140A} represents a bond to L_{1A};
R_{21A} to R_{23A}, R_{111A} to R_{120A}, R_{121A} to R_{130A}, and R_{131A} to R_{140A} that do not represent the bond to L_{1A} are independently a hydrogen atom, or a substituent A; and
the substituent A is as defined in the formula (1).

9. The organic electroluminescence device according to claim 1, wherein X_{11A} is O or S.

10. The organic electroluminescence device according to claim 1, wherein the compound represented by the formula (1) is a compound represented by any one of the following formulas (1-1) to (1-3): 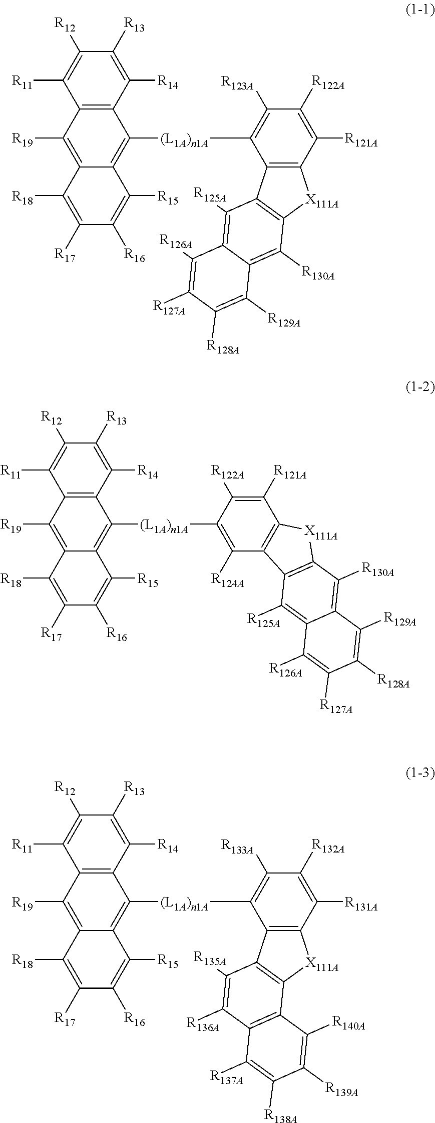
wherein in the formulas (1-1) to (1-3), L_{1A} and n1A are as defined in the formula (1);
X_{111A} is O or S;
R₁₂ and R₁₉ are independently a substituent A;
R₁₁, R₁₃ to R₁₈, and R_{121A} to R_{140A} are independently a hydrogen atom or a substituent A; and
the substituent A is as defined in the formula (1),
provided that the compound represented by any one of the formulas (1-1) to (1-3) does not have a structure represented by the following formula (M1), a structure represented by the following formula (M2), a structure represented by the following formula (M3), and a structure represented by the following formula (M4) in the molecule thereof:

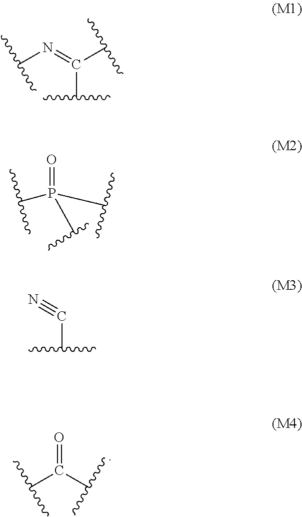

11. The organic electroluminescence device according to claim 1, wherein L_{1A} is a single bond.

12. The organic electroluminescence device according to claim 1, wherein the compound represented by the formula (1) is a compound represented by any one of the following formulas (1-11) to (1-41): 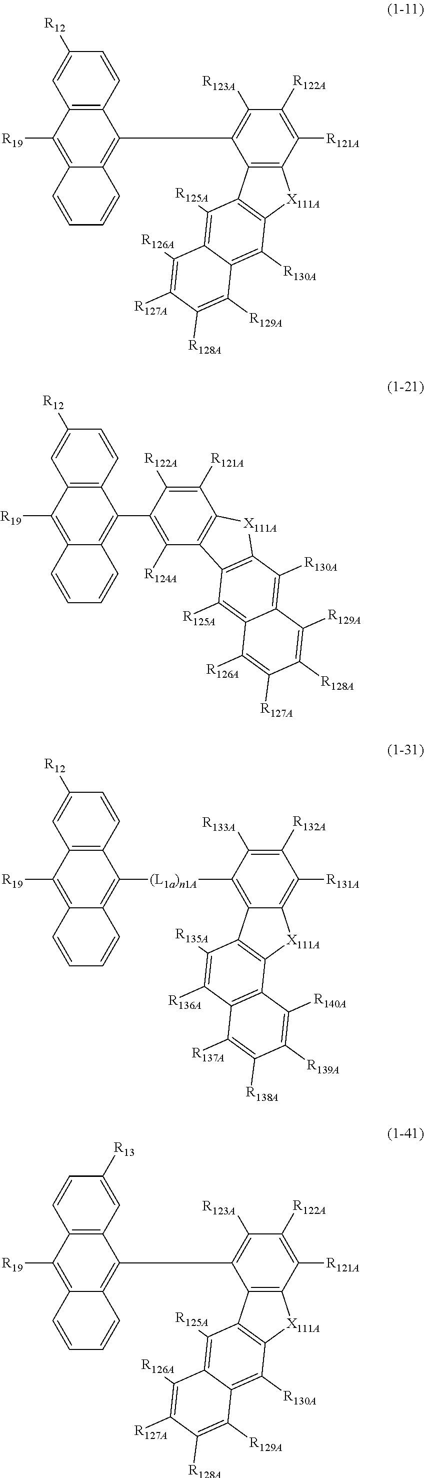
wherein in the formulas (1-11) to (1-41),
X_{111A} is 0 or S;
R₁₂, R₁₃, and R₁₉ are independently a substituent A;
R_{121A} to R_{140A} are independently a hydrogen atom or a substituent A; and
the substituent A is as defined in the formula (1),
provided that the compound represented by any one of the formulas (1-11) to (1-41) does not have a structure represented by the following formula (M1), a structure represented by the following formula (M2), a structure represented by the following formula (M3), and a structure represented by the following formula (M4) in the molecule thereof:

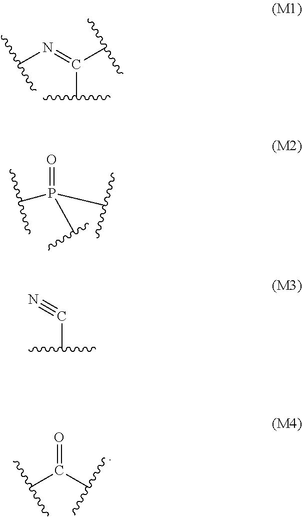

13. An organic electroluminescence device comprising
a cathode,
an anode, and
one or two or more organic layers disposed between the cathode and the anode,
wherein at least one layer of the one or two or more organic layers includes a first component and a second component,
the first component is a compound that satisfies the following formulas (R₁) and (R₂) and does not have structures represented by the following formulas (M1) to (M4) in the molecule thereof, and
the second component is selected from the group consisting of an alkali metal, an alkali metal compound, an alkaline earth metal, an alkaline earth metal compound, a rare earth metal, a rare earth metal compound, an organic metal complex containing an alkali metal, an organic metal complex containing an alkaline earth metal, and an organic metal complex containing a rare earth metal:
$$40 mV / nm \leq GSP\_slope \qquad ( R1 )$$
$$- 2.8 eV < LUMO < - 1.86 eV \qquad ( R2 )$$ 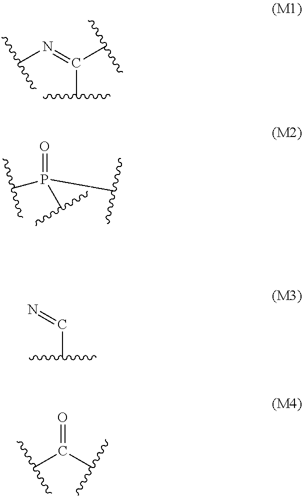
wherein in the formula (R1), GSP_slope represents a giant surface polarization slope; and
in the formula (R2), LUMO represents a lowest unoccupied molecular orbital energy level.

14. The organic electroluminescence device according to claim 13, wherein the first component satisfies the following formula (R1-1):
$$GSP\_slope \leq 60 mV / nm \qquad ( R1 - 1 )$$
wherein in the formula (R1-1), GSP_slope is as defined in the formula (R1).

15. The organic electroluminescence device according to claim 13, wherein the first component satisfies the following formula (R1-2):
$$41 mV / nm \leq GSP\_slope \qquad ( R1 - 2 )$$
wherein in the formula (R1-2), GSP_slope is as defined in the formula (R1).

16. The organic electroluminescence device according to claim 13, wherein the first component satisfies the following formula (R1-3):
$$41 mV / nm \leq GSP\_slope \leq 60 mV / nm \qquad ( R1 - 3 )$$
wherein in the formula (R1-3), GSP_slope is as defined in the formula (R1).

17. The organic electroluminescence device according to claim 13, wherein the first component satisfies the following formula (R1-4):
$$42 mV / nm \leq GSP\_slope \leq 51 mV / nm \qquad ( R1 - 4 )$$
wherein in the formula (R1-4), GSP_slope is as defined in the formula (R1).

18. The organic electroluminescence device according to claim 13, wherein the first component satisfies the following formula (R₂-1):
$$- 2.6 eV \leq LUMO \leq - 2. eV \qquad ( R2 - 1 )$$
wherein in the formula (R2-1), LUMO is as defined in the formula (R2).

19. The organic electroluminescence device according to claim 13, wherein the first component satisfies the following formula (R₂-2):
$$- 2.26 eV \leq LUMO \leq - 2.09 eV \qquad ( R2 - 2 )$$
wherein in the formula (R2-2), LUMO is as defined in the formula (R2).

20. The organic electroluminescence device according to claim 13, wherein the first component satisfies the following formula (R1-3) and the following formula (R2-2):
$$41 mV / nm \leq GSP\_slope \leq 60 mV / nm \qquad ( R1 - 3 )$$
$$- 2.26 eV \leq LUMO \leq - 2.09 eV \qquad ( R2 - 2 )$$
wherein in the formula (R1-3), GSP_slope is as defined in the formula (R1); and in the formula (R2-2), LUMO is as defined in the formula (R2).

21. The organic electroluminescence device according to claim 1, wherein the second component is selected from the group consisting of an alkali metal, an alkali metal compound, and an organic metal complex containing an alkali metal.

22. The organic electroluminescence device according to claim 1, wherein the amount of the first component is 30 to 70% by mass based on the total amount of the first component and the second component.

23. The organic electroluminescence device according to claim 1, which comprises the anode, an emitting layer, an electron-transporting zone, and the cathode in this order, wherein at least one layer in the electron-transporting zone comprises the first component and the second component.

24. The organic electroluminescence device according to claim 23, wherein the electron-transporting zone comprises at least a first layer and a second layer in this order from the emitting layer side, and
the second layer comprises the first component and the second component.

25. The organic electroluminescence device according to claim 24, wherein the second layer substantially consists of the first component and the second component.

26. An electronic apparatus comprising the organic electroluminescence device according to claim 1.

27. A compound represented by the following formula (2): 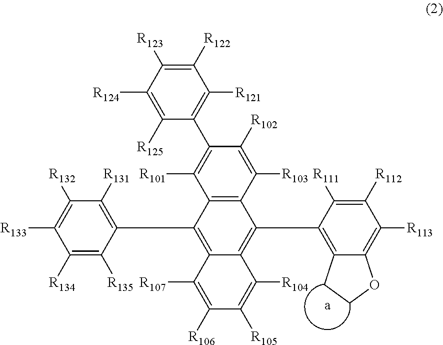
wherein in the formula (2),
a ring a is
a substituted or unsubstituted aromatic hydrocarbon ring having 10 to 50 ring carbon atoms, or
a substituted or unsubstituted heterocyclic ring having 6 to 50 ring atoms;
R₁₀₁ to R₁₀₇, R₁₁₁ to R₁₁₃, R₁₂₁ to R₁₂₅, and R₁₃₁ to R₁₃₅ are independently a hydrogen atom, or a substituent R,
provided that at least one of R₁ₗ to R₁₁₃ is the substituent R, or the ring a has at least one substituent;
the substituent R is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms, 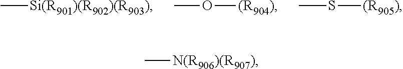
a halogen atom, a cyano group, a nitro group,
a substituted or unsubstituted aryl group having 6 to 50 ring carbon atoms, or
a substituted or unsubstituted monovalent heterocyclic group having 5 to 50 ring atoms;
R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more of each of R₉₀₁ to R₉₀₇ are present, the two or more of each of R₉₀₁ to R₉₀₇ may be the same as or different from each other; and
when two or more substituents R are present, the two or more substituents R may be the same as or different from each other.

28. The compound according to claim 27, wherein the compound represented by the formula (2) is a compound represented by the following formula (2-1): 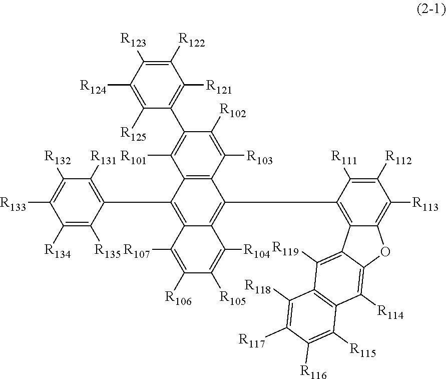
wherein in the formula (2-1),
R₁₀₁ to R₁₀₇, R₁₁₁ to R₁₁₉, R₁₂₁ to R₁₂₅, and R₁₃₁ to R₁₃₅ are independently a hydrogen atom, or a substituent R; provided that at least one of R₁ₗ to R₁₁₉ is the substituent R; and
the substituent R is as defined in the formula (2).

29. The compound according to claim 27, wherein the compound represented by the formula (2) is a compound represented by the following formula (2-11): 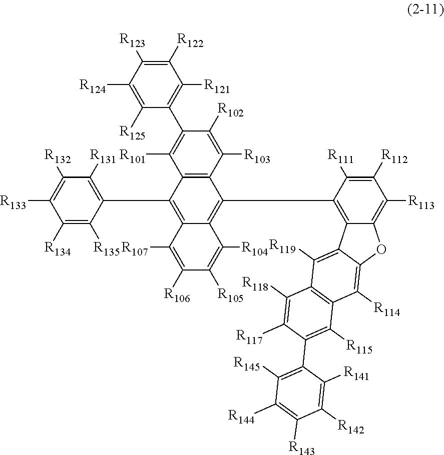
wherein in the formula (2-11),
R₁₀₁ to R₁₀₇, R₁₁₁ to R₁₁₅, R₁₁₇ to R₁₁₉, R₁₂₁ to R₁₂₅, R₁₃₁ to R₁₃₅, and R₁₄₁ to R₁₄₅ are independently a hydrogen atom, or a substituent R; and
the substituent R is as defined in the formula (2).
